(19) United States
(12) Patent Application Publication
(10) Pub. No.: US 2025/0057933 A1
(43) Pub. Date: Feb. 20, 2025

(54) Rhinovirus Vaccine

(71) Applicant: IP2IPO INNOVATIONS LIMITED, London (GB)

(72) Inventors: Sebastian Johnston, London (GB); Stephen Shaw, Cambridge (GB); Paul Hamblin, Stevenage (GB); Aurelie Bornot, Stevenage (GB); Piero Ricchiuto, Boston, MA (US); Claus Bendtsen, Molndal (SE)

(21) Appl. No.: 18/817,094

(22) Filed: Aug. 27, 2024

Related U.S. Application Data
(63) Continuation of application No. PCT/GB2023/050427, filed on Feb. 27, 2023.

(30) Foreign Application Priority Data
Feb. 28, 2022 (GB) 2202738.7

(51) Int. Cl. A61K 39/125; A61K 39/00; A61P 31/16; C12N 7/00

(52) U.S. Cl. CPC A61K 39/125; A61P 31/16; C12N 7/00; A61K 2039/53; A61K 2039/70; C12N 2770/32734

(57) ABSTRACT

The invention relates to immunogenic compositions, and in particular, to immunogenic compositions for preventing, treating or ameliorating human rhinovirus (RV) infections. The invention is especially concerned with RV VP0 peptides (or proteins) and polynucleotides encoding such peptides, and their use in immunogenic compositions for eliciting an immune response and preventing rhinovirus infections.

### CROSS REFERENCE OF RELATED APPLICATION

This application claims the benefit of Great Britain Application No. 2202738.7 filed Feb. 28, 2022 and International Patent Application No. PCT/GB2023/050427 filed on Feb. 27, 2023, both of which are incorporated by reference in their entirety herein

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: 70724_SeqListing.xml; Size: 44,926 bytes; Created: Aug. 27, 2024.

### DETAILED DESCRIPTION

The present invention relates to immunogenic compositions, and in particular, to immunogenic compositions for preventing, treating or ameliorating human rhinovirus (RV) infections. The invention is especially concerned with RV VP0 peptides (or proteins) and polynucleotides encoding such peptides, and their use in immunogenic compositions for eliciting an immune response and preventing rhinovirus infections.

Rhinoviruses (RVs) are picornaviruses, which are small single stranded RNA viruses with a genome size of approximately 6.8-7.2 Kb [1]. RVs are the major cause of common colds, of virus-induced wheezing illnesses in early childhood and of acute attacks (exacerbations) of lung diseases such as asthma, bronchiectasis, chronic obstructive pulmonary disease (COPD), cystic fibrosis and chronic fibrosing lung diseases [2,3]. RVs cause around 75% to 90% of such illnesses.

There are currently no effective anti-RV treatments and no anti-RV vaccines. A major obstacle to the development of a RV vaccine has been the large number of genetically and antigenically distinct circulating strains. A recent study estimated that approximately 180 genetically distinct strains of RV are in circulation [4]. These strains are classified based on their genome identity into three species: RV-A, RV-B and RV-C [1,5]. RV-A and RV-C are the largest species numerically and are most important in terms of clinical illness, with RV-A and RV-Cs responsible for most exacerbations of asthma and COPD and childhood wheezing illnesses resulting in hospitalisation. However, RV-Bs are also associated with an increased risk of wheezing illnesses [7].

There is, therefore, a need to provide an immunogenic composition that is capable of protecting individuals against many, if not all, RV strains.

The inventors, using in depth bioinformatics analysis, have identified RV VP0 proteins from single representative strains that, when used as vaccine immunogens, surprisingly evoke cellular immunity against all the other members of that RV species.

Therefore, according to a first aspect of the invention, there is provided an immunogenic composition comprising at least one isolated human rhinovirus (RV) VP0 peptide, or an isolated polynucleotide encoding the peptide, wherein the peptide is selected from a group consisting of: an RV-C VP0 peptide; an RV-B VP0 peptide; RV-A28 VP0; and RV-A89 VP0, or a variant or fragment thereof.

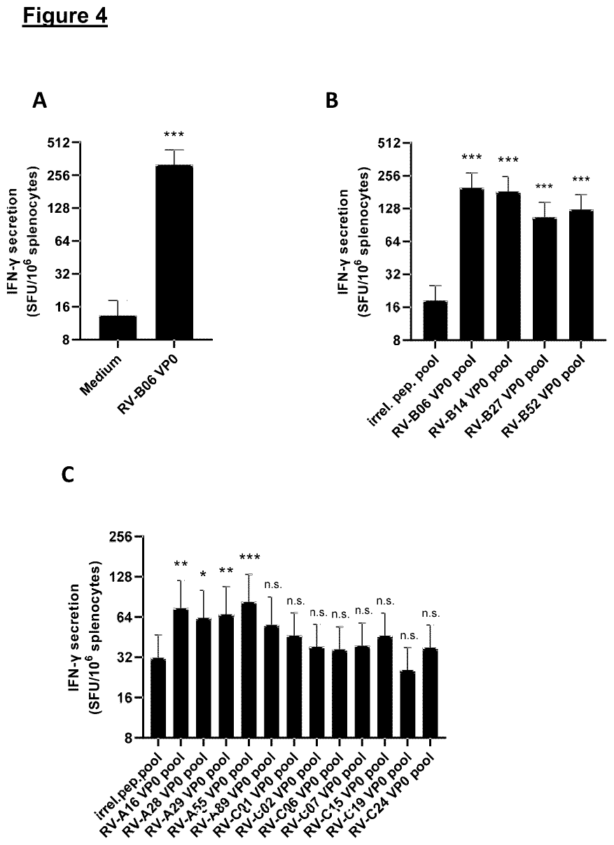

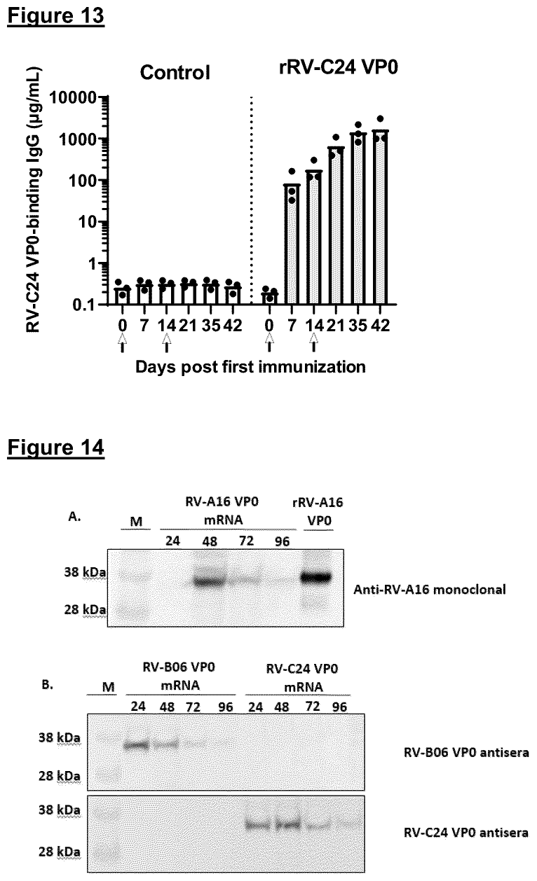

As shown in the examples, the inventors surprisingly discovered that the novel rhinovirus immunogens are able to elicit broad cellular immune responses that cross-react with other RV strains from the same species. For example, as shown in FIGS. 4, 8, and 9 splenocytes from RV-B06 VP0 immunised mice cross react with VP0 peptides from other RV-B species members, and splenocytes from either RV-C19 VP0 or RV-C24 VP0 immunised mice cross react with VP0 peptides from other RV-C species members, respectively. Additionally, the inventors have demonstrated that immunisation with mRNA encoding the RV VP0 peptides, elicits a broad cellular immune response. For example, as shown in FIG. 15, the inventors demonstrated that immunisation of mice with RV-C24 VP0 mRNA evokes cellular immunity that is cross-reactive with other members of C species of RV. Advantageously, therefore, the immunogenic composition according to the invention overcomes the issue of antigenic heterogeneity across RV strains, which has significantly hampered RV vaccine development to date.

In a preferred embodiment, the immunogenic composition comprises an RV-C VP0 peptide.

In another preferred embodiment, the immunogenic composition comprises an RV-B VP0 peptide.

In another preferred embodiment, the immunogenic composition comprises RV-A28 VP0 and/or RV-A89 VP0.

Alternatively, in another embodiment, the immunogenic composition comprises at least two isolated human rhinovirus (RV) VP0 peptides, or isolated polynucleotides encoding the peptides, wherein the peptides are selected from a group consisting of: an RV-C VP0 peptide; an RV-B VP0 peptide; RV-A28 VP0; and RV-A89 VP0.

In another preferred embodiment, the immunogenic composition comprises at least three isolated human rhinovirus (RV) VP0 peptides, or isolated polynucleotides encoding the peptides, wherein the peptides are selected from a group consisting of: an RV-C VP0 peptide; an RV-B VP0 peptide; RV-A28 VP0; and RV-A89 VP0.

Preferably, the RV-C VP0 peptide is selected from a group consisting of: RV-C07 VP0; RV-C19 VP0; RV-C24 VP0; and RV-C01 VP0, or a variant or fragment thereof.

Preferably, the RV-B VP0 peptide is RV-B06 VP0.

Accordingly, in one embodiment, the immunogenic composition preferably comprises at least two isolated human rhinovirus (RV) VP0 peptides, or isolated polynucleotides encoding the peptides, wherein the peptides are selected from a group consisting of: RV-A28; RV-A89; RV-B06 VP0; RV-C07 VP0; RV-C19 VP0; RV-C24 VP0; and RV-C01 VP0, or a variant or fragment thereof.

Alternatively, the immunogenic composition preferably comprises at least three isolated human rhinovirus (RV) VP0 peptides, or isolated polynucleotides encoding the peptides, wherein the peptides are selected from a group consisting of: RV-A28; RV-A89; RV-B06 VP0; RV-C07 VP0; RV-C19 VP0; RV-C24 VP0; and RV-C01 VP0, or a variant or fragment thereof.

Alternatively, the immunogenic composition preferably comprises at least four isolated human rhinovirus (RV) VP0 peptides, or isolated polynucleotides encoding the peptides, wherein the peptides are selected from a group consisting of: RV-A28; RV-A89; RV-B06 VP0; RV-C07 VP0; RV-C19 VP0; RV-C24 VP0; and RV-C01 VP0, or a variant or fragment thereof.

Alternatively, the immunogenic composition comprises at least five isolated human rhinovirus (RV) VP0 peptides, or isolated polynucleotides encoding the peptides, wherein the peptides are selected from a group consisting of: RV-A28; RV-A89; RV-B06 VP0; RV-C07 VP0; RV-C19 VP0; RV-C24 VP0; and RV-C01 VP0, or a variant or fragment thereof.

Alternatively, the immunogenic composition comprises at least six isolated human rhinovirus (RV) VP0 peptides, or isolated polynucleotides encoding the peptides, wherein the peptides are selected from a group consisting of: RV-A28; RV-A89; RV-B06 VP0; RV-C07 VP0; RV-C19 VP0; RV-C24 VP0; and RV-C01 VP0, or a variant or fragment thereof.

Alternatively, the immunogenic composition comprises at least seven isolated human rhinovirus (RV) VP0 peptides, or isolated polynucleotides encoding the peptides, wherein the peptides are selected from a group consisting of: RV-A28; RV-A89; RV-B06 VP0; RV-C07 VP0; RV-C19 VP0; RV-C24 VP0; and RV-C01 VP0, or a variant or fragment thereof.

In one embodiment, the immunogenic composition may further comprise an isolated RV-A16 VP0 peptide and/or an isolated RV-A29 VP0 peptide, or an isolated polynucleotide encoding the peptide, or a variant or fragment thereof.

In a preferred embodiment, the immunogenic composition according to the first aspect comprises three isolated human rhinovirus (RV) VP0 peptides, or isolated polynucleotides encoding the peptides, wherein the peptides are selected from a group consisting of: RV-A16 VP0; RV-B06 VP0RV; and RV-C24 VP0, or a variant or fragment thereof.

It is well-known to the skilled person that there are three different species of rhinoviruses: rhinovirus A (RV-A), which is also called type A rhinovirus, rhinovirus B (RV-B), which is also called type B rhinovirus, and rhinovirus C (RV-C), which is also called type C rhinovirus. RVs are further classified into strains according to their nucleotide sequence homologies. As used herein, the term “strain” refers to a subdivision within a species of rhinoviruses and relies on the VP1 gene sequence of the rhinovirus. For example, the terms RV-A16, RV-A28, RV-A29, RV-A89, RV-B06, RV-C07, RV-C19, RV-C24, and RV-C01, refer to the RV strain. RVs have been classified according to several other parameters, including receptor specificity and antiviral susceptibility.

RVs have a 25 nm capsid of icosahedral symmetry, made up of 60 copies of each of four virus-coded proteins (VP1, VP2, VP3 and VP4) and enclosing a single-stranded RNA genome of approximately 7,500 nucleotides. The RNA is of positive polarity, is polyadenylated at its 3′ terminus and is covalently bound at its 5′ terminal end to a small protein, VPg. The primary translational product of this RNA is a single, “large” polyprotein, divided into three smaller polyproteins called, P1, P2 and P3, which are subsequently processed by proteolytic cleavage to yield the mature virus proteins. The P1 polyprotein is composed of four peptides (1A or VP4, 1B or VP2, 1C or VP3, and 1D or VP1), the P2 polyprotein is composed of three peptides (2A, 2B and 2C) and the P3 polyprotein is composed of four peptides (3A, 3B, 3C and 3D). 2A and 3C are viral proteases, while 3D corresponds to the viral RNA polymerase. The P1 polyprotein is the precursor that gives rise to the four structural proteins of the nucleocapsid. The P1 polyprotein is first cleaved to produce the VP0 polyprotein, which contains the amino acid sequence of VP4 and VP2 peptides, the VP3 peptide and the VP1 peptide. The VP0 polyprotein is then cleaved into the VP4 peptide and the VP2 peptide once the virus has assembled.

It will be well known to the skilled person that the term “VP0 polyprotein”, “VP0 peptide” or “peptide 1AB” refers to the protein precursor derived from the RV P1 polyprotein and which consists of the amino acid sequence of VP4 and VP2 peptides. VP0 polyprotein is typically about 330 amino acids long. The amino acid sequence of the VP0 polyprotein slightly varies according to the RV strain or species.

Accordingly, a fragment of the RV VP0 peptide may comprise or consist of the corresponding VP2 peptide and/or VP4 peptide. Thus, a fragment of the RV VP0 may have at least 1, 2, 3, 4, 5, 10, 20, 25, 30, 45, 50, 60, 70, 80, 90 or 100 amino acids fewer than the full length RV VP0 described herein.

In one embodiment, the peptide, variant or fragment thereof, comprises or consists of at least 40 amino acids, at least 45 amino acids, at least 50 amino acids, at least 55 amino acids, at least 60 amino acids, at least 65 amino acids, at least 66 amino acids, at least 67 amino acids, at least 68 amino acids, or at least 69 amino acids.

In another embodiment, the peptide, variant or fragment thereof, comprises or consists of at least 70 amino acids, at least 80 amino acids, at least 90 amino acids, at least 100 amino acids, at least 110 amino acids, at least 120 amino acids, at least 130 amino acids, at least 140 amino acids, at least 150 amino acids, at least 160 amino acids, at least 170 amino acids, at least 180 amino acids, at least 190 amino acids, at least 200 amino acids, at least 210 amino acids, at least 220 amino acids, at least 230 amino acids, at least 240 amino acids, at least 250 amino acids, at least 255 amino acids, at least 260 amino acids, at least 261 amino acids, at least 262 amino acids, at least 263 amino acids, at least 264 amino acids, at least 265 amino acids, at least 266 amino acids, or at least 267 amino acids.

In another embodiment, the peptide, variant or fragment thereof, comprises or consists of at least 280 amino acids, at least 290 amino acids, at least 300 amino acids, at least 310 amino acids, at least 320 amino acids, or at least 330 amino acids.

As used herein, the term “isolated” means removed from the natural environment, i.e. from rhinoviruses or cells infected by a rhinovirus. Usually, it refers to a peptide or a nucleic acid substantially free of cellular material, bacterial material, viral material, or culture medium when produced by recombinant DNA techniques, or chemical precursors, or other chemicals when chemically synthesized.

In one embodiment, at least two or more of the RV VP0 peptides may form a fusion peptide or protein. It is well-known to the skilled person that a fusion peptide/protein is a peptide comprising all or part of the amino acid sequence of at least two or more individual peptide units linked together via a covalent linkage, e.g. via peptide (amide) bonds. The two or more peptides may be joined directly to each other or they may be joined using a linker sequence. In another embodiment, at least three, four or five or more of the RV VP0 peptides may form a fusion peptide. Alternatively, a plurality of RV VP0 peptides may be kept as separate peptides, but used simultaneously, i.e. not fused together.

The present invention also relates to an immunogenic composition comprising an isolated polynucleotide(s) encoding the isolated human rhinovirus (RV) VP0 peptide(s).

In a preferred embodiment, the polynucleotide comprises a nucleic acid sequence encoding the peptide. Preferably, the nucleic acid sequence is placed under the control of the elements necessary for its expression in a mammalian cell, in particular in human cells. The nucleic acid sequence may be incorporated in a plasmid, which can be further formulated in a delivery vehicle such as liposomes to facilitate its introduction into the host cell.

As used herein, the term “nucleic acid” includes DNA and RNA, such as messenger RNA (mRNA) or self-amplifying RNA (saRNA), and can be either double-stranded or single-stranded. Most preferably, the nucleic acid is mRNA.

As used herein, the expression “elements necessary for expression in a mammalian cell” is understood to mean all the elements which allow the transcription of a DNA or DNA fragment into mRNA or saRNA and the translation of the latter into protein, inside a mammalian cell, such as a human cell. Typically, the elements necessary for the expression of a nucleic acid in a mammalian cell include a promoter that is functional in the selected mammalian cell and can be constitutive or inducible; a ribosome binding site; a start codon (ATG) if necessary; a region encoding a signal peptide (e.g., a lipidation signal peptide); a stop codon; and a 3′ terminal region (translation and/or transcription terminator). Other transcription control elements, such as enhancers, operators, and repressors can be also operatively associated with the polynucleotide to direct transcription and/or translation into the cell. The signal peptide-encoding region is preferably adjacent to the nucleic acid included in the immunogenic composition of the invention and placed in proper reading frame. The signal peptide-encoding region can be homologous or heterologous to the DNA molecule encoding the mature peptide or fusion peptide of the invention and can be specific to the secretion apparatus of the host used for expression. The open reading frame constituted by the nucleic acid included in the immunogenic composition of the invention, solely or together with the signal peptide, is placed under the control of the promoter so that transcription and translation occur in the host system. Promoters, (and signal peptide encoding regions) are widely known and available to those skilled in the art.

The nucleic acid sequences may be codon optimized such that the transcription of the DNA encoding the peptides and/or the fusion peptides of the invention is enhanced and/or the translation of the mRNA encoding the peptides and/or the fusion peptides is prolonged.

The immunogenic composition may comprise a concatemer of the isolated polynucleotides encoding the RV VP0 peptides. In a preferred embodiment, the polynucleotides encoding the RV VP0 peptides are mRNA. Accordingly, in one embodiment, the immunogenic composition comprises an isolated polynucleotide encoding at least two or more of the RV VP0 peptides. In another embodiment, the polynucleotide encodes at least three, four or five or more of the RV VP0 peptides. Alternatively, a plurality of RV VP0 peptides may be encoded by a plurality of separate polynucleotides, but used simultaneously, i.e. not fused together.

The inventors discovered that VP0 peptides from RV-A and RV-B species, elicited a stronger immune response when compared with VP0 peptides from RV-C groups. As such, the inventors believe that a concatemer in which the RV-C VP0 peptides are encoded first may be beneficial, as this will allow more of the RV-C VP0 peptides to be expressed. Accordingly, in one embodiment, the nucleic acid sequence encoding the RV-C VP0 peptide is disposed 5′ of the nucleic acid sequence encoding the RV-B VP0 peptide, the RV-A28 VP0 peptide and/or the RV-A89 VP0 peptide.

Alternatively, in another embodiment, the nucleic acid sequences encoding the RV-A VP0 peptide, the RV-B VP0 peptide, and the RV-C VP0 peptide are disposed in any order.

In one preferred embodiment, the immunogenic composition comprises an isolated polynucleotide encoding the RV-C24 VP0 peptide, an isolated polynucleotide encoding the RV-A16 VP0 peptide, and an isolated polynucleotide encoding the RV-B06 VP0 peptide. Preferably, the polynucleotides encoding the RV VP0 peptides are separated by a 2A peptide-encoding sequence.

In one embodiment, the amino acid sequence of the RV-A16 VP0 peptide is provided herein as SEQ ID No: 1, as follows:

[SEQ ID No: 1]
MGAQVSRQNVGTHSTQNMVSNGSSLNYFNINYFKDAASSGASRLD
FSQDPSKFTDPVKDVLEKGIPTLQSPSVEACGYSDRIIQITRGDS
TITSQDVANAVVGYGVWPHYLTPQDATAIDKPTQPDTSSNRFYTL
DSKMWNSTSKGWWWKLPDALKDMGIFGENMFYHFLGRSGYTVHVQ
CNASKFHQGTLLVVMIPEHQLATVNKGNVNAGYKYTHPGEAGREV
GTQVENEKQPSDDNWLNFDGTLLGNLLIFPHQFINLRSNNSATLI
VPYVNAVPMDSMVRHNNWSLVIIPVCQLQSNNISNIVPITVSISP
MCAEFSGARAKTVVQ

Accordingly, preferably the RV-A16 VP0 peptide comprises an amino acid sequence substantially as set out in SEQ ID No: 1, or a variant or fragment thereof.

The VP4 peptide of RV-A16 constitutes amino acid residues 1-69 of SEQ ID No: 1 and the VP2 peptide of RV-A16 constitutes amino acid residues 70-330 of SEQ ID No: 1. Thus, in embodiments in which the immunogenic composition comprises a fragment of RV-A16 VP0, the fragment may comprise or consist of the amino acids forming the VP2 and/or VP4 peptides.

In one embodiment, the RV-A16 VP0 peptide is encoded by the nucleotide sequence (DNA) of SEQ ID No: 2, as follows:

[SEQ ID No: 2]
ATGGGTGCGCAGGTGAGCCGTCAGAACGTGGGCACCCATAGCACC
CAGAACATGGTGAGCAACGGCAGCAGCCTGAACTATTTCAACATC
AACTATTTCAAAGATGCGGCGAGCAGCGGTGCGAGCCGTCTGGAT
TTCAGCCAGGATCCGAGCAAATTTACCGATCCGGTGAAAGATGTG
CTGGAAAAAGGCATTCCGACCCTGCAGAGCCCGAGCGTGGAAGCG
TGCGGCTATAGCGATCGTATTATTCAGATTACCCGTGGCGATAGC
ACCATTACCAGCCAGGATGTGGCGAACGCGGTGGTGGGCTATGGT
GTGTGGCCGCATTATCTGACCCCGCAGGATGCGACCGCGATTGAT
AAACCGACCCAGCCGGATACCAGCAGCAACCGTTTTTATACCCTG
GATAGCAAAATGTGGAACAGCACCAGCAAAGGTTGGTGGTGGAAA
CTGCCGGATGCGCTGAAAGATATGGGCATTTTCGGCGAAAACATG
TTCTATCATTTTCTGGGCCGTAGCGGCTATACCGTGCATGTGCAG
TGCAACGCGAGCAAATTTCATCAGGGCACCCTGCTGGTTGTGATG
ATTCCGGAACATCAGCTGGCCACCGTGAACAAAGGCAACGTGAAC
GCGGGCTATAAATATACCCATCCGGGCGAAGCGGGTCGTGAAGTG
GGCACCCAGGTGGAAAACGAAAAACAGCCGAGCGATGATAACTGG
CTGAACTTTGATGGCACCCTGCTGGGCAACCTGCTGATTTTTCCG
CATCAGTTCATTAACCTGCGTAGCAACAACAGCGCGACCCTGATT
GTGCCGTATGTGAACGCGGTGCCGATGGATAGCATGGTGCGTCAT
AACAATTGGAGCCTGGTGATTATTCCGGTGTGCCAGCTGCAGAGC
AACAACATTAGCAACATTGTGCCGATTACCGTGAGCATTAGCCCG
ATGTGCGCGGAATTTAGCGGTGCGCGTGCGAAAACCGTGGTGCAG

Accordingly, preferably the RV-A16 VP0 peptide is encoded by the nucleotide sequence substantially as set out in SEQ ID No: 2, or a variant or fragment thereof. Alternatively, the immunogenic composition comprises an isolated polynucleotide comprising the nucleotide sequence substantially as set out in SEQ ID No: 2, or a variant or fragment thereof.

In one embodiment, the RNA sequence corresponding to the DNA sequence of SEQ ID No: 2 is provided herein as SEQ ID No: 3, as follows:

[SEQ ID No: 3]
AUGGGUGCGCAGGUGAGCCGUCAGAACGUGGGCACCCAUAGCACC
CAGAACAUGGUGAGCAACGGCAGCAGCCUGAACUAUUUCAACAUC
AACUAUUUCAAAGAUGCGGCGAGCAGCGGUGCGAGCCGUCUGGAU
UUCAGCCAGGAUCCGAGCAAAUUUACCGAUCCGGUGAAAGAUGUG
CUGGAAAAAGGCAUUCCGACCCUGCAGAGCCCGAGCGUGGAAGCG
UGCGGCUAUAGCGAUCGUAUUAUUCAGAUUACCCGUGGCGAUAGC
ACCAUUACCAGCCAGGAUGUGGCGAACGCGGUGGUGGGCUAUGGU
GUGUGGCCGCAUUAUCUGACCCCGCAGGAUGCGACCGCGAUUGAU
AAACCGACCCAGCCGGAUACCAGCAGCAACCGUUUUUAUACCCUG
GAUAGCAAAAUGUGGAACAGCACCAGCAAAGGUUGGUGGUGGAAA
CUGCCGGAUGCGCUGAAAGAUAUGGGCAUUUUCGGCGAAAACAUG
UUCUAUCAUUUUCUGGGCCGUAGCGGCUAUACCGUGCAUGUGCAG
UGCAACGCGAGCAAAUUUCAUCAGGGCACCCUGCUGGUUGUGAUG
AUUCCGGAACAUCAGCUGGCCACCGUGAACAAAGGCAACGUGAAC
GCGGGCUAUAAAUAUACCCAUCCGGGCGAAGCGGGUCGUGAAGUG
GGCACCCAGGUGGAAAACGAAAAACAGCCGAGCGAUGAUAACUGG
CUGAACUUUGAUGGCACCCUGCUGGGCAACCUGCUGAUUUUUCCG
CAUCAGUUCAUUAACCUGCGUAGCAACAACAGCGCGACCCUGAUU
GUGCCGUAUGUGAACGCGGUGCCGAUGGAUAGCAUGGUGCGUCAU
AACAAUUGGAGCCUGGUGAUUAUUCCGGUGUGCCAGCUGCAGAGC
AACAACAUUAGCAACAUUGUGCCGAUUACCGUGAGCAUUAGCCCG
AUGUGCGCGGAAUUUAGCGGUGCGCGUGCGAAAACCGUGGUGCAG

Accordingly, in one embodiment, the immunogenic composition comprises an isolated polynucleotide comprising the nucleotide sequence (RNA) substantially as set out in SEQ ID No: 3, or a variant or fragment thereof.

In one embodiment, the amino acid sequence of the RV-B06 VP0 peptide is provided herein as SEQ ID No: 4, as follows:

[SEQ ID No: 4]
MGAQVSTQKSGSHENQNILINGSNQTFTVINYYKDAASSSSAGQS
FSTDPSKFTEPVKDLMLKGAPALNSPNVEACGYSDRVQQITLGNS
TITTQEAANAVVCYAEWPEYLPDGDASDVNKTSKPDTSVCRFYTL
ESKTWQNSSKGWCWKLPDALKDMGVFGQNMFFHSLGRSGYTIHVQ
CNATKFHSGCLLVVVIPEHQLASHKGGNVSVKYKYTHPGESGIDL
STQLDEDGPVKDPVYSMNGTLLGNLLIFPHQFINLRINNTATIVV
PYINSVPIDSMTRHNNVSLLVIPIAPLVAPTGSPQTLPVTVTIAP
MCTEFTGIRSKTIVPQ

Accordingly, preferably the RV-B06 VP0 peptide comprises an amino acid sequence substantially as set out in SEQ ID No: 4, or a variant or fragment thereof.

The VP4 peptide of RV-B06 constitutes amino acid residues 1-69 of SEQ ID No: 4 and the VP2 peptide of RV-B06 constitutes amino acid residues 70-331 of SEQ ID No: 4. Thus, in embodiments in which the immunogenic composition comprises a fragment of RV-B06 VP0, the fragment may comprise or consist of the amino acids forming the VP2 and/or VP4 peptides.

In one embodiment, the RV-B06 VP0 peptide is encoded by the nucleotide sequence (DNA) of SEQ ID No: 5, as follows:

[SEQ ID No: 5]
ATGGGCGCTCAAGTTTCAACACAAAAGAGTGGTTCTCACGAGAAT
CAGAACATCTTAACTAATGGCTCAAATCAGACGTTCACAGTTATA
AATTATTATAAAGATGCAGCTAGCTCATCATCAGCAGGTCAGTCT
TTCTCAACGGACCCATCAAAATTCACAGAACCAGTTAAAGACCTG
ATGTTGAAAGGTGCCCCAGCATTGAACTCACCAAATGTTGAAGCA
TGTGGTTATAGTGACAGAGTCCAACAAATTACTCTAGGTAATTCC
ACTATAACAACCCAGGAAGCAGCCAATGCGGTTGTGTGTTATGCT
GAGTGGCCTGAATACTTGCCAGATGGTGATGCTAGTGATGTTAAT
AAGACTTCCAAACCAGACACATCTGTATGTAGATTCTACACATTG
GAGAGTAAAACATGGCAAAACAGCTCAAAGGGATGGTGTTGGAAA
CTACCAGATGCTCTCAAGGATATGGGTGTGTTTGGGCAAAACATG
TTCTTCCACTCGCTTGGTAGATCTGGCTATACTATACATGTGCAA
TGTAATGCAACTAAATTCCATAGTGGCTGTCTTCTTGTTGTTGTT
ATCCCAGAACACCAATTGGCATCTCACAAAGGCGGGAATGTGTCA
GTGAAGTATAAATATACACATCCAGGAGAGAGTGGTATAGACTTG
AGTACACAGTTAGATGAGGATGGTCCAGTGAAAGATCCTGTCTAT
AGCATGAATGGCACTCTATTAGGTAACTTACTCATCTTCCCTCAC
CAGTTTATCAACCTCAGAACAAACAATACAGCCACAATAGTTGTG
CCATATATTAATTCAGTGCCAATAGACTCAATGACACGCCATAAT
AACGTGTCACTCTTAGTGATTCCCATAGCACCTCTAGTGGCTCCA
ACTGGATCACCACAAACACTTCCAGTGACAGTGACTATAGCTCCC
ATGTGTACAGAGTTCACGGGTATTAGATCAAAAACCATTGTTCCA
CAG

Accordingly, preferably the RV-B06 VP0 peptide is encoded by the nucleotide sequence substantially as set out in SEQ ID No: 5, or a variant or fragment thereof. Alternatively, the immunogenic composition comprises an isolated polynucleotide comprising the nucleotide sequence substantially as set out in SEQ ID No: 5, or a variant or fragment thereof.

In one embodiment, the RNA sequence corresponding to the DNA sequence of SEQ ID No: 5 is provided herein as SEQ ID No: 6, as follows:

[SEQ ID No: 6]
AUGGGCGCUCAAGUUUCAACACAAAAGAGUGGUUCUCACGAGAAU
CAGAACAUCUUAACUAAUGGCUCAAAUCAGACGUUCACAGUUAUA
AAUUAUUAUAAAGAUGCAGCUAGCUCAUCAUCAGCAGGUCAGUCU
UUCUCAACGGACCCAUCAAAAUUCACAGAACCAGUUAAAGACCUG
AUGUUGAAAGGUGCCCCAGCAUUGAACUCACCAAAUGUUGAAGCA
UGUGGUUAUAGUGACAGAGUCCAACAAAUUACUCUAGGUAAUUCC
ACUAUAACAACCCAGGAAGCAGCCAAUGCGGUUGUGUGUUAUGCU
GAGUGGCCUGAAUACUUGCCAGAUGGUGAUGCUAGUGAUGUUAAU
AAGACUUCCAAACCAGACACAUCUGUAUGUAGAUUCUACACAUUG
GAGAGUAAAACAUGGCAAAACAGCUCAAAGGGAUGGUGUUGGAAA
CUACCAGAUGCUCUCAAGGAUAUGGGUGUGUUUGGGCAAAACAUG
UUCUUCCACUCGCUUGGUAGAUCUGGCUAUACUAUACAUGUGCAA
UGUAAUGCAACUAAAUUCCAUAGUGGCUGUCUUCUUGUUGUUGUU
AUCCCAGAACACCAAUUGGCAUCUCACAAAGGCGGGAAUGUGUCA
GUGAAGUAUAAAUAUACACAUCCAGGAGAGAGUGGUAUAGACUUG
AGUACACAGUUAGAUGAGGAUGGUCCAGUGAAAGAUCCUGUCUAU
AGCAUGAAUGGCACUCUAUUAGGUAACUUACUCAUCUUCCCUCAC
CAGUUUAUCAACCUCAGAACAAACAAUACAGCCACAAUAGUUGUG
CCAUAUAUUAAUUCAGUGCCAAUAGACUCAAUGACACGCCAUAAU
AACGUGUCACUCUUAGUGAUUCCCAUAGCACCUCUAGUGGCUCCA
ACUGGAUCACCACAAACACUUCCAGUGACAGUGACUAUAGCUCCC
AUGUGUACAGAGUUCACGGGUAUUAGAUCAAAAACCAUUGUUCCA
CAG

Accordingly, in one embodiment, the immunogenic composition comprises an isolated polynucleotide comprising the nucleotide sequence (RNA) substantially as set out in SEQ ID No: 6, or a variant or fragment thereof.

In one embodiment, the amino acid sequence of the RV-C07 VP0 peptide is provided herein as SEQ ID No: 7, as follows:

[SEQ ID No: 7]
MGAQVSKQSVGAHETMVHAGSGAVVKYFNINYYKDAASSGLTKQD
FSQDPSKFTQPVADLLINPALMSPSVEACGYSDRLKQITIGSSTI
TTQDSVNTIVAYGEWPSYLSDLDASSVDKPTHPETSADRFYTLDS
VRWGGSSKGWWWKLPDCLKNMGIFGQNMYYHAMGRSGYIIHTQCN
ATKFHSGCLLVAVVPEHQLAYIGGTNAQVSYKHTHPGERGHEIGL
NTSRGDNRPDEDPFFNCNGTLLGNLTIFPHQLINLRINNSSTIVV
PYINCTPMDSMLRHNNVSLVIIPICPLRTPSGAPTTLPITVSIAP
DRSEFSGARQSATRQ

Accordingly, preferably the RV-C07VP0 peptide comprises an amino acid sequence substantially as set out in SEQ ID No: 7, or a variant or fragment thereof.

The VP4 peptide of RV-C07 constitutes amino acid residues 1-67 of SEQ ID No: 7 and the VP2 peptide of RV-C07 constitutes amino acid residues 68-330 of SEQ ID No: 7. Thus, in embodiments in which the immunogenic composition comprises a fragment of RV-C07 VP0, the fragment may comprise or consist of the amino acids forming the VP2 and/or VP4 peptides.

In one embodiment, the RV-C07 VP0 peptide is encoded by the nucleotide sequence (DNA) of SEQ ID No: 8, as follows:

[SEQ ID No: 8]
ATGGGCGCTCAGGTTTCCAAGCAGAGTGTGGGAGCTCATGAAACT
ATGGTGCATGCTGGCTCTGGTGCAGTTGTTAAATACTTCAACATC
AATTATTACAAGGATGCTGCTAGCTCCGGTTTGACCAAACAAGAT
TTCTCACAAGACCCATCCAAGTTTACTCAGCCTGTAGCAGATTTA
CTGACAAATCCAGCTTTGATGTCCCCATCAGTTGAGGCGTGTGGA
TACTCCGATAGGCTCAAGCAAATCACTATCGGAAGCTCCACCATT
ACAACACAAGATTCTGTCAACACCATAGTTGCCTATGGCGAGTGG
CCTAGITATTTGTCAGATTTAGATGCATCATCTGTTGATAAACCC
ACACATCCTGAAACATCGGCTGACAGATTCTACACTTTGGACAGC
GTACGCTGGGGTGGTAGTTCCAAAGGATGGTGGTGGAAGTTGCCA
GACTGITTGAAGAACATGGGTATATTTGGGCAGAACATGTACTAC
CACGCTATGGGAAGGTCTGGTTACATCATACACACCCAGTGCAAT
GCCACTAAGTTCCACAGTGGATGTCTGTTGGTAGCGGTTGTTCCC
GAACACCAGCTAGCTTACATTGGGGGTACCAATGCACAAGTTAGC
TACAAACATACTCACCCAGGTGAACGTGGGCATGAAATAGGTCTC
AACACAAGCAGAGGTGACAACCGGCCAGATGAGGATCCCTTTTTT
AATTGCAACGGAACACTATTAGGTAACTTGACTATTTTCCCTCAC
CAGCTCATAAACCTGAGGACAAATAACTCCAGCACCATAGITGTG
CCTTACATTAACTGCACACCAATGGACAGTATGTTACGCCACAAC
AATGTCTCTCTTGTAATTATACCCATCTGTCCTTTACGAACTCCC
AGTGGAGCACCCACTACCTTACCAATTACTGTTTCAATTGCCCCA
GACAGATCTGAATTCTCTGGCGCCAGGCAGTCCGCCACCCGCCAG

Accordingly, preferably the RV-C07 VP0 peptide is encoded by the nucleotide sequence substantially as set out in SEQ ID No: 8, or a variant or fragment thereof. Alternatively, the immunogenic composition comprises an isolated polynucleotide comprising the nucleotide sequence substantially as set out in SEQ ID No: 8, or a variant or fragment thereof.

In one embodiment, the RNA sequence corresponding to the DNA sequence of SEQ ID No: 8 is provided herein as SEQ ID No: 9, as follows:

[SEQ ID No: 9]
AUGGGCGCUCAGGUUUCCAAGCAGAGUGUGGGAGCUCAUGAAACU
AUGGUGCAUGCUGGCUCUGGUGCAGUUGUUAAAUACUUCAACAUC
AAUUAUUACAAGGAUGCUGCUAGCUCCGGUUUGACCAAACAAGAU
UUCUCACAAGACCCAUCCAAGUUUACUCAGCCUGUAGCAGAUUUA
CUGACAAAUCCAGCUUUGAUGUCCCCAUCAGUUGAGGCGUGUGGA
UACUCCGAUAGGCUCAAGCAAAUCACUAUCGGAAGCUCCACCAUU
ACAACACAAGAUUCUGUCAACACCAUAGUUGCCUAUGGCGAGUGG
CCUAGUUAUUUGUCAGAUUUAGAUGCAUCAUCUGUUGAUAAACCC
ACACAUCCUGAAACAUCGGCUGACAGAUUCUACACUUUGGACAGC
GUACGCUGGGGUGGUAGUUCCAAAGGAUGGUGGUGGAAGUUGCCA
GACUGUUUGAAGAACAUGGGUAUAUUUGGGCAGAACAUGUACUAC
CACGCUAUGGGAAGGUCUGGUUACAUCAUACACACCCAGUGCAAU
GCCACUAAGUUCCACAGUGGAUGUCUGUUGGUAGCGGUUGUUCCC
GAACACCAGCUAGCUUACAUUGGGGGUACCAAUGCACAAGUUAGC
UACAAACAUACUCACCCAGGUGAACGUGGGCAUGAAAUAGGUCUC
AACACAAGCAGAGGUGACAACCGGCCAGAUGAGGAUCCCUUUUUU
AAUUGCAACGGAACACUAUUAGGUAACUUGACUAUUUUCCCUCAC
CAGCUCAUAAACCUGAGGACAAAUAACUCCAGCACCAUAGUUGUG
CCUUACAUUAACUGCACACCAAUGGACAGUAUGUUACGCCACAAC
AAUGUCUCUCUUGUAAUUAUACCCAUCUGUCCUUUACGAACUCCC
AGUGGAGCACCCACUACCUUACCAAUUACUGUUUCAAUUGCCCCA
GACAGAUCUGAAUUCUCUGGCGCCAGGCAGUCCGCCACCCGCCAG

Accordingly, in one embodiment, the immunogenic composition comprises an isolated polynucleotide comprising the nucleotide sequence (RNA) substantially as set out in SEQ ID No: 9, or a variant or fragment thereof.

In one embodiment, the amino acid sequence of the RV-C19 VP0 peptide is provided herein as SEQ ID No: 10, as follows:

[SEQ ID No: 10]
MGAQVSKQNVGSHENSVSASNGSVIKYFNINYYKDSASSGLTKQD
FSQDPSKFTQPLVDTLTNPALMSPSVEACGFSDRLKQITIGNSTI
TTQDSLNTVLAYGEWPSYLSDLDATSVDKPSHPETSSDRFYTLES
VDWKSGSKGWWWKFPDALKDMGMFGQNMYHHSMGRFGALIHVQCN
ATKFHSGCLLIMVVPEHQLAYIGAEGVKVRYEHTHPGEKGHTLKG
SQDRSINNPDDNPFYMCNGTLLGNGMLYPHQMINLRINNSATIVI
PYINCVPMDNMLRHNNFSLVIVPIVPLRPGNSGAPILPITVTIAP
YKSEFSGAMKSQPNQ

Accordingly, preferably the RV-C19 VP0 peptide comprises an amino acid sequence substantially as set out in SEQ ID No: 10, or a variant or fragment thereof.

The VP4 peptide of RV-C19 constitutes amino acid residues 1-67 of SEQ ID No: 10 and the VP2 peptide of RV-C19 constitutes amino acid residues 68-330 of SEQ ID No: 10. Thus, in embodiments in which the immunogenic composition comprises a fragment of RV-C19 VP0, the fragment may comprise or consist of the amino acids forming the VP2 and/or VP4 peptides.

In one embodiment, the RV-C19 VP0 peptide is encoded by the nucleotide sequence (DNA) of SEQ ID No: 11, as follows:

[SEQ ID No: 11]
ATGGGTGCACAGGTCAGCAAACAGAATGTTGGATCACACGAAAAC
TCTGTGTCTGCTAGTAATGGCTCTGTAATCAAGTACTTTAACATC
AATTACTATAAAGACTCTGCCAGCTCTGGGTTGACTAAACAAGAC
TTTTCACAAGACCCGTCCAAATTTACTCAACCACTCGTTGACACA
CTGACAAATCCCGCCTTGATGTCCCCAAGTGTTGAAGCTTGTGGG
TTTTCCGATAGGCTTAAACAAATCACTATCGGAAACTCCACTATT
ACAACACAAGATTCGCTCAACACCGTCTTGGCGTATGGAGAATGG
CCTTCATACCTCTCTGACCTAGATGCCACCTCAGTAGATAAACCA
TCACACCCAGAAACCTCCTCAGACCGATTCTATACACTAGAAAGT
GTTGATTGGAAGTCCGGATCTAAAGGTTGGTGGTGGAAATTTCCA
GATGCCCTAAAAGATATGGGAATGTTTGGGCAAAACATGTACCAC
CATTCAATGGGTAGGTTTGGAGCTCTCATACATGTCCAATGTAAT
GCTACTAAATTTCACAGTGGTTGTCTCCTGATCATGGTTGTCCCC
GAACATCAATTAGCATATATAGGGGCAGAAGGTGTCAAGGTTAGA
TATGAGCACACACACCCAGGAGAAAAGGGCCATACCTTGAAAGGT
AGCCAGGATAGATCAACTAACAATCCAGATGATAACCCATTCTAC
ATGTGTAACGGTACACTACTGGGTAATGGTATGTTATATCCTCAT
CAGATGATCAACCTCCGCACCAACAACTCAGCCACTATTGTAATT
CCATACATCAACTGTGTACCCATGGACAATATGTTGCGCCACAAC
AACTTCAGTCTGGTGATTGTACCAATAGTACCCTTGAGACCTGGG
AACTCAGGAGCTCCTATCTTACCCATCACTGTGACCATAGCACCA
TACAAATCAGAGTTTTCTGGAGCTATGAAATCCCAACCCAATCAG

Accordingly, preferably the RV-C19 VP0 peptide is encoded by the nucleotide sequence substantially as set out in SEQ ID No: 11, or a variant or fragment thereof. Alternatively, the immunogenic composition comprises an isolated polynucleotide comprising the nucleotide sequence substantially as set out in SEQ ID No: 11, or a variant or fragment thereof.

In one embodiment, the RNA sequence corresponding to the DNA sequence of SEQ ID No: 11 is provided herein as SEQ ID No: 12, as follows:

[SEQ ID No: 12]
AUGGGUGCACAGGUCAGCAAACAGAAUGUUGGAUCACACGAAAACUCUGUGUCUGCUAGUAAUGGCUCUG
UAAUCAAGUACUUUAACAUCAAUUACUAUAAAGACUCUGCCAGCUCUGGGUUGACUAAACAAGACUUUUC
ACAAGACCCGUCCAAAUUUACUCAACCACUCGUUGACACACUGACAAAUCCCGCCUUGAUGUCCCCAAGU
GUUGAAGCUUGUGGGUUUUCCGAUAGGCUUAAACAAAUCACUAUCGGAAACUCCACUAUUACAACACAAG
AUUCGCUCAACACCGUCUUGGCGUAUGGAGAAUGGCCUUCAUACCUCUCUGACCUAGAUGCCACCUCAGU
AGAUAAACCAUCACACCCAGAAACCUCCUCAGACCGAUUCUAUACACUAGAAAGUGUUGAUUGGAAGUCC
GGAUCUAAAGGUUGGUGGUGGAAAUUUCCAGAUGCCCUAAAAGAUAUGGGAAUGUUUGGGCAAAACAUGU
ACCACCAUUCAAUGGGUAGGUUUGGAGCUCUCAUACAUGUCCAAUGUAAUGCUACUAAAUUUCACAGUGG
UUGUCUCCUGAUCAUGGUUGUCCCCGAACAUCAAUUAGCAUAUAUAGGGGCAGAAGGUGUCAAGGUUAGA
UAUGAGCACACACACCCAGGAGAAAAGGGCCAUACCUUGAAAGGUAGCCAGGAUAGAUCAACUAACAAUC
CAGAUGAUAACCCAUUCUACAUGUGUAACGGUACACUACUGGGUAAUGGUAUGUUAUAUCCUCAUCAGAU
GAUCAACCUCCGCACCAACAACUCAGCCACUAUUGUAAUUCCAUACAUCAACUGUGUACCCAUGGACAAU
AUGUUGCGCCACAACAACUUCAGUCUGGUGAUUGUACCAAUAGUACCCUUGAGACCUGGGAACUCAGGAG
CUCCUAUCUUACCCAUCACUGUGACCAUAGCACCAUACAAAUCAGAGUUUUCUGGAGCUAUGAAAUCCCA
ACCCAAUUAACAG

Accordingly, in one embodiment, the immunogenic composition comprises an isolated polynucleotide comprising the nucleotide sequence (RNA) substantially as set out in SEQ ID No: 12, or a variant or fragment thereof.

In one embodiment, the amino acid sequence of the RV-C24 VP0 peptide is provided herein as SEQ ID No: 13, as follows:

[SEQ ID No: 13]
MGAQVSKQNVGSHENSVSATGGSVIKYFNINYYKDSASSGLTKQD
FSQDPSKFTQPLAEALTNPALMSPTVEACGMSDRLKQITIGNSTI
TTQDTLNSILAYGEWPKYLSDLDASSVDKPTHPETSSDRFYTLTS
VDWTTTSKGWWWKLPDCLKDMGIFGQNLYHHALGRSGYIIHTQCN
ATKENSGCLIVAVVPEHQLAYIGEANVNVGYDHTHPGEGGHVIGS
NVRRDNKQPDEDPFFNCNGTLLGNITIFPHQLINLRINNSSTIVV
PYINCVPMDNMLRHNNLSLVIIPIVPLRAANGVTKVPITISIAPD
KSEFSGARQSVKQ

Accordingly, preferably the RV-C24 VP0 peptide comprises an amino acid sequence substantially as set out in SEQ ID No: 13, or a variant or fragment thereof.

The VP4 peptide of RV-C24 constitutes amino acid residues 1-67 of SEQ ID No: 13 and the VP2 peptide of RV-C24 constitutes amino acid residues 68-328 of SEQ ID No: 13. Thus, in embodiments in which the immunogenic composition comprises a fragment of RV-C24 VP0, the fragment may comprise or consist of the amino acids forming the VP2 and/or VP4 peptides.

In one embodiment, the RV-C24 VP0 peptide is encoded by the nucleotide sequence (DNA) of SEQ ID No: 14, as follows:

[SEQ ID No: 14]
ATGGGCGCACAGGTCAGCAAGCAAAATGTCGGCTCGCATGAAAAC
TCAGTCTCAGCCACAGGTGGATCCGTGATTAAGTATTTCAACATC
AATTACTACAAGGATTCTGCTAGCTCTGGCTTGACTAAACAAGAT
TTTTCCCAAGACCCATCGAAATTCACACAACCTCTAGCAGAAGCA
CTTACAAATCCAGCTTTAATGTCACCAACTGTTGAAGCATGTGGG
ATGTCCGATAGGCTTAAACAAATTACTATCGGGAATTCCACTATA
ACAACACAAGATACACTAAACTCTATACTGGCATATGGGGAGTGG
CCCAAATACTTGAGTGACCTGGACGCTTCCTCAGTGGATAAACCT
ACCCACCCAGAAACATCATCTGATAGATTTTACACATTAACTAGT
GTAGATTGGACCACTACGTCTAAAGGTTGGTGGTGGAAGTTGCCT
GATTGCCTTAAAGATATGGGCATCTTCGGGCAAAATCTGTACCAT
CATGCATTGGGTAGGTCAGGGTACATAATACACACCCAATGTAAT
GCCACAAAATTCAATAGTGGTTGTCTAATAGTGGCTGTTGTACCA
GAACACCAGCTAGCTTACATAGGTGAAGCAAATGTCAATGTTGGT
TATGATCACACACACCCTGGTGAGGGAGGACATGTAATTGGTTCA
AATGTTAGGAGAGATAACAAGCAACCTGATGAAGACCCCTTCTTT
AATTGTAATGGGACCCTGCTTGGTAACATCACTATATTCCCACAC
CAGCTCATAAACTTGAGGACAAACAATTCCAGCACAATTGTTGTA
CCATACATTAATTGTGTACCTATGGACAACATGCTCAGGCACAAC
AACCTATCTCTAGTTATTATTCCAATCGTTCCTCTCAGAGCCGCA
AATGGTGTCACCAAGGTCCCCATTACAATCTCAATAGCACCAGAT
AAGTCAGAGTTCTCAGGGGCTAGACAGTCTGTAAAACAG

Accordingly, preferably the RV-C24 VP0 peptide is encoded by the nucleotide sequence substantially as set out in SEQ ID No: 14, or a variant or fragment thereof. Alternatively, the immunogenic composition comprises an isolated polynucleotide comprising the nucleotide sequence substantially as set out in SEQ ID No: 14, or a variant or fragment thereof.

In one embodiment, the RNA sequence corresponding to the DNA sequence of SEQ ID No: 14 is provided herein as SEQ ID No: 15, as follows:

[SEQ ID No: 15]
AUGGGCGCACAGGUCAGCAAGCAAAAUGUCGGCUCGCAUGAAAAC
UCAGUCUCAGCCACAGGUGGAUCCGUGAUUAAGUAUUUCAACAUC
AAUUACUACAAGGAUUCUGCUAGCUCUGGCUUGACUAAACAAGAU
UUUUCCCAAGACCCAUCGAAAUUCACACAACCUCUAGCAGAAGCA
CUUACAAAUCCAGCUUUAAUGUCACCAACUGUUGAAGCAUGUGGG
AUGUCCGAUAGGCUUAAACAAAUUACUAUCGGGAAUUCCACUAUA
ACAACACAAGAUACACUAAACUCUAUACUGGCAUAUGGGGAGUGG
CCCAAAUACUUGAGUGACCUGGACGCUUCCUCAGUGGAUAAACCU
ACCCACCCAGAAACAUCAUCUGAUAGAUUUUACACAUUAACUAGU
GUAGAUUGGACCACUACGUCUAAAGGUUGGUGGUGGAAGUUGCCU
GAUUGCCUUAAAGAUAUGGGCAUCUUCGGGCAAAAUCUGUACCAU
CAUGCAUUGGGUAGGUCAGGGUACAUAAUACACACCCAAUGUAAU
GCCACAAAAUUCAAUAGUGGUUGUCUAAUAGUGGCUGUUGUACCA
GAACACCAGCUAGCUUACAUAGGUGAAGCAAAUGUCAAUGUUGGU
UAUGAUCACACACACCCUGGUGAGGGAGGACAUGUAAUUGGUUCA
AAUGUUAGGAGAGAUAACAAGCAACCUGAUGAAGACCCCUUCUUU
AAUUGUAAUGGGACCCUGCUUGGUAACAUCACUAUAUUCCCACAC
CAGCUCAUAAACUUGAGGACAAACAAUUCCAGCACAAUUGUUGUA
CCAUACAUUAAUUGUGUACCUAUGGACAACAUGCUCAGGCACAAC
AACCUAUCUCUAGUUAUUAUUCCAAUCGUUCCUCUCAGAGCCGCA
AAUGGUGUCACCAAGGUCCCCAUUACAAUCUCAAUAGCACCAGAU
AAGUCAGAGUUCUCAGGGGCUAGACAGUCUGUAAAACAGCAG

Accordingly, in one embodiment, the immunogenic composition comprises an isolated polynucleotide comprising the nucleotide sequence (RNA) substantially as set out in SEQ ID No: 15, or a variant or fragment thereof.

In one embodiment, the amino acid sequence of the RV-C01VP0 peptide is provided herein as SEQ ID No: 16, as follows:

[SEQ ID No: 16]
MGAQVSRQSVGSHETMIHAGTGAVVKYFNVNYYKDAASSGLTKQD
FSQDPSKFTQPVADILINPALMSPSVEACGFSDRLKQITIGNSTI
TTQDAVNTIVAYGEWPSYLSDLDATSVDKPTHPETSSDRFYTLRS
VDWRGGSKGWWWKLPDCLKDMGIFGQNMFHHAMGRSGYIIHTQCN
ATKFHSGCLLVAVVPEHQLAYIGADARVSYEHTHPGERGHTIGSN
ADRNNHQPDENPFFNCNGTLLGNLTIFPHQLINLRINNSSTIVVP
YINCTPMDSMLRHNNVSLVIIPICPLRPPTTGQQVLPITISIAPI
KSEFSGARQAIKQ

Accordingly, preferably the RV-C01VP0 peptide comprises an amino acid sequence substantially as set out in SEQ ID No: 16, or a variant or fragment thereof.

The VP4 peptide of RV-C01 constitutes amino acid residues 1-67 of SEQ ID No: 16 and the VP2 peptide of RV-C01 constitutes amino acid residues 68-328 of SEQ ID No: 16. Thus, in embodiments in which the immunogenic composition comprises a fragment of RV-C01 VP0, the fragment may comprise or consist of the amino acids forming the VP2 and/or VP4 peptides.

In one embodiment, the RV-C01VP0 peptide is encoded by the nucleotide sequence (DNA) of SEQ ID No: 17, as follows:

[SEQ ID No: 17]
ATGGGCGCACAGGTGTCTAGACAGAGTGTCGGGAGCCATGAAACC
ATGATCCATGCTGGGACTGGAGCCGTAGTAAAGTACTTCAATGTC
AATTATTACAAAGATGCAGCAAGCTCTGGATTGACCAAGCAAGAT
TTCTCCCAGGATCCATCCAAATTTACTCAACCCGTGGCCGACATC
CTGACCAACCCAGCTTTGATGTCCCCCTCCGTTGAAGCTTGTGGG
TTTTCCGATAGGCTCAAGCAAATCACTATTGGAAACTCCACTATA
ACAACCCAAGATGCAGTCAATACAATTGTGGCATACGGGGAATGG
CCCAGTTATCTATCAGATTTGGATGCGACTTCAGTTGATAAACCG
ACGCACCCTGAGACATCATCAGACAGATTTTACACGCTGAGGAGT
GTTGACTGGCGAGGTGGTTCAAAAGGGTGGTGGTGGAAACTGCCT
GATTGTCTGAAAGACATGGGAATATTTGGGCAAAACATGTTCCAC
CATGCCATGGGAAGATCAGGATACATCATACACACCCAGTGCAAT
GCAACTAAATTTCACAGTGGATGTTTACTCGTGGCAGTAGTGCCT
GAACACCAATTAGCATACATAGGTGCAGATGCACGAGTGAGTTAT
GAGCACACCCACCCTGGTGAGCGAGGCCATACAATTGGTAGCAAC
GCAGACAGGAATAACCATCAGCCAGATGAAAATCCTTTCTTCAAC
TGTAACGGGACTTTACTTGGAAATTTGACTATCTTCCCACACCAG
CTTATAAATTTAAGGACAAACAACTCAAGCACCATCGTCGTGCCT
TACATCAATTGCACTCCTATGGATAGCATGTTACGGCACAACAAT
GTATCACTTGTTATCATACCCATTTGCCCTTTACGGCCCCCAACC
ACAGGGCAGCAAGTCTTACCGATCACCATTTCTATTGCTCCCATA
AAATCAGAGTTTTCAGGGGCTAGGCAGGCCATTAAACAA

Accordingly, preferably the RV-C01VP0 peptide is encoded by the nucleotide sequence substantially as set out in SEQ ID No: 17, or a variant or fragment thereof. Alternatively, the immunogenic composition comprises an isolated polynucleotide comprising the nucleotide sequence substantially as set out in SEQ ID No: 17, or a variant or fragment thereof.

In one embodiment, the RNA sequence corresponding to the DNA sequence of SEQ ID No: 17 is provided herein as SEQ ID No: 18, as follows:

[SEQ ID No: 18]
AUGGGCGCACAGGUGUCUAGACAGAGUGUCGGGAGCCAUGAAACC
AUGAUCCAUGCUGGGACUGGAGCCGUAGUAAAGUACUUCAAUGUC
AAUUAUUACAAAGAUGCAGCAAGCUCUGGAUUGACCAAGCAAGAU
UUCUCCCAGGAUCCAUCCAAAUUUACUCAACCCGUGGCCGACAUC
CUGACCAACCCAGCUUUGAUGUCCCCCUCCGUUGAAGCUUGUGGG
UUUUCCGAUAGGCUCAAGCAAAUCACUAUUGGAAACUCCACUAUA
ACAACCCAAGAUGCAGUCAAUACAAUUGUGGCAUACGGGGAAUGG
CCCAGUUAUCUAUCAGAUUUGGAUGCGACUUCAGUUGAUAAACCG
ACGCACCCUGAGACAUCAUCAGACAGAUUUUACACGCUGAGGAGU
GUUGACUGGCGAGGUGGUUCAAAAGGGUGGUGGUGGAAACUGCCU
GAUUGUCUGAAAGACAUGGGAAUAUUUGGGCAAAACAUGUUCCAC
CAUGCCAUGGGAAGAUCAGGAUACAUCAUACACACCCAGUGCAAU
GCAACUAAAUUUCACAGUGGAUGUUUACUCGUGGCAGUAGUGCCU
GAACACCAAUUAGCAUACAUAGGUGCAGAUGCACGAGUGAGUUAU
GAGCACACCCACCCUGGUGAGCGAGGCCAUACAAUUGGUAGCAAC
GCAGACAGGAAUAACCAUCAGCCAGAUGAAAAUCCUUUCUUCAAC
UGUAACGGGACUUUACUUGGAAAUUUGACUAUCUUCCCACACCAG
CUUAUAAAUUUAAGGACAAACAACUCAAGCACCAUCGUCGUGCCU
UACAUCAAUUGCACUCCUAUGGAUAGCAUGUUACGGCACAACAAU
GUAUCACUUGUUAUCAUACCCAUUUGCCCUUUACGGCCCCCAACC
ACAGGGCAGCAAGUCUUACCGAUCACCAUUUCUAUUGCUCCCAUA
AAAUCAGAGUUUUCAGGGGCUAGGCAGGCCAUUAAACAA

Accordingly, in one embodiment, the immunogenic composition comprises an isolated polynucleotide comprising the nucleotide sequence (RNA) substantially as set out in SEQ ID No: 18, or a variant or fragment thereof.

In one embodiment, the amino acid sequence of the RV-A28 VP0 peptide is provided herein as SEQ ID No: 19, as follows:

[SEQ ID No: 19]
MGAQVSRQNVGTHTTQNAVSNGSSLNYFNINYFKDAASSGASRLD
FSQDPSKFTDPVKDVLTKGIPTLQSPSVEACGYSDRIIQITRGNS
TITSQDIANAVVAYGVWPEYLPSQDASAIDKPTHPDTSSNRFYTL
ESKNWTATSKGWWWKLPDALKNMGIFGENLFYHFLGRAGYTVHVQ
CNASKFHQGTLIVAAIPEHQLAFISKGNVTVGYNYTHPGEQGREI
GTFGPSSTLDRQPSDENWLNCDGTLLGNITVYPHQYINLRSNNSA
TLILPYVNAVPMDSMVRHNNWSIVIIPISKLETEGGTTIVPITLS
ISPMFSEFSGARNNVPQ

Accordingly, preferably the RV-A28 VP0 peptide comprises an amino acid sequence substantially as set out in SEQ ID No: 19, or a variant or fragment thereof.

The VP4 peptide of RV-A28 constitutes amino acid residues 1-69 of SEQ ID No: 19 and the VP2 peptide of RV-A28 constitutes amino acid residues 70-332 of SEQ ID No: 19. Thus, in embodiments in which the immunogenic composition comprises a fragment of RV-A28 VP0, the fragment may comprise or consist of the amino acids forming the VP2 and/or VP4 peptides.

In one embodiment, the RV-A28 VP0 peptide is encoded by the nucleotide sequence (DNA) of SEQ ID No: 20, as follows:

[SEQ ID No: 20]
ATGGGCGCTCAAGTATCTAGGCAGAATGTTGGCACACACACCACA
CAAAATGCTGTATCAAATGGCTCTAGTTTGAATTATTTTAACATC
AACTACTTTAAAGATGCTGCCTCAAGTGGAGCATCACGACTAGAT
TTTTCACAAGATCCTAGTAAATTCACTGATCCAGTTAAAGATGTT
CTTACAAAAGGTATACCAACTCTACAATCACCCAGTGTTGAGGCA
TGTGGTTATTCAGATAGGATCATACAAATAACACGAGGGAATTCA
ACAATTACTTCACAAGATATAGCAAATGCTGTAGTAGCCTATGGA
GTTTGGCCTGAATATCTGCCTAGTCAAGATGCTAGTGCAATAGAC
AAACCAACACATCCTGACACCTCTTCTAACAGATTCTATACATTG
GAAAGCAAAAATTGGACTGCAACCTCAAAAGGATGGTGGTGGAAA
CTTCCAGATGCTCTCAAGAATATGGGGATCTTTGGTGAGAATCTT
TTCTATCATTTCCTTGGTAGAGCCGGGTACACTGTACATGTGCAG
TGTAATGCTAGCAAGTTTCACCAGGGTACCCTAATAGTTGCTGCC
ATTCCAGAACATCAGTTGGCTTTCATTTCCAAGGGTAATGTCACA
GTTGGTTATAATTATACACACCCTGGTGAACAAGGCCGTGAAATA
GGCACCTTTGGACCCAGTAGCACATTGGACAGACAACCTAGTGAT
GAAAATTGGCTTAATTGTGATGGCACACTTTTGGGAAATATAACA
GTGTATCCACACCAATATATCAACCTCAGAAGTAACAATTCAGCA
ACATTAATATTACCATATGTTAACGCCGTACCCATGGACTCTATG
GTTAGGCATAACAATTGGAGTATAGTGATTATACCCATATCTAAA
TTAGAGACTGAGGGTGGGACAACAATTGTACCCATAACTTTATCT
ATTAGCCCAATGTTTTCAGAGTTTTCTGGGGCGAGAAACAATGTC
CCTCAA

Accordingly, preferably the RV-A28 VP0 peptide is encoded by the nucleotide sequence substantially as set out in SEQ ID No: 20, or a variant or fragment thereof. Alternatively, the immunogenic composition comprises an isolated polynucleotide comprising the nucleotide sequence substantially as set out in SEQ ID No: 20, or a variant or fragment thereof.

In one embodiment, the RNA sequence corresponding to the DNA sequence of SEQ ID No: 20 is provided herein as SEQ ID No: 21, as follows:

[SEQ ID No: 21]
AUGGGCGCUCAAGUAUCUAGGCAGAAUGUUGGCACACACACCACA
CAAAAUGCUGUAUCAAAUGGCUCUAGUUUGAAUUAUUUUAACAUC
AACUACUUUAAAGAUGCUGCCUCAAGUGGAGCAUCACGACUAGAU
UUUUCACAAGAUCCUAGUAAAUUCACUGAUCCAGUUAAAGAUGUU
CUUACAAAAGGUAUACCAACUCUACAAUCACCCAGUGUUGAGGCA
UGUGGUUAUUCAGAUAGGAUCAUACAAAUAACACGAGGGAAUUCA
ACAAUUACUUCACAAGAUAUAGCAAAUGCUGUAGUAGCCUAUGGA
GUUUGGCCUGAAUAUCUGCCUAGUCAAGAUGCUAGUGCAAUAGAC
AAACCAACACAUCCUGACACCUCUUCUAACAGAUUCUAUACAUUG
GAAAGCAAAAAUUGGACUGCAACCUCAAAAGGAUGGUGGUGGAAA
CUUCCAGAUGCUCUCAAGAAUAUGGGGAUCUUUGGUGAGAAUCUU
UUCUAUCAUUUCCUUGGUAGAGCCGGGUACACUGUACAUGUGCAG
UGUAAUGCUAGCAAGUUUCACCAGGGUACCCUAAUAGUUGCUGCC
AUUCCAGAACAUCAGUUGGCUUUCAUUUCCAAGGGUAAUGUCACA
GUUGGUUAUAAUUAUACACACCCUGGUGAACAAGGCCGUGAAAUA
GGCACCUUUGGACCCAGUAGCACAUUGGACAGACAACCUAGUGAU
GAAAAUUGGCUUAAUUGUGAUGGCACACUUUUGGGAAAUAUAACA
GUGUAUCCACACCAAUAUAUCAACCUCAGAAGUAACAAUUCAGCA
ACAUUAAUAUUACCAUAUGUUAACGCCGUACCCAUGGACUCUAUG
GUUAGGCAUAACAAUUGGAGUAUAGUGAUUAUACCCAUAUCUAAA
UUAGAGACUGAGGGUGGGACAACAAUUGUACCCAUAACUUUAUCU
AUUAGCCCAAUGUUUUCAGAGUUUUCUGGGGCGAGAAACAAUGUC
CCUCAA

Accordingly, in one embodiment, the immunogenic composition comprises an isolated polynucleotide comprising the nucleotide sequence (RNA) substantially as set out in SEQ ID No: 21, or a variant or fragment thereof.

In one embodiment, the amino acid sequence of the RV-A29 VP0 peptide is provided herein as SEQ ID No: 22, as follows:

[SEQ ID No: 22]
MGAQVSRQNVGTHSTQNSVSNGSSLNYENINYFKDAASSGASKLE
FSQDPSKFTDPVKDVLEKGIPTLQSPTVEACGYSDRIMQITRGDS
TITSQDVANAVIGYGVWPHYLSAEDATAIDKPTQPDTSSNRFYTL
ESKTWNRQSKGWWWKLPDALKDMGIFGENMYYHYLGRSGYTVHVQ
CNASKFHQGTLLVVMIPEHQLASVGTDKVGPGYAFTHPGEAGRQI
GNVSEPTSKHPSDDNWLNFDGTLLGNVLIFPHQFINLRSNNSATI
IVPYVNAVPMDSMLRHNNWSLVIIPISELQAENATNVTVPITVSI
SPMFAEFSGARARPARATTQ

Accordingly, preferably the RV-A29 VP0 peptide comprises an amino acid sequence substantially as set out in SEQ ID No: 22, or a variant or fragment thereof.

The VP4 peptide of RV-A29 constitutes amino acid residues 1-69 of SEQ ID No: 22 and the VP2 peptide of RV-A29 constitutes amino acid residues 70-335 of SEQ ID No: 22. Thus, in embodiments in which the immunogenic composition comprises a fragment of RV-A29 VP0, the fragment may comprise or consist of the amino acids forming the VP2 and/or VP4 peptides.

In one embodiment, the RV-A29 VP0 peptide is encoded by the nucleotide sequence (DNA) of SEQ ID No: 23, as follows:

[SEQ ID No: 23]
ATGGGCGCTCAAGTATCCAGACAAAATGTTGGAACTCATTCTACC
CAAAATTCAGTTTCAAATGGATCGAGTTTAAATTATTTTAACATA
AATTACTTTAAGGATGCTGCATCAAGTGGAGCCTCAAAACTTGAA
TTTTCTCAAGATCCTAGCAAATTCACTGACCCGGTGAAAGATGTT
TTAGAAAAAGGTATTCCAACACTACAATCCCCTACGGTGGAGGCA
TGTGGTTATTCAGATCGGATAATGCAAATTACAAGAGGTGACTCC
ACAATAACATCTCAAGATGTGGCTAATGCAGTTATAGGTTATGGA
GTCTGGCCACACTATCTGAGTGCTGAAGATGCTACTGCTATTGAT
AAACCTACCCAGCCAGACACCTCATCAAATAGATTTTACACCTTG
GAGAGTAAGACATGGAATAGGCAATCCAAAGGATGGTGGTGGAAA
TTGCCAGATGCACTTAAAGATATGGGCATATTTGGAGAAAATATG
TATTATCATTACCTTGGCAGGAGTGGTTATACAGTACATGTCCAA
TGCAATGCTAGTAAGTTTCATCAAGGAACATTACTGGTAGTGATG
ATACCTGAGCACCAACTGGCAAGTGTAGGTACTGATAAAGTCGGA
CCTGGATATGCTTTCACGCATCCCGGTGAGGCAGGTAGACAGATT
GGCAATGTGAGTGAACCGACTTCAAAGCATCCAAGTGATGACAAT
TGGCTCAACTTTGATGGCACCTTACTGGGTAATGTTCTTATATTC
CCACACCAATTTATCAATTTAAGGAGCAATAATTCTGCAACAATA
ATAGITCCTTATGTGAATGCTGTCCCAATGGATTCAATGTTAAGA
CATAATAATTGGAGTTTAGTGATAATTCCAATCAGTGAATTACAA
GCAGAGAATGCCACTAATGTCACGGTCCCTATTACTGTATCAATT
AGTCCAATGTTTGCTGAATTTTCTGGAGCACGTGCAAGGCCAGCA
AGAGCTACTACACAG

Accordingly, preferably the RV-A29 VP0 peptide is encoded by the nucleotide sequence substantially as set out in SEQ ID No: 23, or a variant or fragment thereof. Alternatively, the immunogenic composition comprises an isolated polynucleotide comprising the nucleotide sequence substantially as set out in SEQ ID No: 23, or a variant or fragment thereof.

In one embodiment, the RNA sequence corresponding to the DNA sequence of SEQ ID No: 23 is provided herein as SEQ ID No: 24, as follows:

[SEQ ID No: 24]
AUGGGCGCUCAAGUAUCCAGACAAAAUGUUGGAACUCAUUCUACC
CAAAAUUCAGUUUCAAAUGGAUCGAGUUUAAAUUAUUUUAACAUA
AAUUACUUUAAGGAUGCUGCAUCAAGUGGAGCCUCAAAACUUGAA
UUUUCUCAAGAUCCUAGCAAAUUCACUGACCCGGUGAAAGAUGUU
UUAGAAAAAGGUAUUCCAACACUACAAUCCCCUACGGUGGAGGCA
UGUGGUUAUUCAGAUCGGAUAAUGCAAAUUACAAGAGGUGACUCC
ACAAUAACAUCUCAAGAUGUGGCUAAUGCAGUUAUAGGUUAUGGA
GUCUGGCCACACUAUCUGAGUGCUGAAGAUGCUACUGCUAUUGAU
AAACCUACCCAGCCAGACACCUCAUCAAAUAGAUUUUACACCUUG
GAGAGUAAGACAUGGAAUAGGCAAUCCAAAGGAUGGUGGUGGAAA
UUGCCAGAUGCACUUAAAGAUAUGGGCAUAUUUGGAGAAAAUAUG
UAUUAUCAUUACCUUGGCAGGAGUGGUUAUACAGUACAUGUCCAA
UGCAAUGCUAGUAAGUUUCAUCAAGGAACAUUACUGGUAGUGAUG
AUACCUGAGCACCAACUGGCAAGUGUAGGUACUGAUAAAGUCGGA
CCUGGAUAUGCUUUCACGCAUCCCGGUGAGGCAGGUAGACAGAUU
GGCAAUGUGAGUGAACCGACUUCAAAGCAUCCAAGUGAUGACAAU
UGGCUCAACUUUGAUGGCACCUUACUGGGUAAUGUUCUUAUAUUC
CCACACCAAUUUAUCAAUUUAAGGAGCAAUAAUUCUGCAACAAUA
AUAGUUCCUUAUGUGAAUGCUGUCCCAAUGGAUUCAAUGUUAAGA
CAUAAUAAUUGGAGUUUAGUGAUAAUUCCAAUCAGUGAAUUACAA
GCAGAGAAUGCCACUAAUGUCACGGUCCCUAUUACUGUAUCAAUU
AGUCCAAUGUUUGCUGAAUUUUCUGGAGCACGUGCAAGGCCAGCA
AGAGCUACUACACAG

Accordingly, in one embodiment, the immunogenic composition comprises an isolated polynucleotide comprising the nucleotide sequence (RNA) substantially as set out in SEQ ID No: 24, or a variant or fragment thereof.

In one embodiment, the amino acid sequence of the RV-A89 VP0 peptide is provided herein as SEQ ID No: 25, as follows:

[SEQ ID No: 25]
MGAQVSRQNVGTHSTQNSVSNGSSLNYENINYFKDAASSGASRLD
FSQDPSKFTDPVKDVLEKGIPTLQSPTVEACGYSDRLIQITRGDS
TITSQDIANAVVAYGVWPSYLTPDDATAIDKPTQPDTSSNRFYTL
DSRSWTSASSGWWWKLPDALKNMGIFGENMFYHFLGRSGYTIHVQ
CNSSKFHQGLLIVAAIPEHQLASATSGNVSVGYNHTHPGEQGREV
VPSRTSSDNKRPSDDSWLNFDGTLLGNLPIYPHQYINLRINNSAT
LILPYVNAVPMDSMLRHNNWSLVIIPICPLQVQPGGTQSIPITVS
ISPMFSEFSGPRNKVVFSTTQ

Accordingly, preferably the RV-A89 VP0 peptide comprises an amino acid sequence substantially as set out in SEQ ID No: 25, or a variant or fragment thereof. The VP4 peptide of RV-A89 constitutes amino acid residues 1-69 of SEQ ID No: 25 and the VP2 peptide of RV-A89 constitutes amino acid residues 70-336 of SEQ ID No: 25. Thus, in embodiments in which the immunogenic composition comprises a fragment of RV-A89 VP0, the fragment may comprise or consist of the amino acids forming the VP2 and/or VP4 peptides.

In one embodiment, the RV-A89 VP0 peptide is encoded by the nucleotide sequence (DNA) of SEQ ID No: 26, as follows:

[SEQ ID No: 26]
ATGGGTGCACAAGTATCTAGACAGAATGTTGGGACACACTCCACA
CAGAATTCAGTGAGCAATGGTTCTAGTTTAAATTATTTCAACATC
AATTATTTCAAGGATGCAGCCTCAAGTGGTGCTTCTAGATTAGAT
TTCTCCCAAGATCCTAGTAAATTCACTGACCCTGTTAAAGATGTT
TTAGAAAAAGGTATTCCAACACTCCAGTCACCGACAGTTGAGGCT
TGTGGTTATTCAGACAGGCTAATACAAATAACCCGAGGAGATTCC
ACTATAACGTCTCAGGATATTGCAAATGCAGTTGTCGCTTATGGT
GTTTGGCCATCATACCTAACTCCAGATGATGCGACTGCCATTGAC
AAACCTACACAACCTGATACTTCATCCAATAGATTCTACACTCTG
GATAGTCGCTCTTGGACATCTGCCTCATCTGGTTGGTGGTGGAAA
TTGCCTGATGCCCTTAAGAACATGGGTATCTTTGGTGAAAACATG
TTTTATCACTTTCTAGGGAGATCTGGGTACACAATACATGTACAG
TGTAATTCTAGTAAGTTCCATCAGGGTTTGTTGATAGTTGCTGCT
ATCCCAGAGCATCAATTGGCATCTGCGACAAGTGGAAATGTATCA
GTTGGATATAACCACACCCACCCAGGTGAACAAGGTAGAGAAGTG
GTGCCATCACGGACTTCTAGTGATAACAAAAGACCTAGTGATGAT
AGTTGGTTGAACTTTGATGGGACATTACTTGGTAACCTGCCTATT
TATCCCCACCAATATATTAATTTAAGGACTAATAATTCAGCTACC
CTTATTTTACCTTATGTTAATGCTGTACCAATGGATTCTATGCTT
AGACACAATAATTGGAGTTTAGTTATAATCCCAATATGCCCTCTT
CAAGTTCAACCAGGAGGGACACAATCCATACCTATAACAGTATCA
ATCAGCCCTATGTTTTCAGAATTTTCAGGGCCAAGGAATAAGGTT
GTATTTAGTACCACTCAA

Accordingly, preferably the RV-A89 VP0 peptide is encoded by the nucleotide sequence substantially as set out in SEQ ID No: 26, or a variant or fragment thereof. Alternatively, the immunogenic composition comprises an isolated polynucleotide comprising the nucleotide sequence substantially as set out in SEQ ID No: 26, or a variant or fragment thereof.

In one embodiment, the RNA sequence corresponding to the DNA sequence of SEQ ID No: 26 is provided herein as SEQ ID No: 27, as follows:

[SEQ ID No: 27]
AUGGGUGCACAAGUAUCUAGACAGAAUGUUGGGACACACUCCACA
CAGAAUUCAGUGAGCAAUGGUUCUAGUUUAAAUUAUUUCAACAUC
AAUUAUUUCAAGGAUGCAGCCUCAAGUGGUGCUUCUAGAUUAGAU
UUCUCCCAAGAUCCUAGUAAAUUCACUGACCCUGUUAAAGAUGUU
UUAGAAAAAGGUAUUCCAACACUCCAGUCACCGACAGUUGAGGCU
UGUGGUUAUUCAGACAGGCUAAUACAAAUAACCCGAGGAGAUUCC
ACUAUAACGUCUCAGGAUAUUGCAAAUGCAGUUGUCGCUUAUGGU
GUUUGGCCAUCAUACCUAACUCCAGAUGAUGCGACUGCCAUUGAC
AAACCUACACAACCUGAUACUUCAUCCAAUAGAUUCUACACUCUG
GAUAGUCGCUCUUGGACAUCUGCCUCAUCUGGUUGGUGGUGGAAA
UUGCCUGAUGCCCUUAAGAACAUGGGUAUCUUUGGUGAAAACAUG
UUUUAUCACUUUCUAGGGAGAUCUGGGUACACAAUACAUGUACAG
UGUAAUUCUAGUAAGUUCCAUCAGGGUUUGUUGAUAGUUGCUGCU
AUCCCAGAGCAUCAAUUGGCAUCUGCGACAAGUGGAAAUGUAUCA
GUUGGAUAUAACCACACCCACCCAGGUGAACAAGGUAGAGAAGUG
GUGCCAUCACGGACUUCUAGUGAUAACAAAAGACCUAGUGAUGAU
AGUUGGUUGAACUUUGAUGGGACAUUACUUGGUAACCUGCCUAUU
UAUCCCCACCAAUAUAUUAAUUUAAGGACUAAUAAUUCAGCUACC
CUUAUUUUACCUUAUGUUAAUGCUGUACCAAUGGAUUCUAUGCUU
AGACACAAUAAUUGGAGUUUAGUUAUAAUCCCAAUAUGCCCUCUU
CAAGUUCAACCAGGAGGGACACAAUCCAUACCUAUAACAGUAUCA
AUCAGCCCUAUGUUUUCAGAAUUUUCAGGGCCAAGGAAUAAGGUU
GUAUUUAGUACCACUCAA

Accordingly, in one embodiment, the immunogenic composition comprises an isolated polynucleotide comprising the nucleotide sequence (RNA) substantially as set out in SEQ ID No: 27, or a variant or fragment thereof.

The term “immunogenic composition” as used throughout, refers to a composition of matter (intended to be administered to a subject) that comprises at least one antigen or induces the expression of at least one antigen of a rhinovirus (in the case of nucleic acid immunisation), which has the capability to elicit an immunological response in the subject to which it is administered. Such an immune response can be a cellular and/or antibody-mediated immune response directed at least against the antigen of the composition.

The immunogenic composition described herein provides an effective means of vaccinating a subject against RV infection. Accordingly, in a preferred embodiment, the immunogenic composition is a vaccine.

The immunogenic composition may comprise a suitable adjuvant. In a preferred embodiment, the adjuvant is a Th1 adjuvant. Examples of Th1 adjuvants promoting a Th1 immune response include TLR-9 agonists such as CpG oligonucleotides, or TLR-4 agonists. Alternatively in another embodiment, the adjuvant is Incomplete Freund's Adjuvant (IFA).

Further examples of adjuvants may include an aluminium salt, a synthetic form of DNA, a carbohydrate, a tablet binder, an ion exchange resin, a preservative, a polymer, an emulsion and/or a lipid. Examples of adjuvants may include monosodium glutamate, sucrose, dextrose, aluminum bovine, human serum albumin, cytosine phosphoguanine, potassium phosphate, plasdone C, anhydrous lactose, cellulose, polacrilin potassium, glycerine, asparagine, citric acid, potassium phosphate magnesium sulfate, iron ammonium citrate, 2-phenoxyethanol, aluminium, beta-propiolactone, bovine extract, DOPC, EDTA, formaldehyde, thimerosal, phenol, potassium aluminum sulfate, potassium glutamate, sodium borate, sodium metabisulphite, urea, PLGA, PVA, PLA, PVP, cyclodextrin-based stabilisers, oil in water emulsion adjuvants and/or lipid-based adjuvants.

The immunogenic composition according to the first aspect is particularly suitable for therapy or prophylaxis (i.e. vaccination) against rhinovirus infections.

Hence, in a second aspect of the invention, there is provided the immunogenic composition according to the first aspect, for use in therapy or prophylaxis.

In a third aspect, there is provided the immunogenic composition according to the first aspect, for use in eliciting an immune response.

In a fourth aspect, there is provided a method of eliciting an immune response in a subject, the method comprising administering, to a subject in need thereof, a therapeutically effective amount of an immunogenic composition according to the first aspect.

It will be appreciated that the use and method of the invention comprises vaccination.

In a preferred embodiment, the immunogenic composition for use according to the third aspect, or the method according to the fourth aspect, elicits an immune response against a rhinovirus infection.

Preferably, the immunogenic composition for use according to the third aspect, or the method according to the fourth aspect, elicits an immune response against at least one, at least two, or at least three species of rhinoviruses, more particularly, against RV-A, RV-B, and/or RV-C.

Advantageously, the immune response that is induced by the immunogenic composition according to the first aspect, is a specific cell-mediated immune response not only directed to the homologous strain(s) of rhinovirus from which the immunogenic composition is derived but also to other (heterologous) strains of rhinoviruses of the same species of rhinoviruses, which can extend to strains of rhinoviruses of another group of rhinoviruses.

Preferably, therefore, the immunogenic composition for use according to the third aspect, or the method according to the fourth aspect, elicits an immune response against at least one strain of RV-A, selected from a group consisting of: RV-1A, RV-A1, RV-A10, RV-A100, RV-A101, RV-A103, RV-A106, RV-A107, RV-A108, RV-A11, RV-A12, RV-A13, RV-A15, RV-A16, RV-A18, RV-A19, RV-A1B, RV-A2, RV-A20, RV-A21, RV-A22, RV-A23, RV-A24, RV-A25, RV-A28, RV-A29, RV-A30, RV-A31, RV-A32, RV-A33, RV-A34, RV-A36, RV-A38, RV-A39, RV-A40, RV-A41, RV-A43, RV-A44, RV-A45, RV-A46, RV-A47, RV-A49, RV-A50, RV-A51, RV-A53, RV-A54, RV-A55, RV-A56, RV-A57, RV-A58, RV-A59, RV-A60, RV-A61, RV-A62, RV-A63, RV-A64, RV-A65, RV-A66, RV-A67, RV-A68, RV-A7, RV-A71, RV-A73, RV-A74, RV-A75, RV-A76, RV-A77, RV-A78, RV-A8, RV-A80, RV-A81, RV-A82, RV-A85, RV-A88, RV-A89, RV-A9, RV-A90, RV-A94, RV-A95, RV-A96, and RV-A98. In a preferred embodiment, the immunogenic composition for use according to the third aspect, or the method according to the fourth aspect, elicits an immune response against all strains of RV-A.

Preferably, the immunogenic composition for use according to the third aspect, or the method according to the fourth aspect, elicits an immune response against at least one strain of RV-B, selected from a group consisting of: RV-B103, RV-B102, RV-B101, RV-B100, RV-B27, RV-B26, RV-B4, RV-B97, RV-B35, RV-B84, RV-B93, RV-B92, RV-B91, RV-B17, RV-B5, RV-B42, RV-B6, RV-B37, RV-B48, RV-B69, RV-B52, RV-B72, RV-B3, RV-B14, RV-B99, RV-B86, RV-B83, RV-B79, and RV-B70. In a preferred embodiment, the immunogenic composition for use according to the third aspect, or the method according to the fourth aspect, elicits an immune response against all strains of RV-B.

Preferably, the immunogenic composition for use according to the third aspect, or the method according to the fourth aspect, elicits an immune response against at least one strain of RV-C, selected from a group consisting of: RV-C58, RV-C50, RV-C48, RV-C46, RV-C13, RV-C51, RV-C55, RV-C56, RV-C45, RV-C44, RV-C41, RV-C4, RV-C38, RV-C34, RV-C33, RV-C31, RV-C30, RV-C29, RV-C27, RV-C26, RV-C24, RV-C23, RV-C21, RV-C16, RV-C14, RV-C47, RV-C53, RV-C54, RV-C22, RV-C37, RV-C49, RV-C25, RV-C18, RV-C12, RV-C20, RV-C19, RV-C28, RV-C36, RV-C39, RV-C15, RV-C5, RV-C11, RV-C9, RV-C10, RV-C1, RV-C43, RV-C42, RV-C40, RV-C32, RV-C17, RV-C8, RV-C7, RV-C6, RV-C3, RV-C2, and RV-C35. In a preferred embodiment, the immunogenic composition for use according to the third aspect, or the method according to the fourth aspect, elicits an immune response against all strains of RV-C.

Furthermore, it is well-known that rhinoviruses are the major cause of common colds, virus-induced wheezing illnesses in early childhood and acute attacks (exacerbations) of lung diseases such as asthma, bronchiectasis, chronic obstructive pulmonary disease (COPD), cystic fibrosis and chronic fibrosing lung diseases.

Accordingly, in a preferred embodiment, the immunogenic composition for use according to the third aspect, or the method according to the fourth aspect, elicits an immune response against common colds, virus-induced wheezing illnesses, acute attacks of lung diseases such as asthma, exacerbations of asthma, bronchiectasis, chronic obstructive pulmonary disease (COPD), cystic fibrosis and/or chronic fibrosing lung disease.

As used herein, the expression “eliciting an immune response” may involve inducing a specific cell-mediated immune response. This means the generation of a specific T lymphocyte response following the administration of an immunogenic composition in a subject. The two main cellular effectors of the specific T lymphocyte response are the helper T-cells and the cytotoxic T lymphocytes (CTLs).

CD4+ “helper” T-cells or helper T-cells, are immune response mediators, and play an important role in establishing and maximizing the capabilities of the adaptive immune response. These cells can have to some extent a direct cytotoxic activity, but, in essence “manage” the immune response, by directing other cells involved in the protection of organisms against pathogens. The activation of a naive helper T-cell causes it to release cytokines, which influences the activity of many cell types such as B lymphocytes, CTLs, and APCs (Antigen Presenting Cells) that activated it. Helper T-cells require a much milder activation stimulus than cytotoxic T-cells. Helper T-cells can provide extra signals that “help” activate cytotoxic cells. Two types of effector CD4+ helper T cell responses can be induced by a professional APC, designated Th1 and Th2. The measure of cytokines associated with Th1 or Th2 responses will give a measure of successful immunisation. This can be achieved by specific ELISA or ELISPOT designed for measurement of Th1-cytokines such as IFN-γ, IL-2, and others, or Th2-cytokines such as IL-4, IL-5, IL-13 among others.

As used herein, the expression “helper T-cell-mediated immune response” refers to an immune response wherein CD4⁺ T-cells or helper T-cells are activated and secrete lymphokines to stimulate both cell-mediated and antibody-mediated branches of the immune system. As known from the skilled person, helper T-cell activation promotes lymphokine secretion, immunoglobulin isotype switching, affinity maturation of the antibody response, macrophage activation and/or enhanced activity of natural killer and cytotoxic T-cells. Lymphokines are proteins secreted by lymphocytes that affect their own activity and/or the activity of other cells. Lymphokines include, but are not limited to, interleukins and cytokines, e.g., IL-2, IL-4, IL-5, IL-6, IL-10, IL-12, or IFN-γ.

It is well known to the skilled person that helper T-cells differentiate into two major subtypes of cells known as Th1 and Th2 cells (also known as Type 1 and Type 2 helper T cells, respectively).

Additionally, it is well known to the skilled person that Th1 cells mainly secrete IL-2 and IFN-γ. They promote cellular immune response by maximizing the killing efficacy of macrophages and the proliferation of cytotoxic CD8⁺ T-cells. Additionally, the type 1 cytokine IFN-γ increases the production of IL-12 by dendritic cells and macrophages, and, via positive feedback, IL-12 stimulates the production of IFN-γ in helper T-cells, thereby promoting the Th1 profile. IFN-γ also inhibits the production of cytokines such as IL-4, an important cytokine associated with the Type 2 response, and thus it also acts to preserve its own response.

On the contrary, Th2 cells mainly secrete IL-4, IL-5 and IL-13, and promote humoral immune response by stimulating B cells into proliferation, inducing B-cell antibody class switching. The Type 2 response further promotes its own profile using two different cytokines. IL-4 acts on helper T-cells to promote the production of Th2 cytokines (including itself), while IL-10 inhibits a variety of cytokines including IL-2 and IFN-γ in helper T-cells and IL-12 in dendritic cells and macrophages.

Preferably, the cell-mediated immune response induced by the immunogenic composition of the invention is primarily a Th1 cell-mediated immune response. In a preferred embodiment, the immunogenic composition for use according to the third aspect, or the method according to the fourth aspect, induces an immune response through the secretion of IFN-γ.

A critical component of protective cellular immunity elicited by natural infection with respiratory viruses is the formation of tissue resident memory T cells (T_{RM}) in the airway mucosa and lungs. These T_{RM} cells rapidly expand and clear subsequent infections. Accordingly, in another preferred embodiment, the immunogenic composition for use according to the third aspect, or the method according to the fourth aspect, induces an immune response through the formation of tissue resident memory T cells (T_{RM}).

In another preferred embodiment, the immunogenic composition for use according to the third aspect, or the method according to the fourth aspect, induces an immune response through the production of RV VP0-specific IgG antibodies.

Alternatively, in another embodiment, immunogenic composition for use according to the third aspect, or the method according to the fourth aspect, induces a cytotoxic T cell immune response.

Cytotoxic T cells (also known as Tc, killer T cell, or cytotoxic T-lymphocyte (CTL)), which express generally the CD8 marker, are a sub-group of T cells and may also be involved in the T cell-mediated immune response. They induce the death of cells that are infected with viruses (and other pathogens). These CTLs directly attack other cells carrying certain foreign or abnormal molecules on their surface. The ability of such cellular cytotoxicity can be detected using in vitro cytolytic assays (chromium release assay). Thus, induction of a specific cellular immunity can be demonstrated by the presence of such cytotoxic T cells, when antigen-loaded target cells are lysed by specific CTLs that are generated in vivo following vaccination or infection.

Similarly to helper T-cells, CD8⁺ T-cells include distinct subsets, which were termed, analogously to the Th1/Th2 terminology, Tc1 and Tc2.

The Tc1 immune response involves specific IFN-γ-producing CD8⁺ T-cells which are activated, proliferate and produce IFN-γ upon specific antigen stimulation. The level of IFN-γ-producing CD8⁺ T-cells can be measured by ELISPOT and by flow cytometry measurement of intracellular IFN-γ in these cells.

Naive cytotoxic T cells are activated when their T-cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed” effector cells. Activated CTL will then travel throughout the body in search of cells bearing that unique MHC Class I+peptide. This could be used to identify such CTLs in vitro by using peptide-MHC Class I tetramers in flow cytometric assays.

When exposed to these infected cells, effector CTL release perforin and granulysin, cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cellular immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-γ, quantitative measurement of IFN-γ-producing CD8 cells can be achieved by ELISPOT and by flow cytometric measurement of intracellular IFN-γ in these cells.

It will be appreciated that the immunogenic composition according to the invention, may be used in a medicament, which may be used as a monotherapy (i.e. use of the immunogenic composition alone), for vaccination against an RV infection. Alternatively, the immunogenic composition according to the invention may be used as an adjunct to, or in combination with, known therapies for treating, ameliorating, or preventing an RV infection.

The immunogenic composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, lipid nanoparticles (e.g. with peptide, DNA or RNA on the surface or encapsulated) or any other suitable form that may be administered to a person or animal in need of vaccination. The lipid nanoparticle may comprise one or more components selected from a group consisting of: a cationic lipid (which is preferably ionisable); phosphatidylcholine; cholesterol; and polyethylene glycol (PEG)-lipid. It will be appreciated that the vehicle of medicaments according to the invention should be one which is well-tolerated by the subject to whom it is given.

Medicaments comprising the immunogenic composition of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin.

The immunogenic composition of the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the immunogenic composition is required and which would normally require frequent administration (e.g. at least daily injection).

In a preferred embodiment, however, medicaments according to the invention may be administered to a subject by injection into the blood stream, muscle, skin or directly into a site requiring treatment. Injections may be intravenous (bolus or infusion) or subcutaneous (bolus or infusion), or intradermal (bolus or infusion), or intramuscular (bolus or infusion).

It will be appreciated that the amount of immunogenic composition that is required is determined by its biological activity and bioavailability, which in turn depends on the mode of administration, the physiochemical properties of the immunogenic composition and whether it is being used as a monotherapy or in a combined therapy. The frequency of administration will also be influenced by the half-life of the active agent within the subject being treated. Optimal dosages to be administered may be determined by those skilled in the art, and will vary with the particular the immunogenic composition in use, the strength of the composition, the mode of administration, and the type and advancement of the viral infection. Additional factors depending on the particular subject being treated will result in a need to adjust dosages, including subject age, weight, gender, diet, and time of administration.

Generally, a daily dose of between 0.001 μg/kg of body weight and 100 mg/kg of body weight of the immunogenic composition of the invention may be used for the immunisation, depending upon the agent used. More preferably, the daily dose of agent is between 1 μg/kg of body weight and 100 mg/kg of body weight, more preferably between 10 μg/kg and 10 mg/kg body weight, and most preferably between approximately 100 μg/kg and 10 mg/kg body weight.

Daily doses may be given as a single administration (e.g. a single daily injection or inhalation of a nasal spray). Alternatively, the immunogenic composition may require administration twice or more times during a day. As an example, the immunogenic composition may be administered as an initial primer and a subsequent boost(s), or two boosts administered at between a week or monthly intervals. Preferably, the immunogenic composition may be administered as an initial primer and a subsequent boost, administered between two to six weeks apart. Preferably, subsequent boosts may then be administered yearly to susceptible patients, such as those with weakened immune systems. Known procedures, such as those conventionally employed by the pharmaceutical industry (e.g. in vivo experimentation, clinical trials, etc.), may be used to form specific formulations of the immunogenic composition according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the immunogenic composition is any amount which, when administered to a subject, is the amount of the aforementioned that is needed to ameliorate, prevent or treat any given disease, preferably prophylactically.

For example, the immunogenic composition of the invention may be used from about 0.001 μg to about 1 mg, and preferably from about 0.001 μg to about 500 μg. It is preferred that the amount of the immunogenic composition is an amount from about 0.01 μg to about 250 μg, and most preferably from about 0.1 μg to about 100 μg. Preferably, the immunogenic composition according to the invention is administered at a dose of 1-50 μg.

The immunogenic compositions of the invention may further comprise a pharmaceutically acceptable vehicle. A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. immunogenic composition according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, tale, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The immunogenic composition according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, subcutaneous, intradermal, intrathecal, epidural, intraperitoneal, intravenous and particularly intramuscular injection. The nucleic acid sequence, or expression cassette of the invention may be prepared as a sterile solid composition that may be dissolved or suspended at the time of administration using sterile water, saline, or other appropriate sterile injectable medium.

The immunogenic composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The immunogenic composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-27 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 27 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

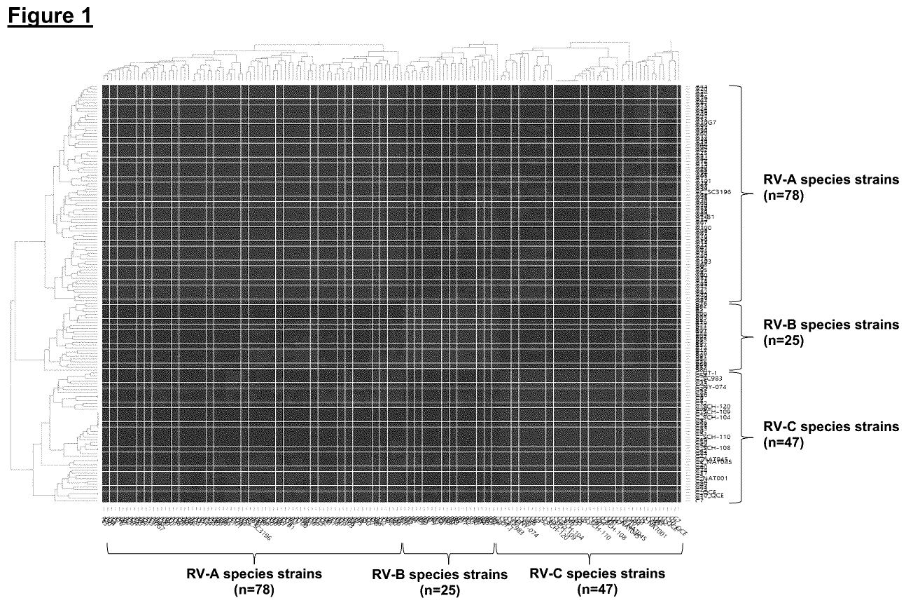

FIG. 1 shows a heatmap visualising pairwise identity scores between 150 rhinovirus strain VP0 protein sequences calculated using the MUSCLE algorithm after hierarchical clustering. Pairwise identify scores above (>59.9%, blue) or below (<59.9%, red) the thresholds predicted to be required for cross-immunization are shown.

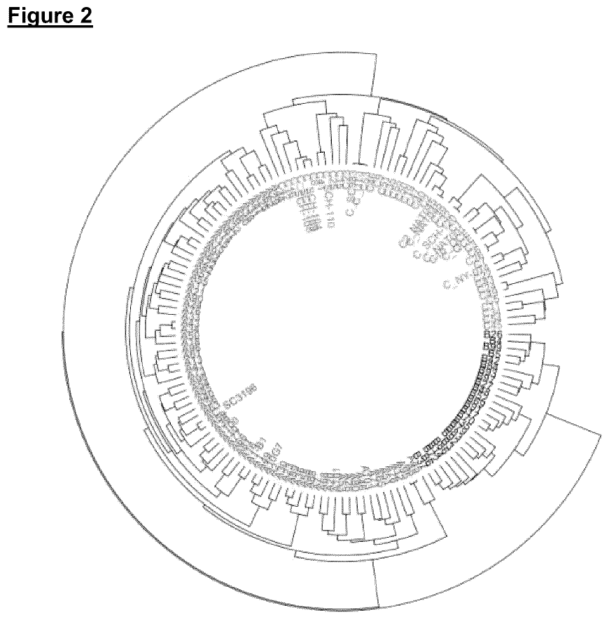

FIG. 2 shows circular tree clustering 150 rhinovirus strain VP0 protein sequences into 3 clusters. Of the sequences analysed, cluster 1 (red) contains all RV-A species VP0 sequences, cluster 2 (black) contains all RV-B species VP0 sequences and cluster 3 (green) contains all RV-C species VP0 sequences.

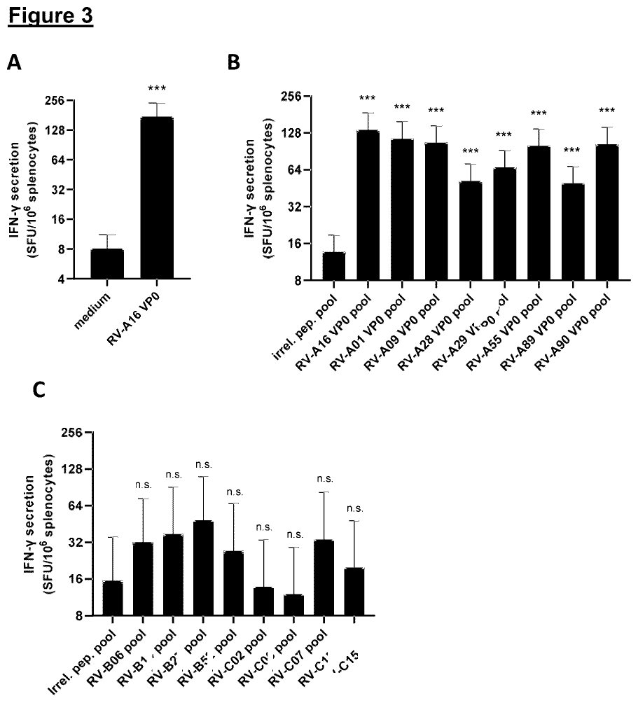

FIG. 3 is a set of histograms representing the number of IFN-γ producing splenocytes from mice immunised with RV-A16 VP0 protein after stimulation with RV-A16 VP0 (A) or the indicated RV-A (B) or RV-B/C (C) species peptide pools. Mice were sacrificed 35-49 days after first immunisation, splenocytes harvested, stimulated as indicated and IFN-γ producing splenocytes (/10⁶ splenocytes) enumerated by ELISPOT. (A) & (B) n=24 mice, data pooled from 3 independent experiments; (C) n=5 mice. Data is presented as geomean±95% confidence intervals. Significant differences were compared to irrelevant peptide using a linear mixed effects model ***: p<0.0001; n.s.: non-significant.

FIG. 4 is a set of histograms representing the number of IFN-γ producing splenocytes from mice immunised with RV-B06 VP0 protein after stimulation with RV-B06 VP0 (A) or the indicated RV-B (B) or RV-A/C species peptide pools (C). Mice were sacrificed 35-49 days after first immunisation, splenocytes harvested, stimulated as indicated and IFN-γ producing splenocytes (/10⁶ splenocytes) enumerated by ELISPOT. (A) & (B) n=24 mice, data pooled from 3 independent experiments; (C) n=7 mice. Data is presented as geomean±95% confidence intervals. Significant differences were compared to irrelevant peptide using a linear mixed effects model. ***: p<0.0001; **: p<0.001; *p<0.01; n.s.: non-significant.

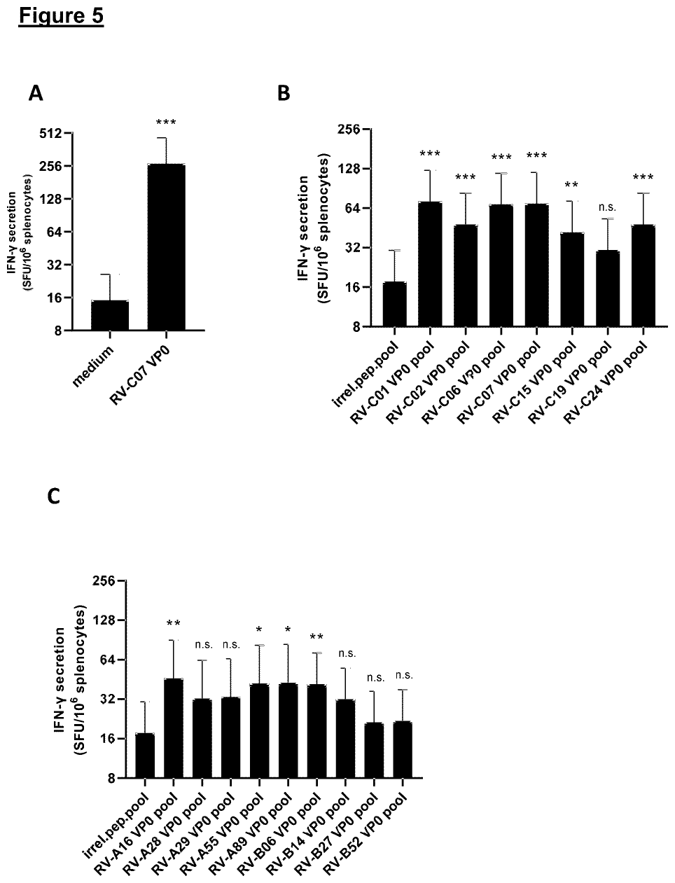

FIG. 5 is a set of histograms representing the number of IFN-γ producing splenocytes from mice immunised with RV-C07 VP0 protein after stimulation with RV-C07 VP0 (A) or the indicated RV-C(B) or RV-A/B species (C) peptide pools. Mice were sacrificed 35-49 days after first immunisation, splenocytes harvested, stimulated as indicated and IFN-γ producing splenocytes (/10⁶ splenocytes) enumerated by ELISPOT. n=7 mice; data pooled from 2 independent experiments. Data is presented as geomean±95% confidence intervals. Significant differences were compared to irrelevant peptide using a linear mixed effects model. ***: p<0.0001; **: p<0.01; *p<0.05; n.s.: non-significant.

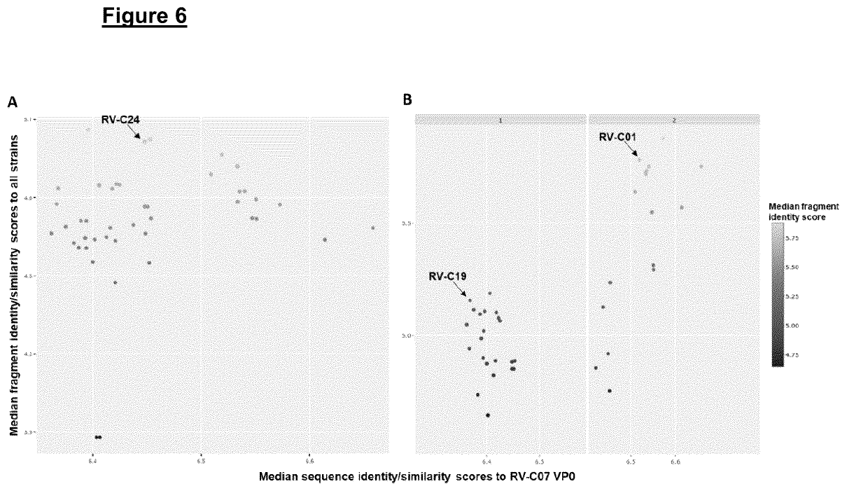

FIG. 6 is a set of dot plot showing the median fragment identity/similarity scores for each RV-C strain VP0 sequence across all other RV-C VP0 strains plotted against the median sequence identity/similarity score for each strain against RV-C07 alone. (A) All C strains plotted as a single cluster. (B) C strains split into two clusters.

FIG. 7 is a set of histograms representing the number of IFN-γ producing splenocytes from mice immunised with RV-C01VP0 protein after stimulation with RV-C01 VP0 (A) or the indicated RV-C(B) or RV-A/B species (C) peptide pools. Mice were sacrificed 35-49 days after first immunisation, splenocytes harvested, stimulated as indicated and IFN-γ producing splenocytes (/10⁶ splenocytes) enumerated by ELISPOT. n=7 mice; data pooled from 2 independent experiments. Data is presented as geomean f₉₅% confidence intervals. Significant differences were compared to irrelevant peptide using a linear mixed effects model. ***: p<0.0001; **: p<0.01; *p<0.05; n.s.: non-significant.

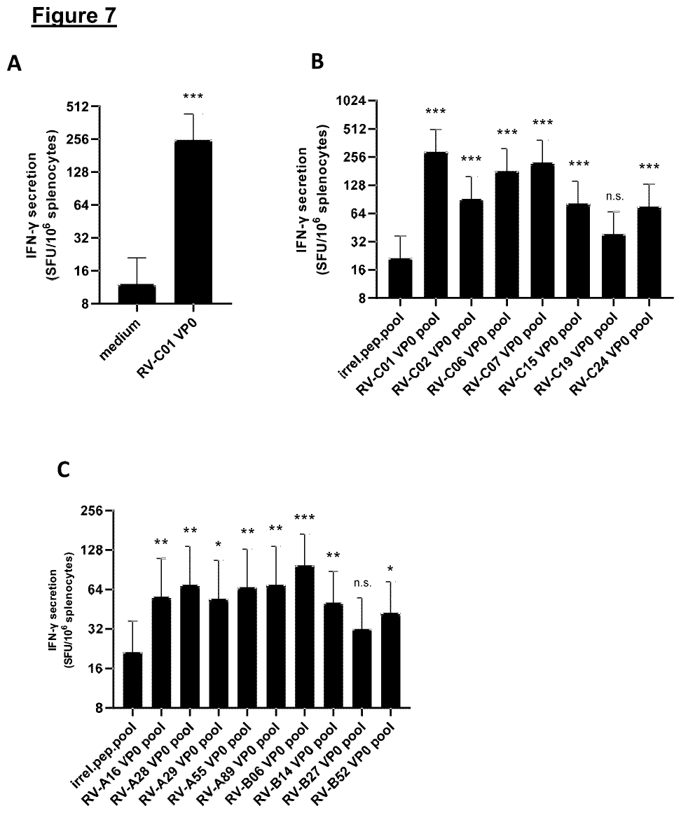

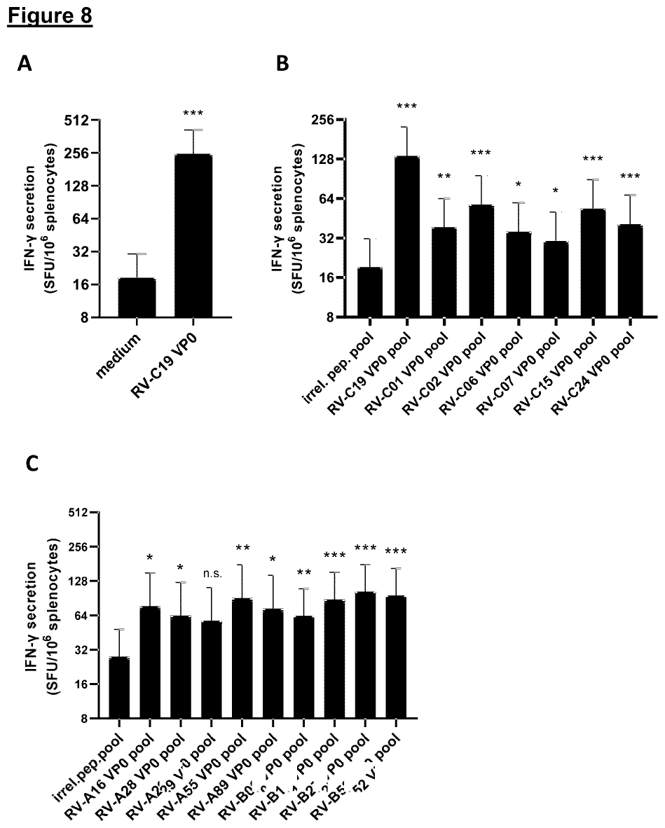

FIG. 8 is a set of histograms representing the number of IFN-γ producing splenocytes from mice immunised with RV-C19 VP0 protein after stimulation with RV-19 VP0 (A) or the indicated RV-C(B) or RV-A/B (C) species peptide pools. Mice were sacrificed 35-49 days after first immunisation, splenocytes harvested, stimulated as indicated and IFN-γ producing splenocytes (/10⁶ splenocytes) enumerated by ELISPOT. N=12 mice; data pooled from 2 independent experiments. Data is presented as geomean±95% confidence intervals. Significant differences were compared to irrelevant peptide using a linear mixed effects model. ***: p<0.0001; **: p<0.001; *p<0.01; n.s.: non-significant.

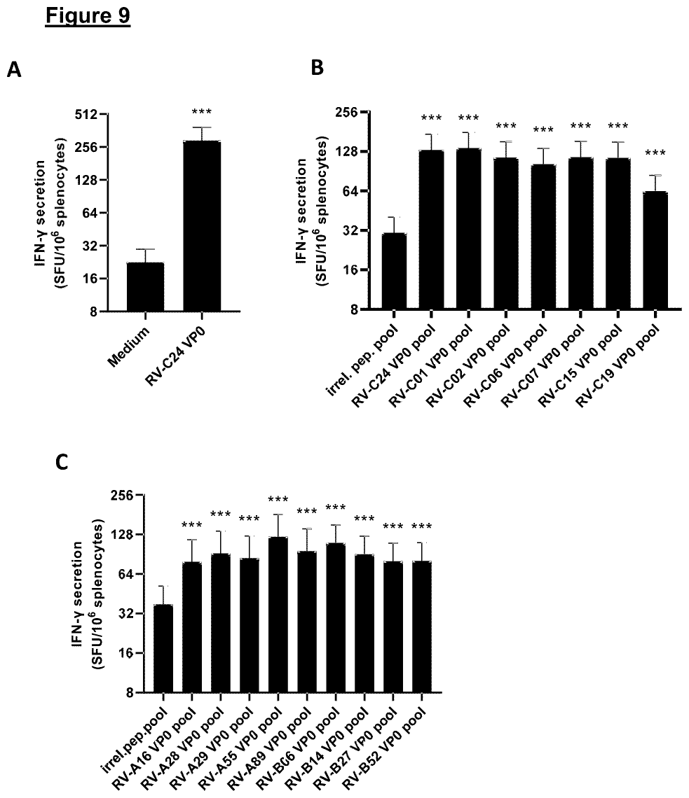

FIG. 9 is a set of histograms representing the number of IFN-γ producing splenocytes from mice immunised with RV-C24 VP0 protein after stimulation with RV-C24 VP0 (A) or the indicated RV-C(B) or RV-A/B (C) species peptide pools. Mice were sacrificed 35-49 days after first immunisation, splenocytes harvested, stimulated as indicated and IFN-γ producing splenocytes (/10⁶ splenocytes) enumerated by ELISPOT. (A) & (B) n=26 mice, data pooled from 2 independent experiments; (C) n=7 mice. Data is presented as geomean±95% confidence intervals. Significant differences were compared to irrelevant peptide using a linear mixed effects model. ***: p<0.0001.

FIG. 10 is a line graph representing the titre of anti-RV VP0 antibodies from mice immunised twice with either RV-B06, RV-C01, RV-C07, RV-C19 or RV-C24 VP0 proteins or controls (PBS or vaccine adjuvant alone). Serum was collected 42 days after first immunisation and the titres of immunogen-specific IgG2a were quantified by ELISA. Each curve represents the titre of the anti-RV VP0 IgG2a specific to VP0 protein the mice were immunised with (n=7 mice/immunogen, mean+/−SEM). Sera from control immunised mice (adjuvant alone or PBS, n=7 per group) were pooled and the respective pooled sera tested in each VP0-specific ELISA (mean+/−SEM of responses across VP0 antigens).

FIG. 11 is a set of histograms that show the results from ELISPOT experiments to analyse the cellular immunity evoked in cynomolgus monkeys by immunisation with rRV-C24 VP0 protein (FIG. 11B) and adjuvant only (control; FIG. 11A) against other strains of RV. Bars represent mean activity/2×10⁵ PBMCs from 3 technical replicates+/−SEM.

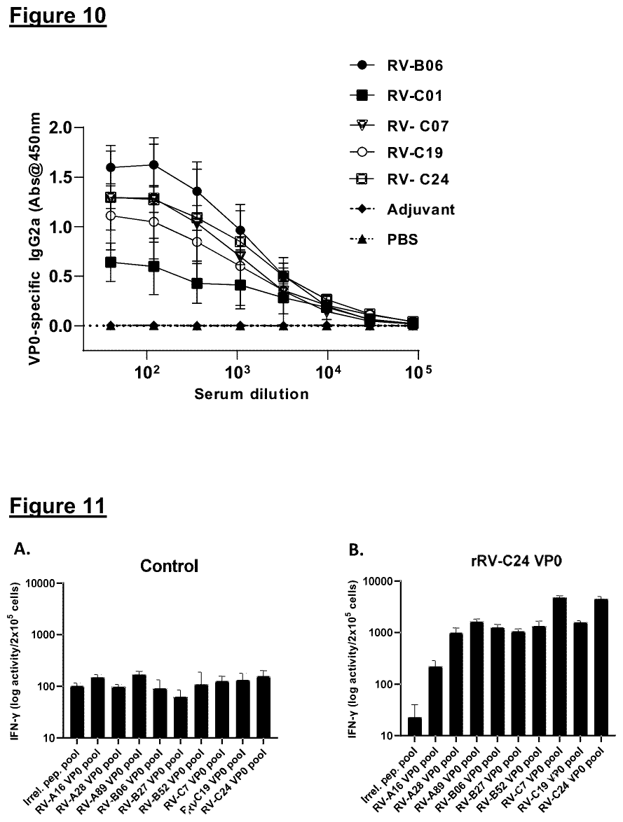

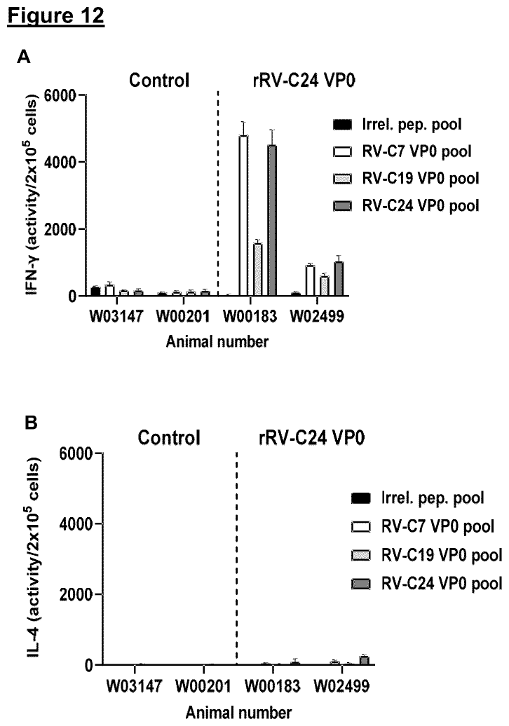

FIG. 12 shows IFN-γ secretion (FIG. 12A) and IL-4 secretion (FIG. 12B) in cynomolgus monkeys immunised with rRV-C24 VP0 protein or adjuvant only (control), in response to stimulation with peptide pools from C species of RV. Bars represent mean activity/2×10⁵ PBMCs from 3 technical replicates+/−SEM.

FIG. 13 is a histogram showing the concentration of RV-C24 VP0-binding IgG, as quantified by ELISA, in the plasma of cynomolgus monkeys at different timepoints after immunization with rRV-C24 VP0 protein. Bars represent mean IgG concentration, and dots represent individual animal responses. Arrows indicate when animals were immunized.

FIG. 14 is a Western blot of HEK293 lysates made at different timepoints after transfection with RV VP0 mRNA. The marker lane is designed M, with bands at 38 and 28 kDa labelled. The predicted molecular weight of RV VP0 protein is approximately 36 kDa. FIG. 14A is a Western blot of lysates from HEK293 cells transfected with RV-A16 VP0 mRNA made at 24, 48, 72 or 96 hours after transfection. A positive control of recombinant RV-A16 VP0 protein was loaded onto the gel (labelled rRV-A16 VP0). Detection of RV-A16 VP0 protein was performed using a monoclonal anti-RV-A16 VP0 antibody. FIG. 14B is a Western blot of lysates from HEK293 cells transfected with RV-B06 or RV-C24 VP0 mRNA made at 24, 48, 72 or 96 hours after transfection. Detection of RV-B06 and C24 VP0 proteins was performed using antisera from mice immunized with either recombinant RV-B06 or recombinant RV-C24 VP0 protein, respectively.

FIG. 15 is a histogram that shows the results from ELISPOT experiments to analyse the cellular immunity in mice evoked by immunisation with RV-C24 VP0 mRNA against other strains of RV. Bars represent mean activity/5×10⁵ splenocytes+/−SEM from 6 animals. *p<0.05, ANOVA.

FIG. 16 is a histogram showing the concentration of RV-C24 VP0-binding IgG, as quantified by ELISA, in the serum of mice at different timepoints after immunization with RV-C24 VP0 mRNA. Bars indicate mean IgG concentration+/−SEM. Arrows indicate when animals were immunized.

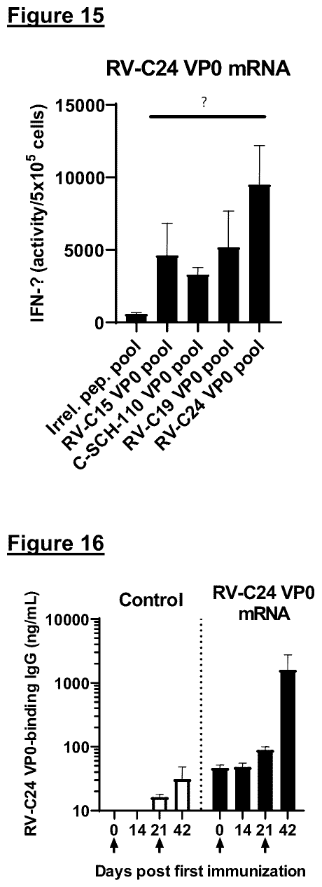

FIG. 17 is a histogram that show the results from ELISPOT experiments to analyse the cellular immunity evoked by immunization of cynomolgus monkeys with an mRNA concatemer consisting of RV-C24/A16/B06 VP0 mRNAs in sequence separated by a 2A peptide-encoding sequence. IFN-γ (FIG. 17A) or IL-4 (FIG. 17B) secretion was quantified by ELISPOT. Bars represent mean activity/2×10⁵ PBMCs from 3 technical replicates+/−SEM.

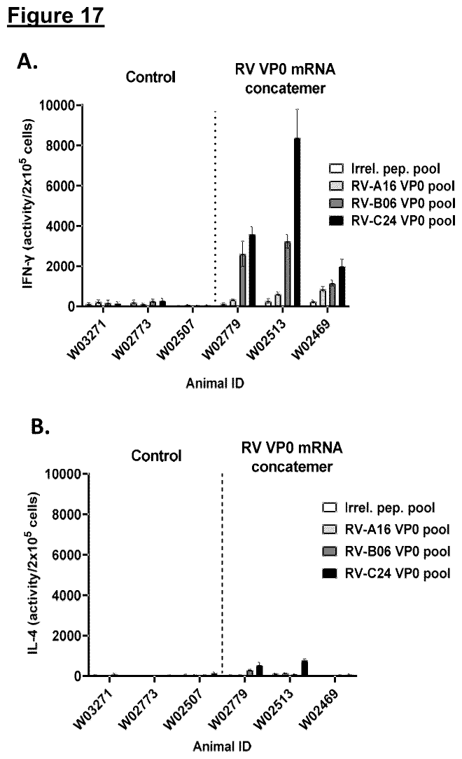

FIG. 18 is a histogram showing the concentration of RV-A16 (FIG. 18A), RV-B06 (FIG. 18B) and RV-C24 (FIG. 18C) VP0-binding IgG, as quantified by ELISA, in the plasma at different timepoints after immunization of a cynomolgus monkey with an mRNA concatemer consisting of RV-C24/A16/B06 VP0 mRNAs in sequence separated by a 2A peptide-encoding sequence. Arrows indicate when the animal was immunized. Bars indicate the IgG concentration detected in a single animal. Each graph was generated using data from the same animal.

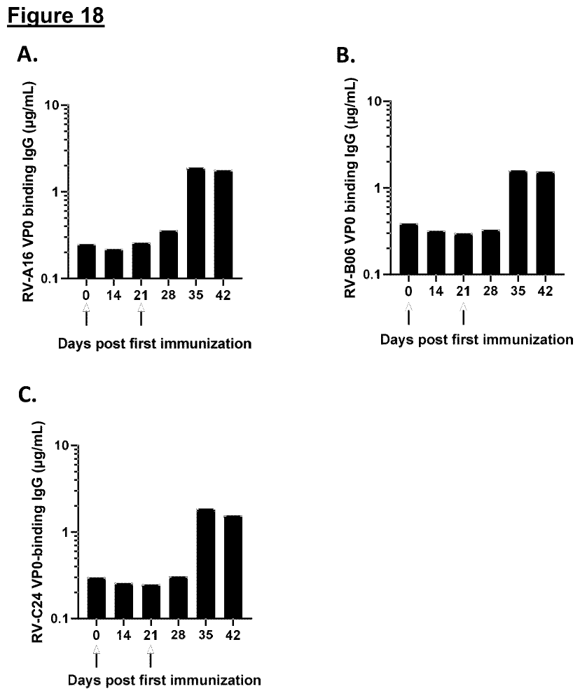

FIG. 19 is a histogram that shows the concentration of RV-A01 RNA in the lungs of control and RV-A16 VP0 mRNA immunized mice, as determined by RT-qPCR, 1, 6 and 14 days after infection with RV-A01. Bars represent mean RV-A01 RNA+/−SEM from 8 animals. *p<0.05, ANOVA.

FIG. 20 is a histogram that shows the results from ELISPOT experiments to analyse how cellular immunity in mice formed by immunisation with RV-A16 VP0 mRNA responds to subsequent infection with heterotypic RV-A01 in splenocytes harvested 14 days after infection with RV-A1. Bars represent mean spot forming units/1×10⁶ splenocytes+/−SEM from 8 animals. *p<0.05, ***p<0.001, ANOVA.

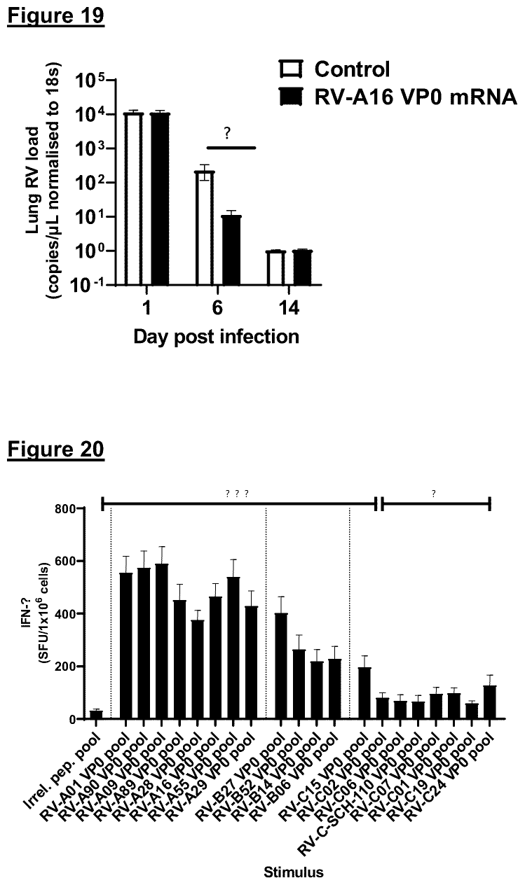

FIG. 21 is a histogram that shows the results from ELISPOT experiments to analyse cellular immunity from control mice whose spleens were also harvested 14 days after infection with RV-A01. Bars represent mean spot forming units/1×10⁶ splenocytes+/−SEM from 8 animals.

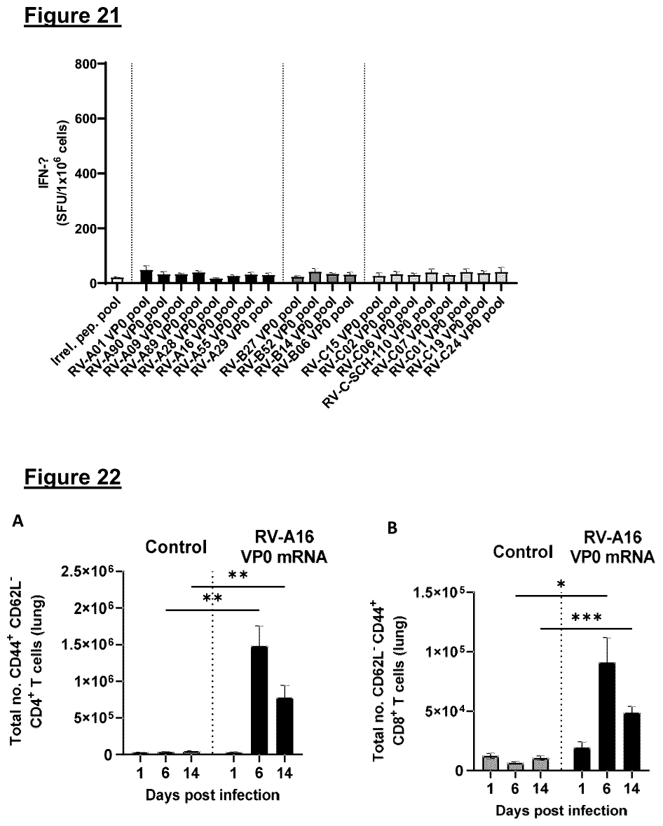

FIG. 22 is a histogram that shows the mean numbers of CD62⁻ CD44⁺ effector CD4⁺ (FIG. 22A) and CD8⁺ (FIG. 22B) T cells, respectively, in the lungs of RV-A16 VP0 mRNA immunized and control mice after infection with RV-A1. Bars represent mean cell numbers+/−SEM from 8 animals. **p<0.01, ***p<0.001, ANOVA.

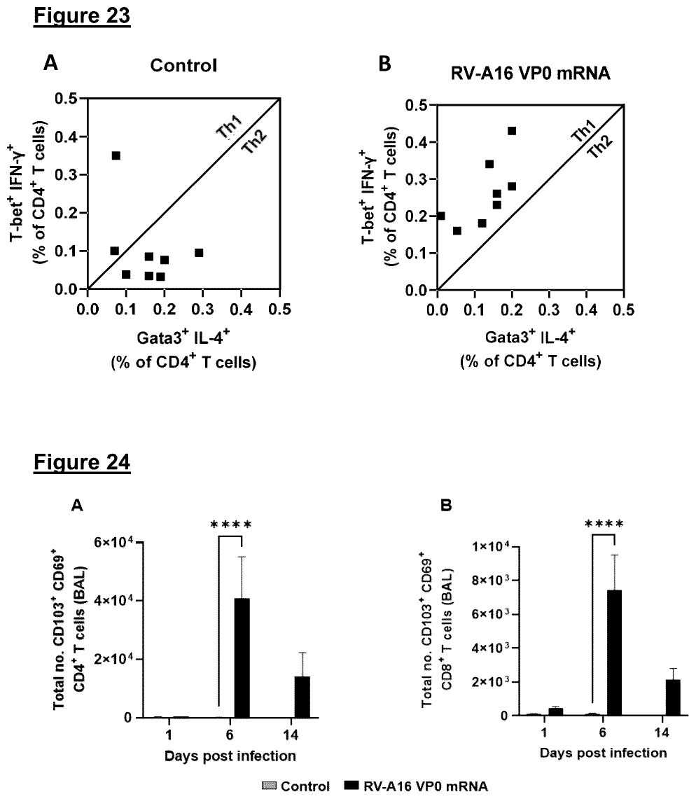

FIG. 23 is a set of correlation plots where the frequency of Th1 cells is plotted against the frequency of Th2 cells for each mouse infected with RV-A1 after immunization with RV-A16 VP0 mRNA (FIG. 23B) or control (FIG. 23A).

FIG. 24 is a set of histograms showing the number of CD4⁺ (FIG. 24A) and CD8⁺ (FIG. 24B) T_{RM} cells in the BAL of control and RV-A16 VP0 mRNA immunized mice at different post-RV-A1 infection timepoints. Bars represent mean cell numbers or mean cell frequency+/−SEM from 8 animals. ****p<0.0001, ANOVA.

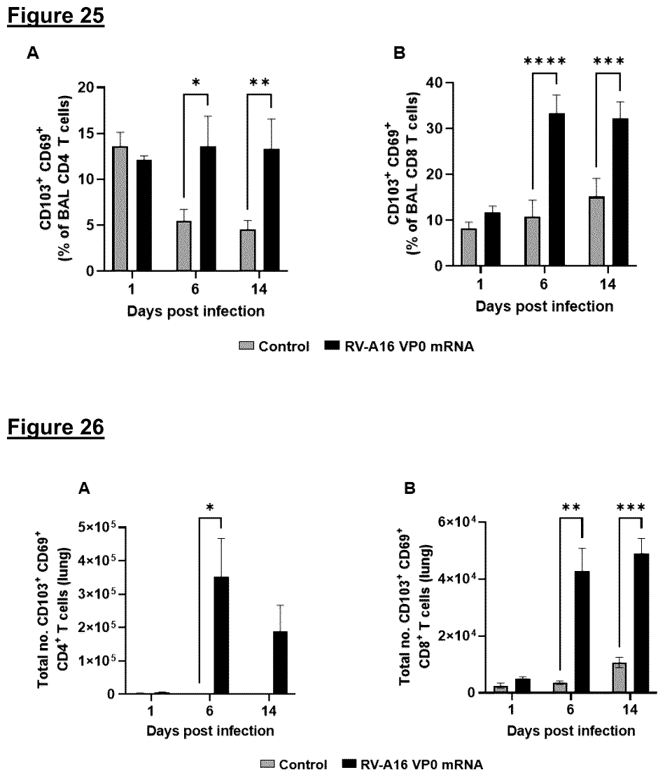

FIG. 25 is a set of histograms showing the frequency of CD4⁺ (FIG. 25A) and CD8⁺ (FIG. 25B) T_{RM} cells in the BAL of control and RV-A16 VP0 mRNA immunized mice at different post-RV-A01 infection timepoints. Bars represent mean cell numbers or mean cell frequency+/−SEM from 8 animals. *p<0.05, **p<0.01, ***p<0.001, ****p<0.0001, ANOVA.

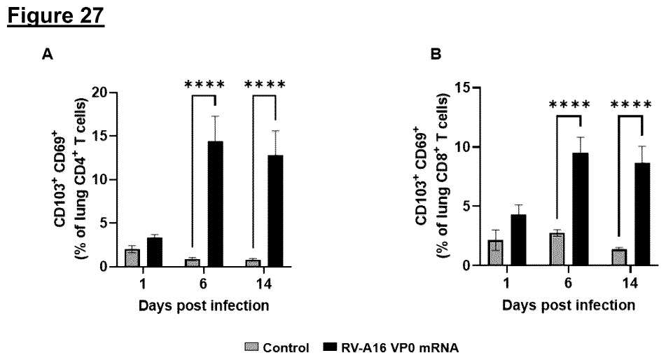

FIG. 26 is a set of histograms showing the number of CD4⁺ (FIG. 26A) and CD8⁺ (FIG. 26B) T_{RM} cells in the lungs of control and RV-A16 VP0 mRNA immunized mice at different post-RV-A1 infection timepoints. Bars represent mean cell numbers or mean cell frequency+/−SEM from 8 animals. *p<0.05, **p<0.01, ***p<0.001, ANOVA.

FIG. 27 is a set of histograms showing the frequency of CD4⁺ (FIG. 27A) and CD8⁺ (FIG. 27B) T_{RM} cells in the lungs of control and RV-A16 VP0 mRNA immunized mice at different post-RV-A1 infection timepoints. Bars represent mean cell numbers or mean cell frequency+/−SEM from 8 animals. ****p<0.0001, ANOVA.

### MATERIALS AND METHODS

### Bioinformatical Analysis to Identify Centroid RV VP0 Immunogens

### RV VP0 Sequences

RV VP0 protein sequences were extracted manually from the NCBI protein database using a mixture of blast and annotation search. The results were refined by comparing to the strains annotated in Taxonomy Browser (NCBI). 150 RV strain VP0 protein sequences were used in the analysis: 78 RV-A, 25 RV-B and 47 RV-C sequences.

### Alignments, Sequence Identity and Similarity Scores

Multiple alignment of the 150 RV VP0 sequences was performed using the MUSCLE (Multiple Sequence Comparison by Log-Expectation) algorithm. From the alignment, identity and similarity matrices were extracted. The identity matrix was used to calculate the percentage of identical amino acids per position between all sequences pairwise. The similarity matrix was used to calculate the percentage of amino acids with similar physicochemical properties per position between all sequences pairwise.

### Hierarchical Clustering

The 150 RV strain VP0 sequences were clustered based on the calculated identity scores (transformed in distances, as required by the method) using non-supervised hierarchical clustering. The tree was then split into three clusters (FIG. 1). From each cluster, a centroid RV VP0 strain sequence (i.e. central/typical strain or medoid) was identified. Each centroid VP0 sequence was defined as the VP0 sequence within that cluster that was predicted to generate an immune response against the maximum percentage of other strains within that cluster. Centroid identification was performed using a range of identify threshold as defined below.

### Fragment Analysis

The conservation among RV strain VP0 protein sequences was also analysed at the epitope level, by splitting each strain VP0 sequence into 12-mers with a one amino acid overlap. Fragment identity was estimated by calculating the percentage of conserved 12-mer fragments between VP0 sequence pairs (i.e. “conserved”” is defined as 100% identity between two fragments). Fragment similarity was estimated by calculating the percentage of similar 12-mer fragments; similar being defined as 80% of the amino acids in the fragments being conserved or evolutionarily “interchangeable” as per BLOSUM62 scoring).

### RV VP0 Protein Production and Purification

TABLE 1
List of plasmids used to produce
the different RV VP0 immunogens.

| Plasmid ID | RV strain VP0 |
|---|---|
| pETSumoRV RVA16 VP0 | RV-A16 |
| pETSumoRV-B-DGW2, VP_D | RV-B06 |
| pETSumoRV-C1 | RV-C01 |
| pETSumoRV-C-DGV2, VP_E | RV-C07 |
| pETSumoRV-C19RV | RV-C19 |
| RV pETSumoRV-C24 | RV-C24 |

The same cloning strategy has been applied for all recombinant proteins. Briefly, each respective nucleotide sequence was optimized for E. coli expression and the synthetic gene was cloned in frame with the SUMO sequence in the T/A cloning site of the pET-SUMO vector and then expressed using the pET-SUMO expression system (Invitrogen). Genes were synthesised and plasmids constructed at Crelux GmbH or Charles River Laboratories.

Escherichia coli (T7 Express, NEB) were transformed with either of the plasmids listed above. Expression was carried out on 1-2 litre scale in shake flasks (Ultra Yield, Thomson) using 2YT media. The cultures were grown to an OD600 nm of 0.8 at 37° C. and protein production was induced by the addition of 1 mM IPTG with the temperature reduced to 18° C. prior to induction. The cells were harvested 16 hours post induction by centrifugation for 10 min at 4,500 g and pellets either stored at −80° C. or processed immediately.

Progression of the purification was monitored by SDS-PAGE after each purification step. Purification was performed at 4° C. using chilled buffers (buffer components are listed in Table 2 below). Pellets were re-suspended in Buffer A (10 mL/1 g of cells) and broken using a cell disrupter (Constant Systems) at 25 kpsi. Lysates were cleared by centrifugation for 30 min at 38,000 g. The resulting inclusion body pellets were washed in the same buffer a further two times and solubilised overnight at 4° C. in Buffer B (10 mL/1 g of cells lysed) by stirring. This solution was then clarified by centrifugation for minutes at 38,000×g and loaded onto two consecutively connected 5 mL HisTrap HP column (Cytiva™) equilibrated in Buffer B at 1 mL/min using an Aktaexpress system. The columns were washed with Buffer B until the baseline was stable which was followed by a 20 CV wash with Buffer C and re-equilibration into Buffer B by a further 20 CV wash. After that a shallow gradient over 30 CV was applied into Buffer D after which the protein was eluted using Buffer E followed by immediate buffer exchange into buffer F using a HiPrep™ 26/10 desalting column (Cytiva™).

To remove the His-SUMO-tag˜0.4 U of Sumo protease (Lifesensors Inc) per 1 mg of VP0 protein were added to the eluted protein and incubated overnight at 4° C.

The cleaved protein was separated from the uncleaved material and protease by secondary nickel chromatography in gravity flow using HisSelect® Affinity Gel (Cytiva™) equilibrated in Buffer F.

The flow through was collected, concentrated to ˜2 mL and loaded onto a Superdex S200 gel filtration column (Cytiva™) equilibrated in Buffer F run at 1 mL/min. Fractions containing VPO protein were pooled for final overnight dialysis into Buffer G.

The dialysed samples were then concentrated between to 0.3-0.6 mg/ml before tested for their Endotoxin levels using the ToxinSensor™ Chromogenic LAL Endotoxin Assay Kit (GenScript®) following the manufacturer's instructions. Samples that had lower levels than 5 EU/mg of protein were aliquoted and stored at −80° C.

TABLE 2
Constituents of buffers used for RV VP0 protein purification

| Buffer | Purification step | Components |
|---|---|---|
| A | Cell lysis | 50 mM Tris pH 8.0, 150 mM NaCl |
| B | Solubilisation | 20 mM Tris pH 8.0, 150 mM NaCl, |
| | | 8M Urea, 10 mM imidazole |
| C | Triton X-114 | 20 mM Tris pH 8.0, 150 mM NaCl, |
| | wash | 8M Urea, 10 mM imidazole, |
| | | 0.2% Triton X-114 |
| D | 2M Urea buffer | 20 mM Tris pH 8.0, 150 mM NaCl, |
| | for gradient | 2M Urea, 10 mM imidazole |
| E | Elution buffer | 20 mM Tris pH 8.0, 150 mM NaCl, |
| | | 2M Urea, 400 mM imidazole |
| F | Desalt/Sizing | 20 mM Tris pH 8.0, 150 mM NaCl, |
| | buffer | 2M Urea |
| G | Dialysis/Refolding | 20 mM Tris pH 8.0, 150 mM NaCl, |
| | buffer | 0.5M arginine |

### Mouse Immunisation

8-10-week-old C57BL/6 mice were immunised by subcutaneous (SC) route on day 0 and day 21.

Each mouse was given 10 μg of RV VP0 protein (either RV-A16, RV-B06, RV-C01, RV-C07, RV-C19 or RV-C24) in a total volume of 100 μl with IFA/CpG adjuvant (10 μg CPG 1826 (Invivogen, Toulouse France)+100 μl Incomplete Freund's Adjuvant (IFA Sigma-Aldrich Gillingham United Kingdom).

Protein buffer (Tris 20 mM, NaCl 150 mM, Arginine 0.5 M pH 8.0) in the presence or absence of IFA/CpG adjuvant was used as a negative control and administered in control groups of mice according to the same procedure.

### Sample Processing

Blood and spleens were collected on day 42. Blood was collected in Vacutainer tubes (BD Vacutainer SST II Nus plastic serum tube (BD Biosciences, Le Pont-De-Claix, France), kept overnight at 4° C. and centrifuged for 20 minutes at 1660 g to separate serum from cells. Sera were stored at −20° C.

Spleens were rapidly collected after sacrifice under sterile conditions.

### Cytokine ELISPOTs

Spleens were homogenized manually with a syringe plunger through a cell strainer (BD Biosciences, San Jose, Calif.) and treated with Red Blood Cell Lysing Buffer HybriMax (Sigma-Aldrich Gillingham, United Kingdom) to lyse red cells. Cells were washed two times with RPMI 1640 medium with HEPES (Gibco, Paisley, UK), supplemented with 2% of decomplemented foetal calf serum (FCS) (HYCLONE Hyclone, Logan, Utah), 50 μM of 2-mercaptoethanol (Gibco), 2 mM of L-glutamine (Gibco) and 100 units/mL of Penicillin-Streptomycin (Gibco). Cells were counted on a Multisizer and resuspended in complete medium with RPMI 1640 medium (Gibco), supplemented with 10% of decomplemented FCS (HYCLONE), 50 μM of 2-mercaptoethanol (Gibco), 2 mM of L-glutamine (Gibco) and 100 units/mL of Penicillin-Streptomycin (Gibco). 2×10⁵ splenocytes per well were seeded in 96 well plates (source) and stimulated with pools of peptides corresponding to the different RV-B06, RV-C01, RV-C07, RV-C19 or RV-C24 full-length VP0 antigens plus murine IL-2 at 20 U/ml. In addition, to assess cross-reactivity the splenocytes were also stimulated with peptide pools corresponding to full length VP0 from a range of RV strains from each RV species, as indicated in each of the figures. Each peptide pool consisted of 80 15-mer peptides covering the entirety of the VP0 protein, overlapping by 11 amino acids. Peptide pools were synthesised by Mimotopes and used at a final concentration of 1 μg/ml. Phorbol 12-myristate 13-acetate (PMA; Invivogen Toulouse France) plus ionomycin (Stemcell, Cambridge United Kingdom) was used as a positive stimulation control. An irrelevant peptide pool (PepMix™ Human (HLA class I Ig-like C1 type domain), sourced from JPT (Berlin Germany), 15-mers with ii amino acid overlap) was used as a negative stimulation control. Plates had been previously coated overnight at 4° C. with rat anti-mouse IFN-y antibody (BD Pharmingen, San Diego, Calif.) at 1 μg per well in sterile PBS and blocked for one hour at 37° C. with 10% FCS complete RPMI 1640 medium (Gibco). Stimulation of splenocytes was performed for 18 hours at 37° C. with 5% CO₂.

After splenocyte stimulation, plates were washed three times with PBS and then three times with PBS-Tween 0.05%. Biotinylated anti-IFN-g (BD Biosciences, Le Pont-De-Claix, France), 100 mL per well, was added at 5 mg/ml in PBS-Tween 0.05% and the plates incubated at 20° C. in the dark. Plates were then washed 3 times with PBS-Tween 0.05% and incubated with streptavidin-horseradish peroxidase (Southern Biotech) in PBS-Tween 0.05% for 1 hour at 20° C. in the dark.

The plates were next washed three times with PBS-Tween 0.05%, followed by three times with PBS. 100 μl per well of substrate solution (3-amino-9-ethylcarbazole, AE, Sigma-Aldrich) was added and the plates incubated for 15 min at 20° C. in the dark. The reaction was stopped by adding approximately 150 μl per well of distilled water. Substrate solution was prepared by mixing: 9 ml distilled water, 1 ml acetate buffer, 0.250 ml AE and 5 μl H₂O₂. The solution was filtered through a 0.22 μm filter (Sigma-Aldrich). Each spot, corresponding to an IFN-y secreting T cell was enumerated with an automatic ELISPOT reader (AID, Strassberg Germany). Results were expressed as number of IFN-y spots per 10⁶ splenocytes.

The ELISPOT data for each immunogen were analysed separately on the log 10 scale using a linear mixed model containing fixed effects for study group and treatment group and a random effect adjusting for individual mice. Results for each treatment group were reported on the back transformed (anti-logged) scale using least square geometric means, ratios of least square geometric means, 95% confidence intervals and p-values for individual comparisons. Statistical significance was reported at 5% significance level (i.e. p<0.05). No multiple comparison adjustment was performed as all comparisons were defined a priori and were against the control group (irrelevant peptide).

### ELISA

Anti-RV VP0 IgG2a (or IgG2c responses in 57B1/6 mice) responses were measured by ELISA. Greiner 96-well microplates (Greiner, Gloucestershire, United Kingdom) were coated with 100 ng per well of RV-B06, C19 or C24 VP0 in PBS buffer pH 7.4 (Sigma-Aldrich) overnight at 4° C. Non-specific sites were blocked with 150 μl per well of PBS-Tween (PBS pH 7.1, 0.05% Tween 20) plus 1% skimmed milk for one hour at 37° C. Sera were serially diluted 2-fold in the VP0 coated plates from starting dilutions of 1:100 or 1:1000 in PBS-Tween 0.05%, milk 1%, incubated for 90 minutes and washed three times with PBS-Tween.

RV VP0-specific IgG2a was detected by adding goat anti-mouse IgG2a conjugated to HRP (Southern Biotech, Birmingham, Ala.) diluted 1:4000 in PBS-Tween plus 1% skimmed milk to each well and incubating for 90 minutes at 37° C.

The plates were then washed three times with PBS-Tween and TetraMethylBenzidine (TMB, Tebu-bio laboratories, Le Perray-en-Yvelines, France) substrate solution added to each well at 100 mL per well. The plates were incubated for 10-30 minutes in the dark at room temperature. The colorimetric reaction was stopped by the addition off 100 μl/well of 1M HCl (VWR Prolabo Fontenay-sou-Bois, France). The plates were immediately read at 450 and 650 nm on a Versamax plate reader (Molecular Devices).

In Vitro mRNA Studies
Transfection of HEK Cells with VP0 mRNA & Detection of Expressed RV VP0 Proteins

HEK293 cells were cultured in DMEM medium supplemented with 10% FCS and 1% penicillin/streptomycin. For transfection, cells were plated in 24 well plates and transfected in duplicate at approximately 80% confluency. Cells were transfected with RV VP0 mRNA with mRNA-Fect (RJH Biosciences) according to a pre-optimised protocol consisting of 4 μL per well of mRNA (Tri-Link), 60 μL of serum free DMEM medium and 4 μL of mRNA-fect. An untransfected control was included and consisted of mRNA-fect without any mRNA. Prior to transfection, each mRNA was diluted to 0.1 mg/mL in RNase free water (Promega) and mRNA-fect was used as a neat solution of 1 mg/mL according to the manufacturer's recommended protocol. Diluted mRNA (4 μL) was added to 60 μL DMEM medium and vortexed for 3 sec. Undiluted mRNA-fect (4 μL per transfection), was then added and the RNA mixtures vortexed for 10 sec and then left at room temperature for 30 min to complex. After incubation, the complexes (68 μL in total) were added dropwise directly to each well of a 24 well plate in duplicate, in which the medium volume was 0.5 mL. Cells were then incubated for a set period of time (24-96 h) prior to harvest. For sample harvests, the medium was removed, monolayers washed in 0.5 mL PBS pH 7.4, and 100 μL of 2×SDS sample buffer (Invitrogen) added over the two duplicate wells. The cells were lysed by both pipetting and scraping, duplicates were then pooled, producing 1 sample and samples were stored at −80° C. Protein expression was visualised using SDS-polyacrylamide gel electrophoresis (SDS-PAGE, Invitrogen) followed by transfer of proteins onto nitrocellulose (Invitrogen), and western blotting. Western blotting consisted of probing each membrane with pooled VP0 specific mouse anti-sera (anti-A16, B06 or C24 VP0) diluted 1/200 in blocking buffer (5% skim milk in PBS) overnight at 4° C. In certain experiments, the membranes were probed with an anti-RV-A16 monoclonal antibody (Antibodies Online, Cat No. ABIN1000236) diluted 1/500 in blocking buffer overnight. Following incubation, membranes were washed 3× in 30-50 mL washing buffer (PBS pH 7.4 with 0.1% Tween 20) for 5 min with shaking. After washing, an anti-mouse H+L chain IgG-HRP secondary antibody (Invitrogen) was added diluted 1/5,000 in blocking buffer. The secondary antibody was incubated for 1 h with shaking, the secondary removed, and 30-50 mL of washing buffer added for 30 min, followed by 2× washes of 30-50 mL washing buffer for 5 min each. After washing, 2 mL of ECL Plus substrate (Thermo Scientific) was added for 2 min and the membranes subjected to image acquisition using a Peqlab imager according to the manufacturer's recommended protocol for chemiluminescence.

Cynomolgus Monkey mRNA Vaccine Studies

All studies were performed under local laws and regulations and were reviewed and approved by the Institutional Animal Care and Use Committee (IACUC).

mRNA Vaccine Preparation

On the day of use, in vivo-jetRNA® transfection reagent and mRNA buffer (both Polyplus, France) were equilibrated to room temperature. mRNA was diluted in mRNA buffer to 50 μg/mL. A volume of in vivo-jetRNA® transfection reagent equal to the volume of mRNA added to the mRNA buffer was added and the resulting solution was incubated for 15 minutes at room temperature prior to use. For control solutions, the mRNA was replaced with an equal volume of sterile water (Polyplus).

### Cynomolgus Monkey Immunization

Each animal received 100 μg of mRNA in a total volume of 2 mL, or 2 mL transfection control reagent, dosed intramuscularly on Days 0 and 21 of the study.

### Blood Sampling and Preparation of Plasma and Peripheral Blood Mononuclear Cells

Blood was collected on Days 0, 14, 21, 28, 35 and 42 of the study from the saphenous or cephalic veins. The blood was incubated at room temperature for 30 minutes before being centrifuged and plasma and peripheral blood mononuclear cells (PBMCs) prepared by standard methods.

### Plasma ELISA

96-well plates were coated with Goat Anti-Rhesus IgG(H+L)-UNLB (Southern Biotech, U.S.) diluted in PBS for the standard curve, or 25 μg/well RV VP0 protein diluted in PBS for detection of antigen-specific IgG in monkey plasma samples. Plates were incubated overnight at 4° C. Plates were washed three times with PBS-Tween (PBS containing 0.05% Tween 20 (Sigma-Aldrich)). Non-specific binding was blocked by incubation with PBS 5% milk (Sigma-Aldrich) for 2 hours at room temperature. Plates were washed three times with PBS-Tween. 100 μL standard or plasma sample were added per well, diluted in PBS-1% BSA and incubated for 2 hours at room temperature. An 11-point standard curve was generated using serial 2-fold dilutions of Rhesus Monkey IgG-UNLB (Southern Biotech, U.S) starting from 400 ng/mL. Plates were washed three times with PBS-Tween. Bound antibody was detected with Peroxidase AffiniPure Goat Anti-Human IgG, Fey fragment specific (Jackson Immunoresearch, U.S.) diluted 1:5000 in PBS 1% BSA (Sigma-Aldrich), incubated for 1 hour at room temperature. Plates were washed three times with PBS-Tween, then developed using 100 μL TMB substrate per well incubated for approximately 10 minutes, followed by 100 μL 0.18M H₂SO₄ per well to stop the reaction. Plates were read immediately at 450 nm using a SpectraMax plate reader (Molecular Devices).

### PBMC ELISpot

PBMCs were stimulated with peptide pools as described above using 2×10⁵ cells per peptide pool stimulation. Each stimulation was performed in triplicate. IFN-γ or IL-4 ELISpot was performed using Monkey IFN-γ ELISpot PRO kit (ALP), strips (Mabtech-3421M-2AST-10) and Human IL-4 ELISpot PRO kit (ALP) (Mabtech-3410-2APW-10), according to the manufacturers instructions. The activity per 2×10⁵ PBMCs was determined using an AID ELISpot machine (Model ELR088IFL).

Mouse mRNA Vaccine Studies
mRNA Vaccine Preparation

On the day of use, in vivo-jetRNA® transfection reagent and mRNA buffer (both Polyplus, France) were equilibrated to room temperature. mRNAs were diluted in mRNA buffer to 200 μg/mL. An equal volume of in vivo-jetRNA® transfection reagent was added to the diluted mRNA, and the resulting solution was incubated for 15 minutes at room temperature prior to use. For control solutions, the mRNA was replaced with an equal volume of sterile water (Polyplus).

### Mouse Immunisation

Each mouse received 5 μg RV mRNA vaccine in a total volume of 50 μL, or 50 μL transfection control reagent, dosed intramuscularly on Days 0 and 21 of the study.

### Mouse Rhinovirus Infection

Each mouse was dosed intranasally (i.n.) with 50 μL rhinovirus preparation (grown as described in Bartlett et al, 2008, Nat. Med.) or 50 μL PBS (ThermoFisher Scientific, UK), administered dropwise to both nares whilst under isofluorane anaesthesia.

### Mouse Sample Harvesting and Processing

Blood samples were obtained at timepoints prior to sacrifice via tail vein puncture and collected using Na-heparinised capillary tubes (Hirschmann-Laborgeräte, Germany). Terminal blood samples were collected from the jugular vein using Na-heparinised capillary tubes. Whole blood samples were centrifuged to isolate serum from cells, and serum was subsequently stored at −80° C.

Following euthanasia, the trachea was cannulated and bronchoalveolar lavage (BAL) was performed using three 0.5 mL sterile PBS washes per mouse, pooled to give a total volume of 1.5 mL BAL. Samples were centrifuged at 1500 RPM for 10 min at 4° C. and supernatants were stored at −80° C. Cells were re-suspended in 0.2% w/v NaCl to haemolyse red blood cells, followed by an equal volume of 1.6% w/v NaCl.

Lymph nodes and spleens were harvested into Rio medium (RPMI 1640 medium containing 10% foetal calf serum (FCS) and 100 U/mL penicillin/streptomycin (P/S), all ThermoFisher Scientific), then homogenised manually through 100 μm filters (Greiner Bio-One, UK). Red blood cells were lysed using ACK Lysing Buffer (Fisher Scientific, UK) and cells were then washed twice with Rio medium.

The superior right lung lobe and post caval lung lobe from each mouse were harvested into RNAprotect (Qiagen, UK) and stored at −80° C. The remaining lung lobes were collected into Rio medium then diced finely and incubated at 37° C. for 30 minutes in Rio medium supplemented with 0.14 Wunsch units/mL Liberase (Roche, UK) and 50 μg/mL DNase1 (Roche). Lung tissue was then manually homogenised through 100 μm filters (Greiner Bio-One) Red blood cells were lysed using ACK Lysing Buffer (Fisher Scientific, UK) and cells were then washed twice with Rio medium.

Viability of single cell preparations was assessed using trypan blue (ThermoFisher Scientific) exclusion staining and total cells were enumerated using a haemocytometer (Neubauer, Germany).

### Neutralisation Assay Using Mouse Serum

Mouse sera were serially diluted 1 in 2 or 1 in 3.14 and incubated with RV-A1 (5×10⁴ TCID50 per mL) in an 8-point titration curve for 1 h with shaking. The virus-serum complexes (50 mL per well) were then incubated with 150 mL per well of HeLa cells (3×10⁵ cells per mL) in replicates of eight in flat bottomed 96 well plates. Cells with virus, and cells only were used as controls. In some experiments, anti-RV-A1 guinea pig sera (ATCC Cat No V-113-501-558) was used as a positive control for neutralisation through serial 1 in 3.14 dilutions creating a 12-point titration curve. Plates were incubated for 3 days and then supernatants were discarded, and cell monolayers stained with 150 ml per well crystal violet solution (0.1% in PBS) for 10-15 min, the stain was then removed by gentle rinsing under a tap. Stained monolayers were then incubated with 150 mL per well of 1% SDS solution in PBS to solubilise the stain, over a 2 h incubation period on an orbital shaker at room temperature. The resulting optical density was measured at 560 nm using a SpectraMax plate reader (Molecular Devices, U.S.).

### Lung Virus Load

RNA was extracted from whole lung tissue using RNeasy® mini kit columns according to manufacturers' instructions and converted to cDNA using Omniscript® Reverse Transcription Kit (both Qiagen, UK). RV was quantified relative to 18S rRNA expression using a 50 nM forward primer (5′-tgagtcctccggcccctgaatg-3′), 300 nM forward primer (5′-gtgaagagccscrtgtgct-3′) and 5 μM QuantiTect Probe (Qiagen) and interpolated using an RV plasmid standard generated in house. Quantitative rtPCR was performed using a QuantStudio 5 real time qPCR machine (ThermoFisher Scientific).

### Ex Vivo Cell Stimulations for Flow Cytometry

Single cell suspensions of lung or BAL cells were stimulated for 4 hours at 37° C. in Rio medium containing 40 ng/mL PMA (Invivogen, U.S.) and 3 μg/mL ionomycin (Stemcell Technologies, Canada), or 16 μg/mL RV peptide pools, in the presence of 10 μg/mL brefeldin A (Enzo Life Sciences, U.S.). Unstimulated control samples were incubated in Rio medium containing brefeldin A alone.

### Flow Cytometry

Single cell suspensions of lung, BAL or lymph node cells were initially stained with Fixable Violet Dead Cell Stain Kit (Invitrogen, US), then stained with anti-mouse surface marker antibodies plus TruStain FcX antibody diluted in FACS buffer (PBS containing 2% FCS and 2 mM EDTA (Fisher Scientific)) for 30-60 min. Cell were washed twice with FACS buffer and fixed with a 1% formaldehyde solution (Sigma-Aldrich, UK) in FACS buffer. For intranuclear staining, cells were fixed with Transcription Factor Staining Buffer Set (eBioscience, US) and stained with anti-mouse transcription factor and cytokine antibodies diluted in permeabilisation buffer (eBioscience) for 1 hour. Samples were acquired using an LSRFortessa (BD, US) and analysed using FlowJo™ (BD).

### ELISA

Nunc Maxisorp 96-well plates (Fisher Scientific) were coated with 50 ng/well mouse anti-IgG antibody (R&D Systems, U.S.) diluted in PBS for the standard curve, and 25 μg/well RV-A16, RV-B06 or RV-C24 protein diluted in PBS for detection of antigen-specific IgG in mouse serum samples. Plates were incubated overnight at 4° C. Plates were washed three times with PBS-Tween (PBS containing 0.05% Tween 20 (Sigma-Aldrich)). Non-specific binding was blocked by incubation with PBS 5% milk (Sigma-Aldrich) for 1 hour at room temperature. Plates were washed three time with PBS-Tween. 50 μL standard or serum sample were added per well, diluted in PBS 5% milk and incubated for 2 hours at room temperature. A 7-point standard curve was generated using serial 1:3 dilutions of Total IgG mouse ELISA standard (Invitrogen) starting from 100 μg/mL. Plates were washed three times with PBS-Tween. Bound antibody was detected with 25 ng/well Mouse IgG Biotinylated Antibody (R&D Systems) diluted in PBS 1% BSA (Sigma-Aldrich), incubated for 1 hour at room temperature. Plates were washed three times with PBS-Tween, then incubated with 50 ng/well Streptavidin-HRP conjugate (Millipore, U.S.) for 20 minutes at room temperature in the dark. Plates were washed three times with PBS-Tween, then developed using 50 μL TMB substrate (Fisher Scientific) per well incubated for approximately 5 minutes, followed by 50 μL 0.18M H₂SO₄ per well to stop the reaction. Plates were read immediately at 450 nm using a Petromax plate reader (Molecular Devices).

### Results

### Identification of Centroid RV Sequences to Use as Cross-Protective Immunogens

### Defining Thresholds for Centroid Identification

A study by Glanville et al (2013) [6], demonstrated that immunisation of mice with the VP0 protein from RV-A16 generated cellular immunity that cross-reacted with RV-B14, RV-A1 and RV-A29. Identity scores for RV-B14, RV-A1 and RV-A29 VP0 sequences as compared pairwise to the RV-A16 VP0 sequence were calculated (Table 3).

TABLE 3
Sequence and fragment identity and similarity scores from MUSCLE
analysis of 150 RV strain VP0 protein sequences used to demonstrate
cross-immunisation in the Glanville et al. (2013) study. The
threshold scores are highlighted in bold.

| | Score (%) | | | |
|---|---|---|---|---|
| | RV-A16 | RV-B14 | RV-A1 | RV-A29 |
| Matrix | VP0 | VP0 | VP0 | VP0 |
| Sequence identity | 100 | 59.9 | 87.7 | 82.7 |
| to RV-A16 VP0 | | | | |
| Sequence similarity | 100 | 84.0 | 94.6 | 92.5 |
| to RV-A16 VP0 | | | | |
| Fragment identity | 100 | 3.1 | 48.3 | 29.6 |
| to RV-A16 VP0 | | | | |
| Fragment similarity | 100 | 72.9 | 90.0 | 86.1 |
| to RV-A16 VP0 | | | | |

Glanville et al (2013) [6] demonstrated that RV-A16 VP0 protein immunisation of mice induces cellular immunity that cross-reacts with RV-A14, RV-A1 and RV-A29. The lowest identity and similarity scores were used as the thresholds: any VP0 proteins with sequence identity scores of >59.9% or similarity scores of >84.0% (Table 3) were predicted to generate cross-reactive cellular immunity against each other. Any VP0 proteins with identity scores below these thresholds were not predicted to generate cross-reactive immunity to each other.

The thresholds were used to select centroid VP0 proteins from each cluster.

### Identification of Centroids

Identification of centroids was first performed using sequence identity scores only as this is the most rigid approach.

FIG. 1 is a heatmap of the MUSCLE computed sequence identity scores between the 150 RV strain VP0 sequences. Those sequences with an identity score >59.9% are shown in blue. RV-A16 VP0 has previously been demonstrated as a potential centroid VP0 sequence for RV-A species. This data confirms that RV-A16 VP0 sequence has identity scores >59.90% against all other RV-A species strains and is therefore predicted to induce cellular immunity against all other A strains and was selected as the RV-A species centroid immunogen.

However, the heatmap demonstrates that RV-A16 VP0 is not predicted to cross-immunise against many RV-B and C species strains. Therefore, to identify additional RV VP0 protein centroids that are predicted to induce cellular immunity against all other strains, hierarchical clustering based upon average linkage was performed. FIG. 2 is a circular tree created by hierarchical clustering of 140 RV strain VP0 sequences based on identity scores and split into three clusters. Cluster 1 (red) contains all RV-A species sequences, cluster 2 (black) contains all RV-B species strains and cluster 3 (green) contains all RV-C strains included in the analysis. Based upon this analysis RV-B06 VP0 was selected as the RV-B species centroid as it has identity scores against all other RV-B species VP0 protein sequences above the threshold of 59.9% (Table 4). The RV-C species centroid was identified as RV-C07, as this VP0 sequence was calculated to cover all other RV-C species VP0 protein sequences above the threshold of 59.9% (Table 4).

TABLE 4
Percentage of RV strains within cluster 2 (n = 25)
and cluster 3 (n = 47) predicted to be cross-immunised
by RV-B06 VP0 or RV-C07 VP0 at different sequence identity
thresholds. The required sequence identity threshold predicted
for cross-immunisation is highlighted in bold.

| | |
|---|---|
| | % of strains within the cluster |
| | predicted to be cross-immunised |
| Sequence | by the centroid |

| identity score | Cluster 2 - | Cluster 3 - |
|---|---|---|
| threshold | RV-B06 VP0 | RV-C07 VP0 |
| 30% | 100 | 100 |
| 40% | 100 | 100 |
| 50% | 100 | 100 |
| 60% | 100 | 100 |
| 70% | 100 | 100 |
| 80% | 87.01 | 32.61 |
| 90% | 11.69 | 4.35 |
| 95% | 1.3 | 2.17 |

### Identification of Additional RV-C Species Centroids

The inventors then investigated the number of IFN-γ producing splenocytes from mice immunised with RV-A16 VP0 or RV-B06 VP0 protein. The data presented in FIGS. 3 and 4 demonstrates that, when used as immunogens in mice, RV-A16 VP0 and RV-B06 VP0 proteins, respectively, induce cellular cross-immunity against all other RV-A and RV-B species, respectively (FIGS. 3B and 4B). However, neither RV-A16 or RV-B06 VP0 immunization induced cellular immunity to RV-C species (FIGS. 3C and 4C). When RV-C07 VP0 was used as an immunogen, the splenocytes from mice immunised with RV-C07 responded weakly to stimulation with peptide pools from other RV-C species strains (FIG. 5B), compared to the responses of splenocytes from RV-A16 and RV-B06 immunised mice to stimulation with peptides from their respective species (FIGS. 3B and 4B). Furthermore, there was very little cross-reactivity with peptide pools from RV-A and B species strains (FIG. 5C). Therefore, further analysis was performed to select new RV-C species VP0 immunogens that would provide good cross-immunity against other RV-C species, but were dissimilar to RV-C07.

A deeper level of analysis was required to identify RV-C centroids that were dissimilar from RV-C07, but still predicted to cross-react with all other RV-C species strains. Therefore, the new analysis was performed using all scores calculated: sequence identity, sequence similarity, fragment identity and fragment similarity.

The identity and similarity scores were aggregated as the geometric mean of the logged measures (+1 was added before taking the log to avoid negative values). This value was referred to as the identity/similarity score, with a higher score indicating higher similarity and identity. Two identity/similarity scores were reported, one for the whole VP0 sequence analysis, and one for the fragment analysis: the sequence identity/similarity scores were used to compare against RV-C07VP0, and the fragment identity/similarity scores were used to predict the potential for cross-immunisation based upon the thresholds calculated previously.

In FIG. 6, the median fragment identity/similarity score for each RV-C strain VP0 sequence across all other RV-C VP0 strains was plotted against the median sequence identity/similarity score for each strain against RV-C07 alone. When all RV-C species strains were analysed as a single cluster (FIG. 6A), RV-C24, C25 and C49 VP0s had the highest median fragment identity/similarity score across all other RV-C species strains tested, while having a sequence identity/similarity score that was different from RV-C07. When the RV-C species strains were split into two clusters (FIG. 6B) RV-C19 VP0, was identified as a potential centroid from the 1^{st} cluster and RV-C01 VP0 from the second cluster.

When the VP0 sequences identified were compared using the non-aggregated sequence identities and similarities to RV-C07VP0 and the non-aggregated identities and sequences to all RV-C strains VP0 (Table 5), RV-C24 VP0 was selected as the centroid RV-C species VP0, having the best overall balance between predicted cross-strain coverage and dissimilarity to RV-C07 VP0. From the two-cluster analysis, RV-C01 and RV-C19 VP0 were selected for further testing.

TABLE 5
Sequence identity and similarity scores to RV-C07 and fragment identity
and similarity scores across all other RV-C species strains tested
(n = 47). The centroids selected are highlighted in bold.

| | RV-C24 | RV-C25 | RV-C49 | RV-C19 |
|---|---|---|---|---|
| | VP0 | VP0 | VP0 | VP0 |
| Sequence identity to | 79.9 | 75.3 | 80.2 | 73.3 |
| RV-C07 VP0 (%) | | | | |
| Sequence similarity to | 93.3 | 92.2 | 93.6 | 91.2 |
| RV-C07 VP0 (%) | | | | |
| Fragment identity to | 14.2 | 11.2 | 14.2 | 5.7 |
| all RV-C strains VP0 (%) | | | | |
| Fragment similarity to | 86.2 | 86.9 | 87.1 | 89.3 |
| all RV-C strains VP0 (%) | | | | |

### RV-A16 VP0 Immunisation

FIG. 3 is a set of histograms that show the results from ELISPOT experiments to analyse the cellular immunity evoked by immunisation with RV-A16 VP0 protein against VP0 from strains of other RV species. Mice were immunised twice with RV-A16 VP0 protein with adjuvant and the spleens subsequently harvested, as described above. To quantify the level of cellular immunity evoked by immunisation with RV-A16 VP0, splenocytes were isolated and incubated with different stimuli, as indicated in the FIG. 3. Splenocytes that are activated by the stimulus secrete the cytokine IFN-γ and the degree of IFN-γ secretion was quantified by ELISPOT analysis.

The results show that splenocytes from RV-A16 VP0 immunised mice secrete IFN-γ in response stimulation with VP0 peptide pools from a diverse set of A species RVs (FIG. 3B). This is in addition to IFN-γ secretion in response to the restimulation with the same recombinant RV-A16 VP0 protein used to immunise (FIG. 3A).

When splenocytes from RV-A16 VP0 immunised mice were stimulated with nothing (medium alone) or an irrelevant peptide, there was negligible production of IFN-γ by the splenocytes (FIGS. 3A and 3B). When the degree of IFN-γ secretion by splenocytes in response to RV-A species peptide pools was statistically compared to that produced in response to stimulation with the irrelevant peptide, the results for all peptide pools tested were highly significant (FIG. 3B). These results demonstrate that immunisation of mice with RV-A16 VP0 evokes cellular immunity that is cross-reactive with other members of the A species of RVs. However, when splenocytes from RV-A16 immunised mice were stimulated with peptide pools corresponding to VP0 proteins from RV-B and C species strains, no significant secretion of IFN-γ was observed (FIG. 3C). This indicates that RV-A16 immunisation does not induce cross-reactive cellular immunity to strains outside of RV-A species.

### RV-B06 VP0 Immunisation

FIG. 4 is a set of histograms that show the results from ELISPOT experiments to analyse the cellular immunity evoked by immunisation with RV-B06 VP0 protein. Mice were immunised twice with RV-B06 VP0 protein with adjuvant and the spleens subsequently harvested, as described above. To quantify the level of cellular immunity evoked by immunisation with RV-B06 VP0, splenocytes were isolated and incubated with different stimuli, as indicated in FIG. 4. Splenocytes that are activated by the stimulus secrete the cytokine IFN-γ and the degree of IFN-γ secretion was quantified by ELISPOT analysis.

The results show that splenocytes from RV-B06 VP0 immunised mice secrete IFN-γ in response stimulation with VP0 peptide pools from a diverse set of B species RVs (FIG. 4B). This is in addition to IFN-γ secretion in response to the immunising RV-B06 VP0 protein (FIG. 4A) and the RV-B06 peptide pool (FIG. 4B).

When splenocytes from RV-B06 VP0 immunised mice were stimulated with nothing (medium alone) or an irrelevant peptide, there was negligible production of IFN-γ by the splenocytes (FIGS. 4A and 4B). When the degree of IFN-γ secretion by splenocytes in response to RV-B species peptide pools was statistically compared to that produced in response to stimulation with the irrelevant peptide, the results for all peptide pools tested were highly significant (FIG. 4B).

These results demonstrate that immunisation of mice with RV-B06 VP0 evokes cellular immunity that is cross-reactive with other members of the B species of RVs. However, when splenocytes from RV-B06 immunised mice were stimulated with peptide pools corresponding to VP0 proteins from RV-A and C species strains, significant secretion of IFN-γ was only observed in response to peptides from some RV-A species strains (FIG. 4C). This indicates that RV-B06 VP0 immunisation induces limited cross-reactive cellular immunity to RV-A species strains, and none to RV-C species.

To further demonstrate that immunisation of mice with RV-B06 VP0 induces immunity, the humoral response to the immunising antigen was assessed. Mice were immunised twice with RV-B06 VP0 protein with adjuvant and the serum subsequently harvested, as described above. The quantity of RV-B06 VP0 specific antibodies in the mouse serum was determined by ELISA. FIG. 10 is a set of line graphs that shows the levels of anti-RV-B06 VP0 antibodies in serum from mice following immunisation, as quantified by ELISA.

In all mice immunised with RV-B06 VP0, there are high levels of antibody that specifically recognise RV-B06 VP0, while in control immunised mice there are no discernible levels. These results demonstrate that, in addition to the induction of cross-reactive cellular immunity by immunisation with RV-B06 VP0, there is also a strong humoral response to the immunising protein.

### RV-C07 VP0 Immunisation

FIG. 5 is a set of histograms that show the results from ELISPOT experiments to analyse the cellular immunity evoked by immunisation with RV-C07 VP0 protein against VP0 peptides from other strains of RV. Mice were immunised twice with RV-C07 VP0 protein with adjuvant and the spleens subsequently harvested, as described above. To quantify the level of cellular immunity evoked by immunisation with RV-C07 VP0, splenocytes were isolated and incubated with different stimuli, as indicated in FIG. 5. Splenocytes that are activated by the stimulus secrete the cytokine IFN-γ and the degree of IFN-γ secretion was quantified by ELISPOT analysis.

The results show that while splenocytes from RV-C07 VP0 immunised mice secrete IFN-γ when re-stimulated with the RV-C07 VP0 immunogen (FIG. 5A), there is less significant IFN-γ secretion in response to stimulation with peptide pools corresponding to other RV-C species strains (FIG. 5B). Furthermore, there was very little cross-reactivity to VP0 peptides from other RV species (FIG. 5C). As a result, deeper bioinformatical analysis was performed to identify other VP0 sequences that could act as centroid immunogens to cover RV-C species strains.

### RV-C01 VP0 Immunisation

FIG. 7 is a set of histograms that show the results from ELISPOT experiments to analyse the cellular immunity evoked by immunisation with RV-C01 VP0 protein against other strains of RV. Mice were immunised twice with RV-C01 VP0 protein with adjuvant and the spleens subsequently harvested, as described above. To quantify the level of cellular immunity evoked by immunisation with RV-C01 VP0, splenocytes were isolated and incubated with different stimuli, as indicated in FIG. 7. Splenocytes that are activated by the stimulus secrete the cytokine IFN-γ and the degree of IFN-γ secretion was quantified by ELISPOT analysis.

The results show that splenocytes from RV-C01 VP0 immunised mice secrete IFN-γ in response to stimulation with VP0 peptide pools from a diverse set of C species RVs (FIG. 7B). This is in addition to IFN-γ secretion in response to the immunising RV-C01 VP0 protein (FIG. 7A) and peptide pool (FIG. 7B).

When splenocytes from RV-C01 VP0 immunised mice were stimulated with nothing (medium alone; FIG. 7A) or an irrelevant peptide (FIG. 7B), there was negligible production of IFN-γ by the splenocytes. When the degree of IFN-γ secretion by splenocytes in response to RV-C species peptide pools was statistically compared to that produced in response to stimulation with the irrelevant peptide, the results for all peptide pools tested, except RV-C19 VP0 peptides, were highly significant (FIG. 7B).

These results demonstrate that immunisation of mice with RV-C01 VP0 evokes cellular immunity that is cross-reactive with other members of the C species of RVs. Furthermore, when splenocytes from RV-C01 immunised mice were stimulated with VP0 peptide pools from strains of other RV-species, there was significant IFN-γ secretion in response to peptides from both RV-A and B species (FIG. 7C).

To further demonstrate that immunisation of mice with RV-C01 VP0 induces immunity, the humoral response to the immunising antigen was assessed. Mice were immunised twice with RV-C01 VP0 protein with adjuvant and the serum subsequently harvested, as described above. The quantity of RV-C01 VP0 specific antibodies in the mouse serum was determined by ELISA. FIG. 10 is a set of line graphs that shows the levels of anti-RV-C01 VP0 antibodies in serum from mice following immunisation, as quantified by ELISA.

In all mice immunised with RV-C01 VP0 there are high levels of antibody that specifically recognises RV-C01 VP0, while in control immunised mice there are no discernible levels. These results demonstrate that, in addition to the induction of cross-reactive cellular immunity by immunisation with RV-C01 VP0, there is also a strong humoral response to the immunising protein.

### RV-C19 VP0 Immunisation

FIG. 8 is a set of histograms that show the results from ELISPOT experiments to analyse the cellular immunity evoked by immunisation with RV-C19 VP0 protein against other strains of RV. Mice were immunised twice with RV-C19 VP0 protein with adjuvant and the spleens subsequently harvested, as described above. To quantify the level of cellular immunity evoked by immunisation with RV-C19 VP0, splenocytes were isolated and incubated with different stimuli, as indicated in FIG. 8. Splenocytes that are activated by the stimulus secrete the cytokine IFN-γ and the degree of IFN-γ secretion was quantified by ELISPOT analysis.

The results show that splenocytes from RV-C19 VP0 immunised mice secrete IFN-γ in response to stimulation with VP0 peptide pools from a diverse set of C species RVs (FIG. 8B). This is in addition to IFN-γ secretion in response to the immunising RV-C19 VP0 protein (FIG. 8A) and peptide pool (FIG. 8B).

When splenocytes from RV-C19 VP0 immunised mice were stimulated with nothing (medium alone; FIG. 8A) or an irrelevant peptide (FIG. 8B), there was negligible production of IFN-γ by the splenocytes. When the degree of IFN-γ secretion by splenocytes in response to RV-C species peptide pools was statistically compared to that produced in response to stimulation with the irrelevant peptide, the results for all peptide pools tested were highly significant (FIG. 8B).

These results demonstrate that immunisation of mice with RV-C19 VP0 evokes cellular immunity that is cross-reactive with other members of the C species of RVs. Furthermore, when splenocytes from RV-C19 immunised mice were stimulated with VP0 peptide pools from strains of other RV-species, there was significant IFN-γ secretion to in response to peptides from both RV-A and B species (FIG. 8C).

To further demonstrate that immunisation of mice with RV-C19 VP0 induces immunity, the humoral response to the immunising antigen was assessed. Mice were immunised twice with RV-C19 VP0 protein with adjuvant and the serum subsequently harvested, as described above. The quantity of RV-C19 VP0 specific antibodies in the mouse serum was determined by ELISA. FIG. 10 is a set of line graphs that shows the levels of anti-RV-C19 VP0 antibodies in serum from mice following immunisation, as quantified by ELISA.

In all mice immunised with RV-C19 VP0 there are high levels of antibody that specifically recognises RV-C19 VP0, while in control immunised mice there are no discernible levels. These results demonstrate that, in addition to the induction of cross-reactive cellular immunity by immunisation with RV-C19 VP0, there is also a strong humoral response to the immunising protein.

### RV-C24 VP0 Immunisation

FIG. 9 is a set of histograms that show the results from ELISPOT experiments to analyse the cellular immunity evoked by immunisation with RV-C24 VP0 protein against other strains of RV. Mice were immunised twice with RV-C24 VP0 protein with adjuvant and the spleens subsequently harvested, as described above. To quantify the level of cellular immunity evoked by immunisation with RV-C24 VP0, splenocytes were isolated and incubated with different stimuli, as indicated in FIG. 9. Splenocytes that are activated by the stimulus secrete the cytokine IFN-γ and the degree of IFN-γ secretion was quantified by ELISPOT analysis.

The results show that splenocytes from RV-C24 VP0 immunised mice secrete IFN-γ in response to stimulation with VP0 peptide pools from a diverse set of C species RVs (FIG. 9B). This is in addition to IFN-γ secretion in response to the immunising RV-C24 VP0 protein (FIG. 9A) and the RV-C24 peptide pool (FIG. 9B).

When splenocytes from RV-C24 VP0 immunised mice were stimulated with nothing (medium alone; FIG. 9A) or an irrelevant peptide (FIG. 9B), there was negligible production of IFN-γ by the splenocytes. When the degree of IFN-γ secretion by splenocytes in response to RV-C species peptide pools was statistically compared to that produced in response to stimulation with the irrelevant peptide, the results for all peptide pools tested were highly significant (FIG. 9B).

These results demonstrate that immunisation of mice with RV-C24 VP0 evokes cellular immunity that is cross-reactive with other members of the C species of RVs. Furthermore, when splenocytes from RV-C24 immunised mice were stimulated with VP0 peptide pools from strains of other RV-species, there was significant IFN-γ secretion to in response to all peptides from both RV-A and B species (FIG. 9C). This suggests that RV-C24 VP0 has the potential to induce cross-reactive cellular immunity against all species of RV.

To further demonstrate that immunisation of mice with RV-C24 VP0 induces immunity, the humoral response to the immunising antigen was assessed. Mice were immunised twice with RV-C24 VP0 protein with adjuvant and the serum subsequently harvested, as described above. The quantity of RV-C24 VP0 specific antibodies in the mouse serum was determined by ELISA. FIG. 10 is a set of line graphs that shows the levels of anti-RV-C24 VP0 antibodies in serum from mice following immunisation, as quantified by ELISA.

In all mice immunised with RV-C24 VP0 there are high levels of antibody that specifically recognises RV-C24 VP0, while in control immunised mice there are no discernible levels. These results demonstrate that, in addition to the induction of cross-reactive cellular immunity by immunisation with RV-C24 VP0, there is also a strong humoral response to the immunising protein.

### Recombinant RV-C24 VP0 Evoked Immunogenicity in Cynomolgus Monkeys

Cynomolgus monkeys were immunised twice with recombinant RV-C24 VP0 (rRV-C24 VP0) protein with adjuvant or adjuvant alone (control), and blood was taken 28 days after the first immunization and PBMCs prepared, as described. To quantify the level of cellular immunity evoked by immunisation with rRV-C24 VP0 protein, PBMCs were incubated with different peptide pool stimuli, corresponding to full-length VP0 from different strains of RV or controls, as indicated on the x axes in FIG. 11. PBMCs that are activated by the stimulus secrete the cytokine IFN-γ and the degree of IFN-γ secretion was quantified by ELISPOT analysis.

The results show that PBMCs from an rRV-C24 VP0 immunised representative cynomolgus monkey secrete IFN-γ in response to stimulation with VP0 peptide pools from a diverse set of A, B and C species RVs (FIG. 11B). PBMCs from an animal immunized with adjuvant alone (control) produced negligible IFN-γ in response to VP0 peptide pool stimulation (FIG. 11A).

When PBMCs from an rRV-C24 VP0 immunised cynomolgus monkey were stimulated with an irrelevant peptide control pool (FIG. 11B), there was negligible production of IFN-γ. When PBMCs from a control cynomolgus monkey were stimulated with an irrelevant peptide control pool or with the VP0 peptide pools (FIG. 11A), the production of IFN-γ was again negligible.

These results demonstrate that immunisation of cynomolgus monkeys with rRV-C24 VP0 evokes cellular immunity that is cross-reactive with strains from A, B and C species of RVs.

To demonstrate the Th phenotype of the cellular immune response, the IFN-γ responses of PBMCs from two cynomolgus monkeys immunized with adjuvant alone (control) or rRV-C24 VP0 protein, was compared to the IL-4 response by ELISPOT after stimulation with peptide pools. IFN-γ is a canonical Th1 cytokine and IL-4 is a canonical Th2 cytokine. FIG. 12A demonstrates that PBMCs from two cynomolgus monkeys immunized with rRV-C24 VP0 protein, but not adjuvant only (control), secrete IFN-γ in response to stimulation with peptide pools from C species of RV that are greater than that produced in response to the irrelevant peptide control pool, but that IL-4 secretion is negligible (FIG. 12B).

To further demonstrate that immunization with rRV-C24 VP0 induces immunity in cynomolgus monkeys, the humoral response to the immunising antigen was assessed. FIG. 13 is a histogram of RV-C24 VP0-binding IgG, as quantified by ELISA, in the plasma of cynomolgus monkeys at different timepoints after immunization with rRV-C24 VP0 protein. The results show that immunization with rRV-C24 VP0 evokes production of RV-C24 VP0-specific IgG antibodies, which increase in magnitude over time, whereas animals immunized with the adjuvant alone (control) did not produce detectable RV-C24 VP0-specific IgG.

Transfection of HEK292 Cells with RV VP0 mRNA

To demonstrate that RV VP0 mRNA is translated into full-length VP0 protein in cells, HEK293 cells were transfected with different RV VP0 mRNAs, as described above.

FIG. 14A is a Western blot of lysates from HEK293 cells transfected with RV-A16 VP0 mRNA made at 24, 48, 72 or 96 hours after transfection. A positive control of recombinant RV-A16 VP0 protein was loaded onto the gel (labelled rRV-A16 VP0). The results demonstrate that transfection of HEK293 cells with RV-A16 VP0 mRNA results in the expression of RV-A16 VP0 protein, which peaks at 48 hrs after transfection.

FIG. 14B is a Western blot of lysates from HEK293 cells transfected with RV-B06 or RV-C24 VP0 mRNA made at 24, 48, 72 or 96 hours after transfection. The results demonstrate that HEK293 cells transfected with RV-B06 VP0 or C24 VP0 mRNAs express RV-B06 and RV-C24 VP0 protein, respectively. Expression of both proteins peaks at 24 and 48 hours after transfection. By comparing the VP0 bands detected using the monoclonal antibody or antisera to the marker, it is observed that the VP0 bands are of the correct approximate molecular weight of 36 kDa.

RV VP0 mRNA Evoked Immunogenicity in Mice

Mice were immunised twice with RV-C24 VP0 mRNA, spleens harvested 42 days after the first immunization and splenocytes prepared, as described above. To quantify the level of cellular immunity evoked by immunisation with RV-C24 VP0 mRNA, splenocytes were incubated with peptide pools corresponding to full length VP0 proteins from different strains of RV or a control pool, as indicated in FIG. 15. Splenocytes that are activated by the stimulus secrete the cytokine IFN-γ and the degree of IFN-γ secretion was quantified by ELISPOT analysis.

The results show that splenocytes from RV-C24 VP0 mRNA immunised mice secrete IFN-γ in response to stimulation with VP0 peptide pools from a diverse set of C species RVs. When splenocytes from RV-C24 VP0 mRNA immunised mice were stimulated with an irrelevant peptide pool (FIG. 15) there was negligible production of IFN-γ. When the responses to the VP0 peptide pools stimuli were statistically compared to the responses to the irrelevant peptide pool, the results were significant. These results demonstrate that immunisation of mice with RV-C24 VP0 mRNA evokes cellular immunity that is cross-reactive with other members of C species RVs.

To further demonstrate that immunization with RV-C24 VP0 mRNA induces immunity in mice, the humoral response to the immunising antigen was assessed. FIG. 16 is a histogram showing the concentrations of RV-C24 VP0-binding IgG, as quantified by ELISA, in the serum of mice at different timepoints after immunization with RV-C24 VP0 mRNA. The results show that immunization with RV-C24 VP0 mRNA evokes production of RV-C24 VP0-specific IgG antibodies, which increase in magnitude over time, whereas control animals did not produce detectable RV-C24 VP0-specific IgG.

RV VP0 mRNA Evoked Immunogenicity in Cynomolgus Monkeys

FIG. 17 is a histogram that show the results from ELISPOT experiments to analyse the cellular immunity evoked by immunisation with an mRNA concatemer consisting of RV-C24/A16/B06 VP0 mRNAs in sequence separated by a 2A peptide-encoding sequence. Immunizing animals with the RV VP0 mRNA concatemer allows the immune responses to each antigen to be determined.

Cynomolgus monkeys were immunised twice with the RV-C24/A16/B06 VP0 mRNA concatemer or control, blood was taken 28 days after the first immunization and PBMCs prepared, as described above. To quantify the level of cellular immunity to each antigen evoked by immunisation with the RV-C24/A16/B06 VP0 mRNA concatemer, PBMCs were incubated with peptide pools corresponding to full-length RV-A16, B06, C24 VP0, or a control irrelevant peptide pool and IFN-γ (FIG. 17A) or IL-4 (FIG. 17B) secretion quantified by ELISPOT.

The results show that PBMCs from RV-C24/A16/B06 concatemer VP0 mRNA immunised cynomolgus monkeys secrete IFN-γ in response to stimulation with VP0 peptide pools corresponding to full-length RV-A16, B06 and C24 VP0. When PBMCs from RV-C24/A16/B06 concatemer VP0 mRNA immunised monkeys were stimulated with an irrelevant peptide pool there was negligible production of IFN-γ. PBMCs from control immunized animals produced negligible IFN-γ in response to stimulation with RV VP0 peptide pools or the irrelevant peptide control pool. The results in FIG. 17A demonstrate that immunisation of cynomolgus monkeys with RV-C24/A16/B06 concatemer VP0 mRNA evokes cellular immunity to each of the three antigens produced by the VP0 mRNA concatemer.

To demonstrate the Th phenotype of the cellular immune response, the IFN-γ response from PBMCs from cynomolgus monkeys immunized with RV-C24/A16/B06 VP0 mRNA concatemer was compared to the IL-4 response by ELISPOT. IFN-γ is a canonical Th1 cytokine and IL-4 is a canonical Th2 cytokine. FIG. 17B demonstrates that PBMCs from cynomolgus monkeys immunized with RV-C24/A16/B06 concatemer VP0 mRNA produce negligible IL-4 compared to IFN-γ (FIG. 17A) in response to stimulation with RV VP0 or control peptide pools. This result demonstrates that the cellular immune response primed by RV-C24/A16/B06 concatemer VP0 mRNA is Th1-polarized.

To further demonstrate that immunisation of cynomolgus monkeys with RV VP0 mRNA induces immunity, the humoral responses to each antigen produced by the RV-C24/A16/B06 VP0 mRNA concatemer was determined.

FIG. 18 is a histogram showing the concentrations of RV-A16 (FIG. 18A), RV-B06 (FIG. 18B) and RV-C24 (FIG. 18C) VP0-binding IgG, as quantified by ELISA, in the plasma of cynomolgus monkeys at different timepoints after immunization. These results show that immunization with the RV-C24/A16/B06 VP0 mRNA concatemer evokes production of RV-VP0-specific IgG antibodies to each of the three RV antigens in the concatemer in cynomolgus monkeys.

Efficacy of RV VP0 mRNA Immunization in Mice

To demonstrate that immunization with RV VP0 mRNA is protective against subsequent heterotypic RV infection, mice were immunized with RV-A16 VP0 mRNA and then infected intranasally with RV-A1, a heterotypic A species strain of RV to the immunizing strain. The methods are described above.

FIG. 19 is a histogram that shows the concentration of RV-A1 RNA in the lungs of control and RV-A16 VP0 mRNA immunized mice, as determined by RT-qPCR. The results demonstrate that while control and immunized mice have similar levels of viral RNA in the lungs one day after infection, by day six the immunized mice have significantly less viral RNA than control mice. By day 14, the concentration of viral RNA is negligible in both groups of animals. This result demonstrates that immunization with RV VP0 mRNA accelerates the clearance of a heterotypic RV infection from the lungs of mice.

RV VP0 mRNA Evoked Cellular Immunity

To demonstrate how immunization with RV VP0 mRNA protects mice against RV infection, the inventors harvested spleens from mice that had been immunized with RV-A16 VP0 mRNA or control and then infected with heterotypic RV-A1, at 14 days after infection and created splenocytes. The splenocytes were stimulated with peptide pools corresponding to full-length VP0 from a diverse set of strains covering each species of RV or a control irrelevant peptide pool. IFN-γ secretion in response to peptide stimulation was measured by ELISPOT.

The results shown in FIG. 20 were surprising and demonstrate that splenocytes from mice immunized with RV-A16 VP0 mRNA and then infected with RV-A1 secrete IFN-γ in response to stimulation with VP0 peptide pools from a wide range of RV strains, covering A, B and C species. When the results obtained with VP0 peptide pools were statistically compared to those obtained with the irrelevant peptide control pool, the results were highly significant.

In contrast, as shown in FIG. 21, in the control mice, there were negligible IFN-γ secreting splenocytes after stimulation with RV VP0 or control peptide pools. The results in FIGS. 20 and 21 demonstrate that immunization with VP0 mRNA from a single strain of RV produces surprising cross-strain cellular immunity.

To further demonstrate the mechanism by which RV VP0 mRNA immunization protects mice from infection with RV-A1, the inventors performed immunophenotyping on the lungs of RV-A16 VP0 mRNA immunized and non-immunized mice after infection with RV-A1. The methods are described above.

FIG. 22 is a histogram that shows the mean numbers of CD62⁻ CD44⁺ effector CD4⁺ (FIG. 22A) and CD8⁺ (FIG. 22B) T cells, respectively, in the lungs of RV-A16 VP0 mRNA immunized and control mice after infection with RV-A1. The results demonstrate that there are significantly more effector CD4⁺ and CD8⁺ T cells in the lungs of immunized mice 6 and 14 days after infection than in control mice.

RV VP0 mRNA Immunization Primes Th1-Polarized T Cell Immunity

To determine the Th phenotype primed by RV VP0 mRNA immunization, lungs were harvested from mice that had been infected with RV-A1 after immunization with RV-A16 VP0 mRNA or control. A single cell population was prepared from the lungs and the cells incubated with a peptide pool corresponding to full-length VP0 from RV-A1. Intracellular cytokine staining flow cytometry analysis was performed to measure the frequency of Th1 and Th2 cells, as described.

Th1 cells were identified as T-bet⁺ IFN-γ⁺ CD4⁺ cells and Th2 cells were identified as Gata3⁺ IL-4⁺ CD4⁺ cells. FIG. 23 is a set of correlation plots where the frequency of Th1 cells is plotted against the frequency of Th2 cells for each animal after stimulation of the lung cells with RV-A1VP0 peptide pool.

The results demonstrate that the frequency of RV-A1VP0 reactive Th1 cells is greater than RV-A1VP0 reactive Th2 cells in the lungs of immunized mice (FIG. 23B). In control mice (FIG. 23A), the opposite is observed, where the proportion of Th2 cells is greater than Th1 cells. This result demonstrates that RV VP0 mRNA primes a Th1-polarized T cell response, with a greater proportion of RV-reactive CD4⁺ T cells of the Th1 phenotype than the Th2 phenotype in the lungs of immunized mice.

### RV VP0 Primes Tissue Resident T Cell Memory

A critical component of protective cellular immunity elicited by natural infection with respiratory viruses is the formation of tissue resident memory T cells (T_{RM}) in the airway mucosa and lungs. These T_{RM} cells rapidly expand and clear subsequent infections.

To determine if an intramuscularly delivered RV VP0 mRNA vaccine is able to prime the formation of T_{RM} cells in the airways and lungs of immunized mice, bronchoalveolar lavage (BAL) and lungs were harvested from mice that had been infected with RV-A01 after immunization with RV-A16 VP0 mRNA or control. A single cell population was prepared from the lungs and immunophenotyping performed to identify and enumerate CD4⁺ and CD8⁺ T_{RM} cells at different post-infection timepoints. CD4⁺ and CD8⁺ T_{RM} cells were identified as CD103⁺ CD69⁺ CD4⁺ or CD103⁺ CD69⁺ CD8⁺ cells, respectively.

FIG. 24 is a set of histograms showing the number of CD4⁺ (FIG. 24A) and CD8⁺ (FIG. 24B) T_{RM} cells in the BAL of control and RV-A16 VP0 mRNA immunized mice at different post-RV-A1 infection timepoints. The results show that there are significantly more CD4⁺ and CD8⁺ T_{RM} cells in the BAL of immunized mice six days after infection compared to control mice.

FIG. 25 is a set of histograms showing the frequency of CD4⁺ (FIG. 25A) and CD8⁺ (FIG. 25B) T_{RM} cells in the BAL of control and RV-A16 VP0 mRNA immunized mice at different post-RV-A1 infection timepoints. The results show that CD4⁺ and CD8⁺ T_{RM} cells are present at a significantly higher proportion of total CD4⁺ or CD8⁺ cells, respectively, in the BAL of immunized mice six and fourteen days after infection compared to control mice.

FIG. 26 is a set of histograms showing the number of CD4⁺ (FIG. 26A) and CD8⁺ (FIG. 26B) T_{RM} cells in the lungs of control and RV-A16 VP0 mRNA immunized mice at different post-RV-A1 infection timepoints. The results show that there are significantly more CD4⁺ T_{RM} cells six days after infection and significantly more CD8⁺ T_{RM} cells six and fourteen days after infection in the lungs of immunized compared to control mice.

FIG. 27 is a set of histograms showing the frequency of CD4⁺ (FIG. 27A) and CD8⁺ (FIG. 27B) T_{RM} cells in the lungs of control and RV-A16 VP0 mRNA immunized mice at different post-RV-A1 infection timepoints. The results show that CD4⁺ and CD8⁺ T_{RM} cells are present at a significantly higher proportion of total CD4⁺ or CD8⁺ cells, respectively, in the lungs of immunized mice six and fourteen days after infection compared to control mice.

These results demonstrate that intramuscular administration of a RV VP0 mRNA vaccine primes T_{RM} cells in the airways and lungs of mice. Upon subsequent infection with a heterotypic strain of RV, the vaccine primed T_{RM} cells rapidly expand.

### CONCLUSIONS

The inventors have identified RV VP0 proteins from single representative strains, which are able to elicit broad cellular immune responses that cross-react with other RV strains from the same species. In particular, as illustrated in the Examples (FIGS. 3, 4, 5, 7, 8 and 9) splenocytes from RV-A16 immunised mice cross-react with VP0 peptides from other RV-A species strains, RV-B06 VP0 immunised mice cross react with VP0 peptides from other RV-B species members and peptides from some RV-A species strains, and splenocytes from either RV-C01, RV-C19 or RV-C24 VP0 immunised mice cross-react with peptides from other RV-C species members. Additionally, immunisation with VP0 protein from RV-C species confers cellular immunity that recognises VP0 peptides from both RV-A and RV-B species. Accordingly, an immunogenic composition according to the claimed invention, comprising VP0 peptides selected from RV-A16, RV-B06, RV-C01, RV-C19 and RV-C24, in any combination, can be used to immunise humans against infection with all RV strains.

Additionally, the inventors have demonstrated that immunisation with mRNA encoding the RV VP0 peptides, elicits a broad cellular immune response. For example, as shown in FIG. 15, the inventors demonstrated that immunisation of mice with RV-C24 VP0 mRNA evokes cellular immunity that is cross-reactive with other members of C species of RV.

Advantageously, therefore, the immunogenic composition according to the invention overcomes the issue of antigenic heterogeneity across RV strains, which has hampered RV vaccine development to date. Furthermore, the immunogenic composition is particularly effective, as vaccination with RV VP0 antigens will provide protection for patients who have chronic lung conditions and are therefore at risk of RV-induced exacerbation of their conditions, thereby reducing the associated morbidity, mortality and healthcare burden.

### REFERENCES

1. Gern, J. E. The ABCs of rhinoviruses, wheezing, and asthma. Journal of virology 84, 7418-7426 (2010).
2. Ritchie, A. I. et al. Pathogenesis of viral infection in exacerbations of airway disease. Annals of the American Thoracic Society 12, S115-S132 (2015).
3. Hewitt, R. et al. The role of viral infections in exacerbations of chronic obstructive pulmonary disease and asthma. Therapeutic advances in respiratory disease 10, 158-174 (2016).
4. Choi, T. et al. Enhanced neutralizing antibody responses to rhinovirus c and age-dependent patterns of infection. American Journal of Respiratory and Critical Care Medicine 203, 822-830 (2021).
5. Palmenberg, A. C. & Gern, J. E. Classification and evolution of human rhinoviruses. Methods in Molecular Biology 1221, (2015).
6. Glanville, N. et al. Cross-Serotype Immunity Induced by Immunization with a Conserved Rhinovirus Capsid Protein. PLOS Pathogens 9, e1003669 (2013).
7. Esquivel A, et al. Effects of Omalizumab on Rhinovirus Infections, Illnesses, and Exacerbations of Asthma. Am J Respir Crit Care Med. October 15; 196(8):985-992 (2017).

1-21. (canceled)

22. An immunogenic composition comprising an isolated human rhinovirus peptide, or an isolated polynucleotide encoding the peptide, wherein the peptide comprises an amino acid sequence as set out in SEQ ID No: 13, or a variant or fragment thereof having at least 80% sequence identity to SEQ ID No: 13, and/or wherein the peptide is encoded by the nucleotide sequence as set out in SEQ ID No: 14, or a variant or fragment thereof having at least 80% sequence identity to SEQ ID No: 14.

23. The immunogenic composition according to claim 22, wherein the polynucleotide comprises a nucleic acid sequence encoding the peptide, optionally wherein the nucleic acid sequence is placed under the control of the elements necessary for its expression in a mammalian cell.

24. The immunogenic composition according to claim 23, wherein the nucleic acid is DNA or RNA, optionally wherein the RNA is messenger RNA (mRNA) or self-amplifying RNA (saRNA).

25. The immunogenic composition according to claim 22, wherein the immunogenic composition further comprises an adjuvant.

26. The immunogenic composition according to claim 22, wherein the immunogenic composition is a vaccine.

27. A method of eliciting an immune response in a subject against a rhinovirus infection, the method comprising administering, to a subject in need thereof, a therapeutically effective amount of the immunogenic composition according to claim 22.

28. The method according to claim 27, wherein the immunogenic composition elicits an immune response against at least one, at least two, or at least three species of rhinoviruses, more particularly, against RV-A, RV-B, and/or RV-C.

29. The method according to claim 27, wherein the immunogenic composition elicits an immune response against at least one strain of RV-A, at least one strain of RV-B, and/or at least one strain of RV-C.

30. The method according to claim 27, wherein the immunogenic composition elicits an immune response against common colds, virus-induced wheezing illnesses, exacerbations of asthma, bronchiectasis, chronic obstructive pulmonary disease (COPD), cystic fibrosis and/or chronic fibrosing lung disease.


SEQUENCE LISTING (27 sequences)

SEQ ID NO: 1 - protein, 330 aa, Rhinovirus A
MGAQVSRQNVGTHSTQNMVSNGSSLNYFNINYFKDAASSGASRLDFSQDPSKFTDPVKDVLEKGIPTLQSPSVEACGYSDRIIQITRGDSTITSQDVANAVVGYGVWPHYLTPQDATAIDKPTQPDTSSNRFYTLDSKMWNSTSKGWWWKLPDALKDMGIFGENMFYHFLGRSGYTVHVQCNASKFHQGTLLVVMIPEHQLATVNKGNVNAGYKYTHPGEAGREVGTQVENEKQPSDDNWLNFDGTLLGNLLIFPHQFINLRSNNSATLIVPYVNAVPMDSMVRHNNWSLVIIPVCQLQSNNISNIVPITVSISPMCAEFSGARAKTVVQ

SEQ ID NO: 2 - DNA, 990 bp, Rhinovirus A
  source 1..990: mol_type = genomic DNA
atgggtgcgcaggtgagccgtcagaacgtgggcacccatagcacccagaacatggtgagcaacggcagcagcctgaactatttcaacatcaactatttcaaagatgcggcgagcagcggtgcgagccgtctggatttcagccaggatccgagcaaatttaccgatccggtgaaagatgtgctggaaaaaggcattccgaccctgcagagcccgagcgtggaagcgtgcggctatagcgatcgtattattcagattacccgtggcgatagcaccattaccagccaggatgtggcgaacgcggtggtgggctatggtgtgtggccgcattatctgaccccgcaggatgcgaccgcgattgataaaccgacccagccggataccagcagcaaccgtttttataccctggatagcaaaatgtggaacagcaccagcaaaggttggtggtggaaactgccggatgcgctgaaagatatgggcattttcggcgaaaacatgttctatcattttctgggccgtagcggctataccgtgcatgtgcagtgcaacgcgagcaaatttcatcagggcaccctgctggttgtgatgattccggaacatcagctggccaccgtgaacaaaggcaacgtgaacgcgggctataaatatacccatccgggcgaagcgggtcgtgaagtgggcacccaggtggaaaacgaaaaacagccgagcgatgataactggctgaactttgatggcaccctgctgggcaacctgctgatttttccgcatcagttcattaacctgcgtagcaacaacagcgcgaccctgattgtgccgtatgtgaacgcggtgccgatggatagcatggtgcgtcataacaattggagcctggtgattattccggtgtgccagctgcagagcaacaacattagcaacattgtgccgattaccgtgagcattagcccgatgtgcgcggaatttagcggtgcgcgtgcgaaaaccgtggtgcag

SEQ ID NO: 3 - RNA, 990 nt, Rhinovirus A
  source 1..990: mol_type = genomic RNA
atgggtgcgcaggtgagccgtcagaacgtgggcacccatagcacccagaacatggtgagcaacggcagcagcctgaactatttcaacatcaactatttcaaagatgcggcgagcagcggtgcgagccgtctggatttcagccaggatccgagcaaatttaccgatccggtgaaagatgtgctggaaaaaggcattccgaccctgcagagcccgagcgtggaagcgtgcggctatagcgatcgtattattcagattacccgtggcgatagcaccattaccagccaggatgtggcgaacgcggtggtgggctatggtgtgtggccgcattatctgaccccgcaggatgcgaccgcgattgataaaccgacccagccggataccagcagcaaccgtttttataccctggatagcaaaatgtggaacagcaccagcaaaggttggtggtggaaactgccggatgcgctgaaagatatgggcattttcggcgaaaacatgttctatcattttctgggccgtagcggctataccgtgcatgtgcagtgcaacgcgagcaaatttcatcagggcaccctgctggttgtgatgattccggaacatcagctggccaccgtgaacaaaggcaacgtgaacgcgggctataaatatacccatccgggcgaagcgggtcgtgaagtgggcacccaggtggaaaacgaaaaacagccgagcgatgataactggctgaactttgatggcaccctgctgggcaacctgctgatttttccgcatcagttcattaacctgcgtagcaacaacagcgcgaccctgattgtgccgtatgtgaacgcggtgccgatggatagcatggtgcgtcataacaattggagcctggtgattattccggtgtgccagctgcagagcaacaacattagcaacattgtgccgattaccgtgagcattagcccgatgtgcgcggaatttagcggtgcgcgtgcgaaaaccgtggtgcag

SEQ ID NO: 4 - protein, 331 aa, Rhinovirus B
MGAQVSTQKSGSHENQNILTNGSNQTFTVINYYKDAASSSSAGQSFSTDPSKFTEPVKDLMLKGAPALNSPNVEACGYSDRVQQITLGNSTITTQEAANAVVCYAEWPEYLPDGDASDVNKTSKPDTSVCRFYTLESKTWQNSSKGWCWKLPDALKDMGVFGQNMFFHSLGRSGYTIHVQCNATKFHSGCLLVVVIPEHQLASHKGGNVSVKYKYTHPGESGIDLSTQLDEDGPVKDPVYSMNGTLLGNLLIFPHQFINLRTNNTATIVVPYINSVPIDSMTRHNNVSLLVIPIAPLVAPTGSPQTLPVTVTIAPMCTEFTGIRSKTIVPQ

SEQ ID NO: 5 - DNA, 993 bp, Rhinovirus B
  source 1..993: mol_type = genomic DNA
atgggcgctcaagtttcaacacaaaagagtggttctcacgagaatcagaacatcttaactaatggctcaaatcagacgttcacagttataaattattataaagatgcagctagctcatcatcagcaggtcagtctttctcaacggacccatcaaaattcacagaaccagttaaagacctgatgttgaaaggtgccccagcattgaactcaccaaatgttgaagcatgtggttatagtgacagagtccaacaaattactctaggtaattccactataacaacccaggaagcagccaatgcggttgtgtgttatgctgagtggcctgaatacttgccagatggtgatgctagtgatgttaataagacttccaaaccagacacatctgtatgtagattctacacattggagagtaaaacatggcaaaacagctcaaagggatggtgttggaaactaccagatgctctcaaggatatgggtgtgtttgggcaaaacatgttcttccactcgcttggtagatctggctatactatacatgtgcaatgtaatgcaactaaattccatagtggctgtcttcttgttgttgttatcccagaacaccaattggcatctcacaaaggcgggaatgtgtcagtgaagtataaatatacacatccaggagagagtggtatagacttgagtacacagttagatgaggatggtccagtgaaagatcctgtctatagcatgaatggcactctattaggtaacttactcatcttccctcaccagtttatcaacctcagaacaaacaatacagccacaatagttgtgccatatattaattcagtgccaatagactcaatgacacgccataataacgtgtcactcttagtgattcccatagcacctctagtggctccaactggatcaccacaaacacttccagtgacagtgactatagctcccatgtgtacagagttcacgggtattagatcaaaaaccattgttccacag

SEQ ID NO: 6 - RNA, 993 nt, Rhinovirus B
  source 1..993: mol_type = genomic RNA
atgggcgctcaagtttcaacacaaaagagtggttctcacgagaatcagaacatcttaactaatggctcaaatcagacgttcacagttataaattattataaagatgcagctagctcatcatcagcaggtcagtctttctcaacggacccatcaaaattcacagaaccagttaaagacctgatgttgaaaggtgccccagcattgaactcaccaaatgttgaagcatgtggttatagtgacagagtccaacaaattactctaggtaattccactataacaacccaggaagcagccaatgcggttgtgtgttatgctgagtggcctgaatacttgccagatggtgatgctagtgatgttaataagacttccaaaccagacacatctgtatgtagattctacacattggagagtaaaacatggcaaaacagctcaaagggatggtgttggaaactaccagatgctctcaaggatatgggtgtgtttgggcaaaacatgttcttccactcgcttggtagatctggctatactatacatgtgcaatgtaatgcaactaaattccatagtggctgtcttcttgttgttgttatcccagaacaccaattggcatctcacaaaggcgggaatgtgtcagtgaagtataaatatacacatccaggagagagtggtatagacttgagtacacagttagatgaggatggtccagtgaaagatcctgtctatagcatgaatggcactctattaggtaacttactcatcttccctcaccagtttatcaacctcagaacaaacaatacagccacaatagttgtgccatatattaattcagtgccaatagactcaatgacacgccataataacgtgtcactcttagtgattcccatagcacctctagtggctccaactggatcaccacaaacacttccagtgacagtgactatagctcccatgtgtacagagttcacgggtattagatcaaaaaccattgttccacag

SEQ ID NO: 7 - protein, 330 aa, Rhinovirus C
MGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVRWGGSSKGWWWKLPDCLKNMGIFGQNMYYHAMGRSGYIIHTQCNATKFHSGCLLVAVVPEHQLAYIGGTNAQVSYKHTHPGERGHEIGLNTSRGDNRPDEDPFFNCNGTLLGNLTIFPHQLINLRTNNSSTIVVPYINCTPMDSMLRHNNVSLVIIPICPLRTPSGAPTTLPITVSIAPDRSEFSGARQSATRQ

SEQ ID NO: 8 - DNA, 990 bp, Rhinovirus C
  source 1..990: mol_type = genomic DNA
atgggcgctcaggtttccaagcagagtgtgggagctcatgaaactatggtgcatgctggctctggtgcagttgttaaatacttcaacatcaattattacaaggatgctgctagctccggtttgaccaaacaagatttctcacaagacccatccaagtttactcagcctgtagcagatttactgacaaatccagctttgatgtccccatcagttgaggcgtgtggatactccgataggctcaagcaaatcactatcggaagctccaccattacaacacaagattctgtcaacaccatagttgcctatggcgagtggcctagttatttgtcagatttagatgcatcatctgttgataaacccacacatcctgaaacatcggctgacagattctacactttggacagcgtacgctggggtggtagttccaaaggatggtggtggaagttgccagactgtttgaagaacatgggtatatttgggcagaacatgtactaccacgctatgggaaggtctggttacatcatacacacccagtgcaatgccactaagttccacagtggatgtctgttggtagcggttgttcccgaacaccagctagcttacattgggggtaccaatgcacaagttagctacaaacatactcacccaggtgaacgtgggcatgaaataggtctcaacacaagcagaggtgacaaccggccagatgaggatcccttttttaattgcaacggaacactattaggtaacttgactattttccctcaccagctcataaacctgaggacaaataactccagcaccatagttgtgccttacattaactgcacaccaatggacagtatgttacgccacaacaatgtctctcttgtaattatacccatctgtcctttacgaactcccagtggagcacccactaccttaccaattactgtttcaattgccccagacagatctgaattctctggcgccaggcagtccgccacccgccag

SEQ ID NO: 9 - RNA, 990 nt, Rhinovirus C
  source 1..990: mol_type = genomic RNA
atgggcgctcaggtttccaagcagagtgtgggagctcatgaaactatggtgcatgctggctctggtgcagttgttaaatacttcaacatcaattattacaaggatgctgctagctccggtttgaccaaacaagatttctcacaagacccatccaagtttactcagcctgtagcagatttactgacaaatccagctttgatgtccccatcagttgaggcgtgtggatactccgataggctcaagcaaatcactatcggaagctccaccattacaacacaagattctgtcaacaccatagttgcctatggcgagtggcctagttatttgtcagatttagatgcatcatctgttgataaacccacacatcctgaaacatcggctgacagattctacactttggacagcgtacgctggggtggtagttccaaaggatggtggtggaagttgccagactgtttgaagaacatgggtatatttgggcagaacatgtactaccacgctatgggaaggtctggttacatcatacacacccagtgcaatgccactaagttccacagtggatgtctgttggtagcggttgttcccgaacaccagctagcttacattgggggtaccaatgcacaagttagctacaaacatactcacccaggtgaacgtgggcatgaaataggtctcaacacaagcagaggtgacaaccggccagatgaggatcccttttttaattgcaacggaacactattaggtaacttgactattttccctcaccagctcataaacctgaggacaaataactccagcaccatagttgtgccttacattaactgcacaccaatggacagtatgttacgccacaacaatgtctctcttgtaattatacccatctgtcctttacgaactcccagtggagcacccactaccttaccaattactgtttcaattgccccagacagatctgaattctctggcgccaggcagtccgccacccgccag

SEQ ID NO: 10 - protein, 330 aa, Rhinovirus C
MGAQVSKQNVGSHENSVSASNGSVIKYFNINYYKDSASSGLTKQDFSQDPSKFTQPLVDTLTNPALMSPSVEACGFSDRLKQITIGNSTITTQDSLNTVLAYGEWPSYLSDLDATSVDKPSHPETSSDRFYTLESVDWKSGSKGWWWKFPDALKDMGMFGQNMYHHSMGRFGALIHVQCNATKFHSGCLLIMVVPEHQLAYIGAEGVKVRYEHTHPGEKGHTLKGSQDRSTNNPDDNPFYMCNGTLLGNGMLYPHQMINLRTNNSATIVIPYINCVPMDNMLRHNNFSLVIVPIVPLRPGNSGAPILPITVTIAPYKSEFSGAMKSQPNQ

SEQ ID NO: 11 - DNA, 990 bp, Rhinovirus C
  source 1..990: mol_type = genomic DNA
atgggtgcacaggtcagcaaacagaatgttggatcacacgaaaactctgtgtctgctagtaatggctctgtaatcaagtactttaacatcaattactataaagactctgccagctctgggttgactaaacaagacttttcacaagacccgtccaaatttactcaaccactcgttgacacactgacaaatcccgccttgatgtccccaagtgttgaagcttgtgggttttccgataggcttaaacaaatcactatcggaaactccactattacaacacaagattcgctcaacaccgtcttggcgtatggagaatggccttcatacctctctgacctagatgccacctcagtagataaaccatcacacccagaaacctcctcagaccgattctatacactagaaagtgttgattggaagtccggatctaaaggttggtggtggaaatttccagatgccctaaaagatatgggaatgtttgggcaaaacatgtaccaccattcaatgggtaggtttggagctctcatacatgtccaatgtaatgctactaaatttcacagtggttgtctcctgatcatggttgtccccgaacatcaattagcatatataggggcagaaggtgtcaaggttagatatgagcacacacacccaggagaaaagggccataccttgaaaggtagccaggatagatcaactaacaatccagatgataacccattctacatgtgtaacggtacactactgggtaatggtatgttatatcctcatcagatgatcaacctccgcaccaacaactcagccactattgtaattccatacatcaactgtgtacccatggacaatatgttgcgccacaacaacttcagtctggtgattgtaccaatagtacccttgagacctgggaactcaggagctcctatcttacccatcactgtgaccatagcaccatacaaatcagagttttctggagctatgaaatcccaacccaatcag

SEQ ID NO: 12 - RNA, 993 nt, Rhinovirus C
  source 1..993: mol_type = genomic RNA
atgggtgcacaggtcagcaaacagaatgttggatcacacgaaaactctgtgtctgctagtaatggctctgtaatcaagtactttaacatcaattactataaagactctgccagctctgggttgactaaacaagacttttcacaagacccgtccaaatttactcaaccactcgttgacacactgacaaatcccgccttgatgtccccaagtgttgaagcttgtgggttttccgataggcttaaacaaatcactatcggaaactccactattacaacacaagattcgctcaacaccgtcttggcgtatggagaatggccttcatacctctctgacctagatgccacctcagtagataaaccatcacacccagaaacctcctcagaccgattctatacactagaaagtgttgattggaagtccggatctaaaggttggtggtggaaatttccagatgccctaaaagatatgggaatgtttgggcaaaacatgtaccaccattcaatgggtaggtttggagctctcatacatgtccaatgtaatgctactaaatttcacagtggttgtctcctgatcatggttgtccccgaacatcaattagcatatataggggcagaaggtgtcaaggttagatatgagcacacacacccaggagaaaagggccataccttgaaaggtagccaggatagatcaactaacaatccagatgataacccattctacatgtgtaacggtacactactgggtaatggtatgttatatcctcatcagatgatcaacctccgcaccaacaactcagccactattgtaattccatacatcaactgtgtacccatggacaatatgttgcgccacaacaacttcagtctggtgattgtaccaatagtacccttgagacctgggaactcaggagctcctatcttacccatcactgtgaccatagcaccatacaaatcagagttttctggagctatgaaatcccaacccaattaacag

SEQ ID NO: 13 - protein, 328 aa, Rhinovirus C
MGAQVSKQNVGSHENSVSATGGSVIKYFNINYYKDSASSGLTKQDFSQDPSKFTQPLAEALTNPALMSPTVEACGMSDRLKQITIGNSTITTQDTLNSILAYGEWPKYLSDLDASSVDKPTHPETSSDRFYTLTSVDWTTTSKGWWWKLPDCLKDMGIFGQNLYHHALGRSGYIIHTQCNATKFNSGCLIVAVVPEHQLAYIGEANVNVGYDHTHPGEGGHVIGSNVRRDNKQPDEDPFFNCNGTLLGNITIFPHQLINLRTNNSSTIVVPYINCVPMDNMLRHNNLSLVIIPIVPLRAANGVTKVPITISIAPDKSEFSGARQSVKQ

SEQ ID NO: 14 - DNA, 984 bp, Rhinovirus C
  source 1..984: mol_type = genomic DNA
atgggcgcacaggtcagcaagcaaaatgtcggctcgcatgaaaactcagtctcagccacaggtggatccgtgattaagtatttcaacatcaattactacaaggattctgctagctctggcttgactaaacaagatttttcccaagacccatcgaaattcacacaacctctagcagaagcacttacaaatccagctttaatgtcaccaactgttgaagcatgtgggatgtccgataggcttaaacaaattactatcgggaattccactataacaacacaagatacactaaactctatactggcatatggggagtggcccaaatacttgagtgacctggacgcttcctcagtggataaacctacccacccagaaacatcatctgatagattttacacattaactagtgtagattggaccactacgtctaaaggttggtggtggaagttgcctgattgccttaaagatatgggcatcttcgggcaaaatctgtaccatcatgcattgggtaggtcagggtacataatacacacccaatgtaatgccacaaaattcaatagtggttgtctaatagtggctgttgtaccagaacaccagctagcttacataggtgaagcaaatgtcaatgttggttatgatcacacacaccctggtgagggaggacatgtaattggttcaaatgttaggagagataacaagcaacctgatgaagaccccttctttaattgtaatgggaccctgcttggtaacatcactatattcccacaccagctcataaacttgaggacaaacaattccagcacaattgttgtaccatacattaattgtgtacctatggacaacatgctcaggcacaacaacctatctctagttattattccaatcgttcctctcagagccgcaaatggtgtcaccaaggtccccattacaatctcaatagcaccagataagtcagagttctcaggggctagacagtctgtaaaacag

SEQ ID NO: 15 - RNA, 987 nt, Rhinovirus C
  source 1..987: mol_type = genomic RNA
atgggcgcacaggtcagcaagcaaaatgtcggctcgcatgaaaactcagtctcagccacaggtggatccgtgattaagtatttcaacatcaattactacaaggattctgctagctctggcttgactaaacaagatttttcccaagacccatcgaaattcacacaacctctagcagaagcacttacaaatccagctttaatgtcaccaactgttgaagcatgtgggatgtccgataggcttaaacaaattactatcgggaattccactataacaacacaagatacactaaactctatactggcatatggggagtggcccaaatacttgagtgacctggacgcttcctcagtggataaacctacccacccagaaacatcatctgatagattttacacattaactagtgtagattggaccactacgtctaaaggttggtggtggaagttgcctgattgccttaaagatatgggcatcttcgggcaaaatctgtaccatcatgcattgggtaggtcagggtacataatacacacccaatgtaatgccacaaaattcaatagtggttgtctaatagtggctgttgtaccagaacaccagctagcttacataggtgaagcaaatgtcaatgttggttatgatcacacacaccctggtgagggaggacatgtaattggttcaaatgttaggagagataacaagcaacctgatgaagaccccttctttaattgtaatgggaccctgcttggtaacatcactatattcccacaccagctcataaacttgaggacaaacaattccagcacaattgttgtaccatacattaattgtgtacctatggacaacatgctcaggcacaacaacctatctctagttattattccaatcgttcctctcagagccgcaaatggtgtcaccaaggtccccattacaatctcaatagcaccagataagtcagagttctcaggggctagacagtctgtaaaacagcag

SEQ ID NO: 16 - protein, 328 aa, Rhinovirus C
MGAQVSRQSVGSHETMIHAGTGAVVKYFNVNYYKDAASSGLTKQDFSQDPSKFTQPVADILTNPALMSPSVEACGFSDRLKQITIGNSTITTQDAVNTIVAYGEWPSYLSDLDATSVDKPTHPETSSDRFYTLRSVDWRGGSKGWWWKLPDCLKDMGIFGQNMFHHAMGRSGYIIHTQCNATKFHSGCLLVAVVPEHQLAYIGADARVSYEHTHPGERGHTIGSNADRNNHQPDENPFFNCNGTLLGNLTIFPHQLINLRTNNSSTIVVPYINCTPMDSMLRHNNVSLVIIPICPLRPPTTGQQVLPITISIAPIKSEFSGARQAIKQ

SEQ ID NO: 17 - DNA, 984 bp, Rhinovirus C
  source 1..984: mol_type = genomic DNA
atgggcgcacaggtgtctagacagagtgtcgggagccatgaaaccatgatccatgctgggactggagccgtagtaaagtacttcaatgtcaattattacaaagatgcagcaagctctggattgaccaagcaagatttctcccaggatccatccaaatttactcaacccgtggccgacatcctgaccaacccagctttgatgtccccctccgttgaagcttgtgggttttccgataggctcaagcaaatcactattggaaactccactataacaacccaagatgcagtcaatacaattgtggcatacggggaatggcccagttatctatcagatttggatgcgacttcagttgataaaccgacgcaccctgagacatcatcagacagattttacacgctgaggagtgttgactggcgaggtggttcaaaagggtggtggtggaaactgcctgattgtctgaaagacatgggaatatttgggcaaaacatgttccaccatgccatgggaagatcaggatacatcatacacacccagtgcaatgcaactaaatttcacagtggatgtttactcgtggcagtagtgcctgaacaccaattagcatacataggtgcagatgcacgagtgagttatgagcacacccaccctggtgagcgaggccatacaattggtagcaacgcagacaggaataaccatcagccagatgaaaatcctttcttcaactgtaacgggactttacttggaaatttgactatcttcccacaccagcttataaatttaaggacaaacaactcaagcaccatcgtcgtgccttacatcaattgcactcctatggatagcatgttacggcacaacaatgtatcacttgttatcatacccatttgccctttacggcccccaaccacagggcagcaagtcttaccgatcaccatttctattgctcccataaaatcagagttttcaggggctaggcaggccattaaacaa

SEQ ID NO: 18 - RNA, 984 nt, Rhinovirus C
  source 1..984: mol_type = genomic RNA
atgggcgcacaggtgtctagacagagtgtcgggagccatgaaaccatgatccatgctgggactggagccgtagtaaagtacttcaatgtcaattattacaaagatgcagcaagctctggattgaccaagcaagatttctcccaggatccatccaaatttactcaacccgtggccgacatcctgaccaacccagctttgatgtccccctccgttgaagcttgtgggttttccgataggctcaagcaaatcactattggaaactccactataacaacccaagatgcagtcaatacaattgtggcatacggggaatggcccagttatctatcagatttggatgcgacttcagttgataaaccgacgcaccctgagacatcatcagacagattttacacgctgaggagtgttgactggcgaggtggttcaaaagggtggtggtggaaactgcctgattgtctgaaagacatgggaatatttgggcaaaacatgttccaccatgccatgggaagatcaggatacatcatacacacccagtgcaatgcaactaaatttcacagtggatgtttactcgtggcagtagtgcctgaacaccaattagcatacataggtgcagatgcacgagtgagttatgagcacacccaccctggtgagcgaggccatacaattggtagcaacgcagacaggaataaccatcagccagatgaaaatcctttcttcaactgtaacgggactttacttggaaatttgactatcttcccacaccagcttataaatttaaggacaaacaactcaagcaccatcgtcgtgccttacatcaattgcactcctatggatagcatgttacggcacaacaatgtatcacttgttatcatacccatttgccctttacggcccccaaccacagggcagcaagtcttaccgatcaccatttctattgctcccataaaatcagagttttcaggggctaggcaggccattaaacaa

SEQ ID NO: 19 - protein, 332 aa, Rhinovirus A
MGAQVSRQNVGTHTTQNAVSNGSSLNYFNINYFKDAASSGASRLDFSQDPSKFTDPVKDVLTKGIPTLQSPSVEACGYSDRIIQITRGNSTITSQDIANAVVAYGVWPEYLPSQDASAIDKPTHPDTSSNRFYTLESKNWTATSKGWWWKLPDALKNMGIFGENLFYHFLGRAGYTVHVQCNASKFHQGTLIVAAIPEHQLAFISKGNVTVGYNYTHPGEQGREIGTFGPSSTLDRQPSDENWLNCDGTLLGNITVYPHQYINLRSNNSATLILPYVNAVPMDSMVRHNNWSIVIIPISKLETEGGTTIVPITLSISPMFSEFSGARNNVPQ

SEQ ID NO: 20 - DNA, 996 bp, Rhinovirus A
  source 1..996: mol_type = genomic DNA
atgggcgctcaagtatctaggcagaatgttggcacacacaccacacaaaatgctgtatcaaatggctctagtttgaattattttaacatcaactactttaaagatgctgcctcaagtggagcatcacgactagatttttcacaagatcctagtaaattcactgatccagttaaagatgttcttacaaaaggtataccaactctacaatcacccagtgttgaggcatgtggttattcagataggatcatacaaataacacgagggaattcaacaattacttcacaagatatagcaaatgctgtagtagcctatggagtttggcctgaatatctgcctagtcaagatgctagtgcaatagacaaaccaacacatcctgacacctcttctaacagattctatacattggaaagcaaaaattggactgcaacctcaaaaggatggtggtggaaacttccagatgctctcaagaatatggggatctttggtgagaatcttttctatcatttccttggtagagccgggtacactgtacatgtgcagtgtaatgctagcaagtttcaccagggtaccctaatagttgctgccattccagaacatcagttggctttcatttccaagggtaatgtcacagttggttataattatacacaccctggtgaacaaggccgtgaaataggcacctttggacccagtagcacattggacagacaacctagtgatgaaaattggcttaattgtgatggcacacttttgggaaatataacagtgtatccacaccaatatatcaacctcagaagtaacaattcagcaacattaatattaccatatgttaacgccgtacccatggactctatggttaggcataacaattggagtatagtgattatacccatatctaaattagagactgagggtgggacaacaattgtacccataactttatctattagcccaatgttttcagagttttctggggcgagaaacaatgtccctcaa

SEQ ID NO: 21 - RNA, 996 nt, Rhinovirus A
  source 1..996: mol_type = genomic RNA
atgggcgctcaagtatctaggcagaatgttggcacacacaccacacaaaatgctgtatcaaatggctctagtttgaattattttaacatcaactactttaaagatgctgcctcaagtggagcatcacgactagatttttcacaagatcctagtaaattcactgatccagttaaagatgttcttacaaaaggtataccaactctacaatcacccagtgttgaggcatgtggttattcagataggatcatacaaataacacgagggaattcaacaattacttcacaagatatagcaaatgctgtagtagcctatggagtttggcctgaatatctgcctagtcaagatgctagtgcaatagacaaaccaacacatcctgacacctcttctaacagattctatacattggaaagcaaaaattggactgcaacctcaaaaggatggtggtggaaacttccagatgctctcaagaatatggggatctttggtgagaatcttttctatcatttccttggtagagccgggtacactgtacatgtgcagtgtaatgctagcaagtttcaccagggtaccctaatagttgctgccattccagaacatcagttggctttcatttccaagggtaatgtcacagttggttataattatacacaccctggtgaacaaggccgtgaaataggcacctttggacccagtagcacattggacagacaacctagtgatgaaaattggcttaattgtgatggcacacttttgggaaatataacagtgtatccacaccaatatatcaacctcagaagtaacaattcagcaacattaatattaccatatgttaacgccgtacccatggactctatggttaggcataacaattggagtatagtgattatacccatatctaaattagagactgagggtgggacaacaattgtacccataactttatctattagcccaatgttttcagagttttctggggcgagaaacaatgtccctcaa

SEQ ID NO: 22 - protein, 335 aa, Rhinovirus A
MGAQVSRQNVGTHSTQNSVSNGSSLNYFNINYFKDAASSGASKLEFSQDPSKFTDPVKDVLEKGIPTLQSPTVEACGYSDRIMQITRGDSTITSQDVANAVIGYGVWPHYLSAEDATAIDKPTQPDTSSNRFYTLESKTWNRQSKGWWWKLPDALKDMGIFGENMYYHYLGRSGYTVHVQCNASKFHQGTLLVVMIPEHQLASVGTDKVGPGYAFTHPGEAGRQIGNVSEPTSKHPSDDNWLNFDGTLLGNVLIFPHQFINLRSNNSATIIVPYVNAVPMDSMLRHNNWSLVIIPISELQAENATNVTVPITVSISPMFAEFSGARARPARATTQ

SEQ ID NO: 23 - DNA, 1005 bp, Rhinovirus A
  source 1..1005: mol_type = genomic DNA
atgggcgctcaagtatccagacaaaatgttggaactcattctacccaaaattcagtttcaaatggatcgagtttaaattattttaacataaattactttaaggatgctgcatcaagtggagcctcaaaacttgaattttctcaagatcctagcaaattcactgacccggtgaaagatgttttagaaaaaggtattccaacactacaatcccctacggtggaggcatgtggttattcagatcggataatgcaaattacaagaggtgactccacaataacatctcaagatgtggctaatgcagttataggttatggagtctggccacactatctgagtgctgaagatgctactgctattgataaacctacccagccagacacctcatcaaatagattttacaccttggagagtaagacatggaataggcaatccaaaggatggtggtggaaattgccagatgcacttaaagatatgggcatatttggagaaaatatgtattatcattaccttggcaggagtggttatacagtacatgtccaatgcaatgctagtaagtttcatcaaggaacattactggtagtgatgatacctgagcaccaactggcaagtgtaggtactgataaagtcggacctggatatgctttcacgcatcccggtgaggcaggtagacagattggcaatgtgagtgaaccgacttcaaagcatccaagtgatgacaattggctcaactttgatggcaccttactgggtaatgttcttatattcccacaccaatttatcaatttaaggagcaataattctgcaacaataatagttccttatgtgaatgctgtcccaatggattcaatgttaagacataataattggagtttagtgataattccaatcagtgaattacaagcagagaatgccactaatgtcacggtccctattactgtatcaattagtccaatgtttgctgaattttctggagcacgtgcaaggccagcaagagctactacacag

SEQ ID NO: 24 - RNA, 1005 nt, Rhinovirus A
  source 1..1005: mol_type = genomic RNA
atgggcgctcaagtatccagacaaaatgttggaactcattctacccaaaattcagtttcaaatggatcgagtttaaattattttaacataaattactttaaggatgctgcatcaagtggagcctcaaaacttgaattttctcaagatcctagcaaattcactgacccggtgaaagatgttttagaaaaaggtattccaacactacaatcccctacggtggaggcatgtggttattcagatcggataatgcaaattacaagaggtgactccacaataacatctcaagatgtggctaatgcagttataggttatggagtctggccacactatctgagtgctgaagatgctactgctattgataaacctacccagccagacacctcatcaaatagattttacaccttggagagtaagacatggaataggcaatccaaaggatggtggtggaaattgccagatgcacttaaagatatgggcatatttggagaaaatatgtattatcattaccttggcaggagtggttatacagtacatgtccaatgcaatgctagtaagtttcatcaaggaacattactggtagtgatgatacctgagcaccaactggcaagtgtaggtactgataaagtcggacctggatatgctttcacgcatcccggtgaggcaggtagacagattggcaatgtgagtgaaccgacttcaaagcatccaagtgatgacaattggctcaactttgatggcaccttactgggtaatgttcttatattcccacaccaatttatcaatttaaggagcaataattctgcaacaataatagttccttatgtgaatgctgtcccaatggattcaatgttaagacataataattggagtttagtgataattccaatcagtgaattacaagcagagaatgccactaatgtcacggtccctattactgtatcaattagtccaatgtttgctgaattttctggagcacgtgcaaggccagcaagagctactacacag

SEQ ID NO: 25 - protein, 336 aa, Rhinovirus A
MGAQVSRQNVGTHSTQNSVSNGSSLNYFNINYFKDAASSGASRLDFSQDPSKFTDPVKDVLEKGIPTLQSPTVEACGYSDRLIQITRGDSTITSQDIANAVVAYGVWPSYLTPDDATAIDKPTQPDTSSNRFYTLDSRSWTSASSGWWWKLPDALKNMGIFGENMFYHFLGRSGYTIHVQCNSSKFHQGLLIVAAIPEHQLASATSGNVSVGYNHTHPGEQGREVVPSRTSSDNKRPSDDSWLNFDGTLLGNLPIYPHQYINLRTNNSATLILPYVNAVPMDSMLRHNNWSLVIIPICPLQVQPGGTQSIPITVSISPMFSEFSGPRNKVVFSTTQ

SEQ ID NO: 26 - DNA, 1008 bp, Rhinovirus A
  source 1..1008: mol_type = genomic DNA
atgggtgcacaagtatctagacagaatgttgggacacactccacacagaattcagtgagcaatggttctagtttaaattatttcaacatcaattatttcaaggatgcagcctcaagtggtgcttctagattagatttctcccaagatcctagtaaattcactgaccctgttaaagatgttttagaaaaaggtattccaacactccagtcaccgacagttgaggcttgtggttattcagacaggctaatacaaataacccgaggagattccactataacgtctcaggatattgcaaatgcagttgtcgcttatggtgtttggccatcatacctaactccagatgatgcgactgccattgacaaacctacacaacctgatacttcatccaatagattctacactctggatagtcgctcttggacatctgcctcatctggttggtggtggaaattgcctgatgcccttaagaacatgggtatctttggtgaaaacatgttttatcactttctagggagatctgggtacacaatacatgtacagtgtaattctagtaagttccatcagggtttgttgatagttgctgctatcccagagcatcaattggcatctgcgacaagtggaaatgtatcagttggatataaccacacccacccaggtgaacaaggtagagaagtggtgccatcacggacttctagtgataacaaaagacctagtgatgatagttggttgaactttgatgggacattacttggtaacctgcctatttatccccaccaatatattaatttaaggactaataattcagctacccttattttaccttatgttaatgctgtaccaatggattctatgcttagacacaataattggagtttagttataatcccaatatgccctcttcaagttcaaccaggagggacacaatccatacctataacagtatcaatcagccctatgttttcagaattttcagggccaaggaataaggttgtatttagtaccactcaa

SEQ ID NO: 27 - RNA, 1008 nt, Rhinovirus A
  source 1..1008: mol_type = genomic RNA
atgggtgcacaagtatctagacagaatgttgggacacactccacacagaattcagtgagcaatggttctagtttaaattatttcaacatcaattatttcaaggatgcagcctcaagtggtgcttctagattagatttctcccaagatcctagtaaattcactgaccctgttaaagatgttttagaaaaaggtattccaacactccagtcaccgacagttgaggcttgtggttattcagacaggctaatacaaataacccgaggagattccactataacgtctcaggatattgcaaatgcagttgtcgcttatggtgtttggccatcatacctaactccagatgatgcgactgccattgacaaacctacacaacctgatacttcatccaatagattctacactctggatagtcgctcttggacatctgcctcatctggttggtggtggaaattgcctgatgcccttaagaacatgggtatctttggtgaaaacatgttttatcactttctagggagatctgggtacacaatacatgtacagtgtaattctagtaagttccatcagggtttgttgatagttgctgctatcccagagcatcaattggcatctgcgacaagtggaaatgtatcagttggatataaccacacccacccaggtgaacaaggtagagaagtggtgccatcacggacttctagtgataacaaaagacctagtgatgatagttggttgaactttgatgggacattacttggtaacctgcctatttatccccaccaatatattaatttaaggactaataattcagctacccttattttaccttatgttaatgctgtaccaatggattctatgcttagacacaataattggagtttagttataatcccaatatgccctcttcaagttcaaccaggagggacacaatccatacctataacagtatcaatcagccctatgttttcagaattttcagggccaaggaataaggttgtatttagtaccactcaa